(19) United States
(12) Patent Application Publication
(10) Pub. No.: US 2023/0248815 A1
(43) Pub. Date: Aug. 10, 2023

(54) CHIMERIC RSV AND CORONAVIRUS PROTEINS, IMMUNOGENIC COMPOSITIONS, AND METHODS OF USE

(71) Applicant: Meissa Vaccines, Inc., Redwood City, CA (US)

(72) Inventors: Martin Moore, San Carlos, CA (US); Robert Jordan, Foster City, CA (US); Mariana Tioni, Pacifica, CA (US)

(21) Appl. No.: 18/010,436

(22) PCT Filed: Jun. 17, 2021
(86) PCT No.: PCT/US2021/037846

§ 371 Date: Dec. 14, 2022

Related U.S. Application Data
(60) Provisional application No. 63/194,092, filed on May 27, 2021.
(60) Provisional application No. 63/160,445, filed on Mar. 12, 2021.
(60) Provisional application No. 63/040,193, filed on Jun. 17, 2020.

(51) Int. Cl. A61K 39/12; C07K 14/135; C07K 14/165; C12N 15/62; C12N 15/86; A61P 31/14

(52) U.S. Cl. CPC A61K 39/12; C07K 14/135; C07K 14/165; C12N 15/62; C12N 15/86; A61P 31/14; A61K 2039/5254

(57) ABSTRACT

The invention relates generally to chimeric viral fusion proteins comprising the ectodomain and optionally the transmembrane domain of a first viral fusion protein (e.g., a spike protein of a coronavirus) and the cytoplasmic domain of a second viral fusion protein (e.g. RSV), immunogenic compositions comprising such chimeric proteins, and methods of use of same.

### CROSS REFERENCE TO RELATED APPLICATIONS

This application claims the benefit of and priority to U.S. Provisional Patent Application No. 63/040,193, filed Jun. 17, 2020; U.S. Provisional Patent Application No. 63/160,445, filed Mar. 12, 2021; and U.S. Provisional Patent Application No. 63/194,092, filed May 27, 2021, the disclosure of each of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Jun. 16, 2021, is named MSA-007WO_SL.txt and is 1,822,459 bytes in size.

### FIELD OF THE INVENTION

The invention relates generally to chimeric RSV and non-RSV proteins (e.g., non-pneumoviridae such as coronavirus), immunogenic compositions comprising such chimeric proteins, and methods of use of same.

### BACKGROUND

The Coronaviridae are a family of large, enveloped, single stranded RNA viruses responsible for respiratory and gastrointestinal disease in birds, fish, and mammals. The family derives its name from the hallmark appearance under electron microscopy of the crown-resembling spike proteins on virion surfaces, reported in the 1960s when the first coronavirus (CoV) strains were discovered (Kahn et al. (2005) The Pediatric Infectious Disease Journal, 24(11), S223-S227). Core features shared among CoVs include a virion diameter ranging 100-160 nm, the aforementioned spike (S) protein that binds to host cells, the membrane (M) glycoprotein, the envelope (E) protein, the nucleocapsid (N) protein, and a positive sense single stranded RNA genome ranging from 27-32 kb in length (Cui et al. (2019) Nature Reviews Microbiology, 17(3), 181-192). Based upon genetic phylogeny, all known human coronaviruses (HCoVs) are in the subfamily Orthocoronavirinae, specifically in the Alpha- and Betacoronavirus genera.

Certain HCoVs are globally endemic and cause seasonal upper or lower respiratory tract infections that are subclinical to moderate in severity in the immunocompetent host. These four strains (HCoV-229E, -NL63, -OC43, and -HKU1) collectively cause 10 to 30% of adult upper respiratory tract infections (Paules et al. (2020) JAMA, 323(8), 707-708) and have been detected in 8.2-10.8% of children with acute respiratory illnesses (Varghese et al. (2018) Journal of the Pediatric Infectious Diseases Society, 7(2), 151-158). In 2002 and 2012 two highly pathogenic beta-coronaviruses emerged from animal reservoirs to infect humans, triggering multinational epidemics of severe, life threatening respiratory disease. The pandemic caused by Severe Acute Respiratory Syndrome (SARS)-CoV resulted in over 8000 infected individuals in 29 countries who faced a 11% cumulative case fatality rate (CFR) (The World Health Organization (WHO) 2020), before it was contained eight months later. In contrast, Middle East Respiratory Syndrome (MERS)-CoV remains endemic in the Arabian Peninsula, where it has caused nearly 2500 cases and, with a 34% CFR, over 850 deaths in 27 countries (WHO 2019). No licensed therapeutic or preventive vaccine is available for SARS-CoV or MERS-CoV.

The COVID-19 global pandemic that began in Wuhan, China in December 2019 is caused by the highly transmissible Severe Acute Respiratory Syndrome Coronavirus 2 (SARS-CoV-2, Coronaviridae Study Group of the International Committee on Taxonomy of Viruses (2020) Nat Microbiol. 5(4):536-544). COVID-19 has an overall mortality rate of approximately 2% in the elderly and patients with serious underlying medical conditions such as heart or lung disease and diabetes. As of May 18, 2021, there were 163,312,429 confirmed cases of SARS-CoV-2 infection with a total of 3,386,825 deaths worldwide (WHO dashboard, at website covid19.who.int/).

SARS-CoV-2 is an enveloped RNA virus that relies on its surface glycoprotein, spike, for entry into host cells (Letko et al. (2020) Nat Microbiol. 5(4):562-569, Shang et al. (2020) Proc Natl Acad Sci USA. 117(21):11727-11734). The spike protein is a type I fusion protein and forms a trimer that protrudes on the viral membrane giving the virus its characteristic appearance of a crown under electron microscopy (Turofiovn el al. (2020) Science 370(6513):203-208; Li (2005) Annu Rev Virol. 3(1):237-261). The angiotensin converting enzyme 2 (ACE2) has been identified as a cellular receptor for SARS-CoV-2 spike (Letko et al., supra; Hoffmann et al. (2020) Cell 181(2):271-280). Disrupting the interaction of ACE2 and the receptor binding domain (RBD) of spike is at the core of vaccine design and therapeutics. Currently, three COVID-19 vaccines have been approved for emergency use in the U.S. (CDC, “Authorized and Recommended Vaccines” at website cdc.gov/coronavirus/2019-ncov/vaccines/different-vaccines.html). The three vaccines are based on SARS-CoV-2 spike protein and their high level of efficacy has validated spike as a protective antigen. However, despite existing vaccines, as of May 20, 2021, only 1.56 billion vaccine doses have been administered, equal to 20 doses for every 100 people. Some countries have not reported the administration of any vaccine doses.

All the EUA vaccines currently in use are delivered intramuscularly and none of them is live attenuated. Live attenuated vaccines (LAV) often use the same route of entry as the pathogen they target and replicate in the host mimicking natural infection without causing disease. As a result, LAV generate mucosal immunity at the site of infection, blocking the pathogen at the earliest phases of infection thus helping control systemic spread (Holngren et al. (2005) Nat. Med 11(4 Suppl):S45-53). In the case of influenza infection, it has been shown that LAV induce better mucosal IgA and cell-mediated immunity relative to other vaccine types, eliciting a longer lasting broader immune response that more closely resembles natural immunity (Cox et al. (2004) Scand J Immunol. 59(1):1-15). Furthermore, comparison of intramuscularly and intranasally administered vaccines against SARS-CoV in mice showed that serum IgA was only induced following intranasal vaccination (See et al. (2006) J Gen Virol. 87(Pt 3):641-650) and only intranasal vaccination provided protection in both upper and lower respiratory tract (Hassan et al. (2020) Cell 183(1):169-184.e13). For SARS-CoV-2 in particular, it has been reported that the early antibody response is dominated by IgA and that mucosal IgA are highly neutralizing (Sterlin et al. (2021) Sci Transl Med. 13(577):eabd2223), underscoring the importance of developing an intranasal vaccine capable of eliciting mucosal immunity. According to the WHO vaccine tracker, there are currently 101 vaccines in clinical trials, only 3 of which are intranasal live attenuated vaccines (“The Landscape of candidate vaccines in clinical development”, at website who.int/publications/m/item/draft-landscape-of-covid-19-candidate-vaccines as of May 14, 2021, prepared by WHO). These candidate live attenuated vaccines, however, may be susceptible to the high rate of RNA recombination observed in Coronaviruses that can result in loss of attenuation during co-infection with wild-type Coronaviruses.

Accordingly, there is a need in the art for immunogenic compositions, including vaccines, to prevent or lessen the severity of COVID-19 disease. The development of a needle-free, intranasal vaccine that is not prone to recombination and that generates both mucosal and humoral immune responses and that can be produced with high yields is needed.

### SUMMARY OF THE INVENTION

The disclosure is based, in part, on the discovery of a chimeric protein comprising portions of two viral fusion proteins which can be used in an immunogenic composition (e.g., a vaccine) for the prevention of a viral infection. The chimeric proteins described herein can be used in a vaccine construct that includes components of an RSV virus (e.g. proteins encoded by codon-deoptimized RSV genes) but that expresses the fusion protein on the surface of the virus. The chimeric protein, having a portion of a first fusion protein (e.g., an ectodomain of a fusion protein) and a portion of a second fusion protein (e.g., a cytoplasmic tail of a second fusion protein), promotes proper assembly of the chimeric protein into RSV particles.

In certain embodiments, the disclosure relates to a chimeric protein comprising a fusion protein from a non-RSV virus (any virus that is not RSV), such as a coronavirus spike protein or “S protein”; e.g., a SARS-CoV-2 spike protein, and an RSV F protein which can be used in an immunogenic composition (e.g., a vaccine) for the prevention of a coronavirus infection (e.g., a SARS-CoV-2 infection). The chimeric proteins described herein can be used in a vaccine construct that includes components of an RSV virus (e.g. codon-deoptimized RSV proteins) but that expresses the non-RSV fusion protein (e.g., the chimeric coronavirus S protein/RSV F protein) on the surface of the virus. The chimeric protein, having a portion of a non-RSV fusion protein (e.g., an ectodomain and optionally a transmembrane portion) and a portion of an RSV F protein (e.g., a cytoplasmic tail portion), promotes proper assembly of the chimeric protein into RSV particles.

In other embodiments, the cytoplasmic tail portion is a non-RSV fusion protein, such as the HA protein of Orthomyxoviridae (e.g., influenza virus); the Env protein of Retroviridae; the F and/or HN proteins of Paramyxoviridae (e.g., parainfluenza, measles, and mumps viruses); the S protein of Coronaviridae; the GP protein of Filoviridae; the GP and/or SSP proteins of Arenaviridae; the E1/E2 protein of Togaviridae; the E (e.g., in TBEV) or E1/E2 (e.g., in HCV) protein of Flaviviridae; the GN/GC protein of Bunyaviridiae; the G protein of Rhabdoviridae (VSV and rabies); the gB, gD, and/or gH/L protein of Herpesviridae; one or more of a complex of 8 proteins in poxviridae; and the S and/or L protein of Hepadnaviridae

In certain embodiments, the disclosure relates to an immunogenic composition comprising a chimeric protein as described herein, together with one or more RSV proteins (e.g., an NS1 and NS2 protein, wherein the NS1 and/or NS2 protein is optionally encoded by a codon-deoptimized gene). Although the immunogenic compositions described herein can, in certain embodiments, include a G gene, in other embodiments, the immunogenic composition (e.g., vaccine) does not include an RSV G gene. Without wishing to be bound by theory, it is believed that the RSV G gene is not needed, because certain fusion proteins, such as the coronavirus S protein, mediates both receptor attachment and virus-cell fusion. Indeed, the coronavirus spike protein is fully functional, necessary and sufficient for viral entry. A recombinant RSV-spike virus lacking G and F proteins can enter host cells, as described in Example 2 herein, indicating that the recombinant virus relies entirely on the chimeric coronavirus spike/RSV F protein for entry. Further, by removing RSV G and F, the resulting immunogenic composition should not be inhibited by pre-existing RSV immunity because known RSV neutralizing antibodies are primarily against F or against G.

In certain embodiments, the disclosure relates to a vaccine comprising a chimeric fusion protein as described herein that is administered intranasally, a needle-free route that is advantageous for global immunization. The intranasal route is similar to the natural route of infection of SARS-CoV-2 and generates both mucosal and humoral immune responses in AGMs without any adjuvant formulation, Modeling based on yields from the production of vaccines disclosed herein projected a potential dose output of hundreds of millions of doses per annum in a modestly sized facility using high intensity bioreactor systems. Mucosally delivered live attenuated vaccines such as those described herein entail minimal downstream processing and may be less expensive to produce than existing vaccines. In addition, needle-free delivery reduces supply risks. The vaccines described herein are suitable as a primary vaccine or as a heterologous booster.

Accordingly, in one aspect, the disclosure relates to a chimeric protein comprising an ectodomain of a SARS-CoV-2 spike protein and a cytoplasmic tail portion of an RSV fusion (F) protein. In certain embodiments, the chimeric protein comprises, in an N- to C-terminal direction, the ectodomain of the SARS-CoV-2 spike protein, and the cytoplasmic tail portion of the RSV fusion (F) protein. In certain embodiments, the chimeric protein further comprises a transmembrane domain of a SARS-CoV-2 spike protein. In certain embodiments, the chimeric protein further comprises a transmembrane domain of an RSV fusion protein.

In certain embodiments, the chimeric protein comprises a sequence selected from the group consisting of SEQ ID NOs: 1-6, 62, 68, 74, 80, 86, 92, 98, and 110 or a variant thereof having at least about 85% (e.g., at least about 90%, at least about 95%, at least about 96%, at least about 97%, at least about 98%, or at least about 99%) sequence identity to a sequence selected from the group consisting of SEQ ID NOs: 1-6, 62, 68, 74, 80, 86, 92, 98, and 110.

In another aspect, the disclosure relates to an immunogenic composition comprising live (e.g., live attenuated) chimeric virus comprising a nucleic acid encoding a chimeric protein comprising an ectodomain of a SARS-CoV-2 spike protein and a cytoplasmic tail portion of an RSV fusion (F) protein. In certain embodiments, the nucleic acid encodes a chimeric protein comprising, in an N- to C-terminal direction, the ectodomain of the SARS-CoV-2 spike protein and the cytoplasmic tail portion of the RSV fusion (F) protein. In certain embodiments, the nucleic acid comprises a chimeric protein further comprising a transmembrane domain of a SARS-CoV-2 spike protein. In certain embodiments, the nucleic acid comprises a chimeric protein further comprising a transmembrane domain of an RSV fusion protein.

In certain embodiments, the nucleic acid encodes a chimeric protein comprising a sequence selected from the group consisting of SEQ ID NOs: 1-6, 62, 68, 74, 80, 86, 92, 98, and 110 or a variant thereof having at least about 85% (e.g., at least about 90%, at least about 95%, at least about 96%, at least about 97%, at least about 98%, or at least about 99%) sequence identity to a sequence selected from the group consisting of SEQ ID NOs: 1-6, 62, 68, 74, 80, 86, 92, 98, and 110. In certain embodiments, the nucleic acid comprises a sequence selected from the group consisting of SEQ ID NOs: 7-12, 63, 69, 75, 81, 87, 93, 99, and 111, or a fragment or variant thereof having at least about 85% (e.g., at least about 90%, at least about 95%, at least about 96%, at least about 97%, at least about 98%, or at least about 99%) sequence identity to a sequence selected from the group consisting of SEQ ID NOs: 7-12, 63, 69, 75, 81, 87, 93, 99, and 111, or an RNA counterpart of any of the foregoing, or a complementary sequence of any of the foregoing.

It is understood that for viral nucleic acid sequences expressed as DNA sequences (i.e., using “T” nucleotides), the corresponding RNA sequence, in which “U” nucleotides are substituted for “T” nucleotides, is also contemplated. In addition, it is understood that where antigenomic sequences (e.g., as found in an expression vector) are provided, the complementary sequence, as would be found in an immunogenic composition (e.g., the genome of a virus and/or a vaccine sequence), is also contemplated.

In certain embodiments, the live chimeric virus further comprises an NS1 and/or an NS2 protein of RSV. In certain embodiments, the live chimeric virus does not comprise a gene that encodes RSV G protein.

In certain embodiments, the immunogenic composition further comprises an adjuvant and/or other pharmaceutically acceptable carrier. In certain embodiments, the adjuvant is an aluminum gel, aluminum salt, or monophosphoryl lipid A. In certain embodiments, the adjuvant is an oil-in-water emulsion optionally comprising α-tocopherol, squalene, and/or a surfactant.

In another aspect, the disclosure relates to a method for immunizing a subject against a SARS-CoV-2 virus, the method comprising administering to the subject an effective amount of an immunogenic composition as described herein. In certain embodiments, the administration is intranasal administration. In certain embodiments, the immunogenic composition is administered a dose of between about 10³ and about 10⁶. In certain embodiments, administration of the immunogenic composition induces a SARS-CoV-2 spike-specific mucosal IgA response or generates serum neutralizing antibodies.

In another aspect, the disclosure relates to a nucleic acid encoding a chimeric protein as described herein. In certain embodiments, the nucleic acid comprises a sequence selected from the group consisting of SEQ ID NOs: 7-12, 63, 69, 75, 81, 87, 93, 99, and 111 or a fragment of variant thereof having at least about 85% (e.g., at least about 90%, at least about 95%, at least about 96%, at least about 97%, at least about 98%, or at least about 99%) sequence identity to a sequence selected from the group consisting of SEQ ID NOs: 7-12, 63, 69, 75, 81, 87, 93, 99, and 111, or an RNA counterpart of any of the foregoing, or a complementary sequence of any of the foregoing.

In another aspect, the disclosure relates to a vector comprising a nucleic acid as described herein. In certain embodiments, the vector is selected from a plasmid or a bacterial artificial chromosome. In certain embodiments, the vector is a BAC comprising a sequence selected from the group consisting of SEQ ID NOs: 54-59, 66, 67, 72, 73, 78, 79, 84, 85, 90, 91, 96, 97, 102, 103, 114, 115, and 131-136 or a variant thereof having at least about 85% (e.g., at least about 90%, at least about 95%, at least about 96%, at least about 97%, at least about 98%, or at least about 99%) sequence identity to a sequence selected from the group consisting of SEQ ID NOs: 54-59, 66, 67, 72, 73, 78, 79, 84, 85, 90, 91, 96, 97, 102, 103, 114, 115, and 131-136.

In another aspect, the disclosure relates to an isolated recombinant particle comprising an NS1 and/or an NS2 protein of RSV and a chimeric SARS-CoV-2 spike protein-RSV fusion (F) protein as described herein. In certain embodiments, the isolated recombinant particle comprises a live attenuated chimeric RSV-SARS-CoV-2 genome or antigenome.

In another aspect, the disclosure relates to a live attenuated chimeric RSV-SARS-CoV-2 antigenome comprising a sequence selected from the group consisting of SEQ ID NOs: 13-18, 65, 71, 77, 83, 89, 95, 101, 104-109, and 113 or a variant thereof having at least about 85% (e.g., at least about 90%, at least about 95%, at least about 96%, at least about 97%, at least about 98%, or at least about 99%) sequence identity to a sequence selected from the group consisting of SEQ ID NOs: 13-18, 65, 71, 77, 83, 89, 95, 101, 104-109, and 113, or an RNA counterpart of any of the foregoing, or a complementary sequence of any of the foregoing.

These and other aspects and features of the invention are described in the following detailed description and claims.

### BRIEF DESCRIPTION OF THE DRAWINGS

In order to understand the invention and to demonstrate how it may be carried out in practice, embodiments are now described, by way of non-limiting example only, with reference to the accompanying drawings in which:

FIG. 1 is a schematic showing the design of MV-014-212. In MV-014-212, the NS1 and NS2 genes are deoptimized and the RSV SH, G and F genes are deleted and replaced by a gene encoding a chimeric protein spike-F. The amino acid sequence at the junction is shown below the block graphic. The transmembrane domain of spike is represented in light grey, to the left, and the cytoplasmic tail of F is depicted in dark grey, to the right. The reporter virus MVK-014-212, encoding the fluorescent protein mKate2 in the first gene position, is schematically shown at the bottom or the panel. NTD: N-terminal domain. RBD: Receptor binding domain. S1: subunit S1. S2: Subunit S2. S1/S2 and S2′: protease cleavage sites. FP: fusion peptide. IFP: Internal fusion peptide. HR1 and 2: heptad repeats 1 and 2. TM: transmembrane domain. CT: cytoplasmic tail. FIG. 1 discloses SEQ ID NO: 139.

FIG. 2 is a schematic depicting the sequences of the C-termini of the candidates, showing the different positions of the junction between the RSV F protein cytoplasmic tail and the SARS-CoV-2 spike protein transmembrane domain. The candidates were designed to contain an mKate2 gene as a fluorescent marker to follow rescue and propagation in culture. The 7 candidates listed in this figure (and in TABLE 3) were evaluated by their ability to rescue (defined as the generation of red fluorescent foci) and to grow to titers of 10⁵ PFU/mL or higher. MV-014-212 was chosen to pursue further investigation. The sequence of SARS-CoV-2 spike shows amino acids 1198-1273 of SEQ ID NO: 23. The sequence of MV 014-210 shows amino acids 1198-1278 of SEQ ID NO: 1. The sequence of MV 014-211 shows amino acids 1198-1278 of SEQ ID NO: 2. The sequence of MV 014-212 shows amino acids 1198-1290 of SEQ ID NO: 3. The sequence of MV 014-213 shows amino acids 1198-1284 of SEQ ID NO: 98. The sequence of MV 014-220 shows amino acids 1198-1275 of SEQ ID NO: 4. The sequence of MV 014-230 shows amino acids 1198-1268 of SEQ ID NO: 5. The sequence of MV 014-240 shows amino acids 1198-1271 of SEQ ID NO: 6. The sequence of MV 014-215 shows amino acids 1198-1260 of SEQ ID NO: 110. The sequence of RSV F (the C-terminal portion of the RSV F protein) is shown and provided at SEQ ID NO: 130.

FIG. 3 is a schematic showing the design of the BAC_DB1_mKate vector used to create a chimeric RSV/coronavirus vaccine.

FIGS. 4A-4C provides brightfield and fluorescence images of cell monolayers showing the propagation of MV-014-210 mediated by the fusion spike-F protein.

FIG. 5 is a schematic showing the rescue of recombinant MV-014-212 virus and viruses derived from it.

FIG. 6 provides micrographs showing syncytia formed by MV-014-212 and derived recombinant viruses. Micrographs were taken at a total amplification of 100× under phase contrast or using TRITC filter.

FIG. 7 shows a schematic of a coronavirus spike protein, its glycosylation sites (short black bars), and its furin cleavage site.

FIG. 8A is a Western blot showing full-length purified SARS-CoV-2 spike protein lacking the furin cleavage site (lane 1), MVK-014-212 (lane 2), MV-014-212 (lane 3), mock-infected Vero cell lysate (lane 4), blank, (water, lane 5). The molecular weight marks correspond to the migration of the BIO-RAD Precision Plus Protein Dual Color Standards (Cat #1610374). FIG. 8B is a graph showing multicycle replication kinetics of MV-014-212 compared with RSV A2 in serum-free Vero cells. Cells were infected at an MOI of 0.01 and incubated at 32° C. Cells and supernatants were collected at 0, 12, 24, 48, 72, 96, and 120 hours post-infection. Titers of the samples were determined by plaque assay in Vero cells. Data points represent the means of two replicate wells and error bars represent the standard deviation. FIG. 8C is a graph showing multicycle replication kinetics of MV-014-212 compared with MVK-014-212 in serum-free Vero cells. Cells were infected at an MOI of 0.01 and incubated at 32° C. Cells and supernatants were collected at 0, 3, 24, and 72 hours post-infection. Titers of the samples were determined by plaque assay in Vero cells. Data points represent the means of three replicate wells and error bars represent the standard deviation. FIG. 8D is a graph showing the results of a short-term thermal stability assay. Virus stocks of MV-014-212 prepared in Williams E+SPG or prepared in SPG alone were incubated for 6 h at −80° C., 4° C., −20° C. and room temperature and the titer after incubation was determined by plaque assay.

FIG. 9 shows a schematic of the genetic stability experiment described in Example 3. Briefly, the genetic stability of MV-014-212 was examined by serial passaging in vitro. Three flasks of subconfluent Vero cells were infected with an aliquot of MV-014-212 and passaged the 3 lineages in parallel for 10 passages. Using RT-PCR followed by Sanger sequencing, the sequence of the entire genome of the starting stock (passage 0) and passage 10 for all lineages was determined. The accumulation of variants was not detected, suggesting that the MV-014-212 vaccine candidate is stable.

FIG. 10 is a schematic overview of a SARS-CoV-2 challenge test performed on African Green Monkeys (AGM) inoculated with MV-014-212, wt RSV, or PBS. Nasal swabs (NS) were obtained on Days 1 through 12. Bronchoalveolar lavage (BAL) were collected on Days 2, 4, 6, 8, 10 and 12. Viral shedding in NS and BAL samples were determined by plaque assay using fresh samples. On Day 28 post-inoculation AGMs were challenged with wt SARS-CoV-2. Nasal swabs were obtained every day from Day 29 through 38. Bronchoalveolar lavage was obtained on alternate days starting on Day 30 to Day 38. RT-qPCR was used for detecting SARS-CoV-2 shedding in NS and BAL samples.

FIGS. 11A-B provides graphs showing attenuation of MV-014-212 in the upper and lower respiratory tract of African Green Monkeys (AGM). Viral titer in nasal swabs (FIG. 11A) or bronchoalveolar lavage (BAL) (FIG. 11B) from AGMs following inoculation with MV-014-212 or wt RSV A2 were measured by plaque assay on Vero cells. On Days 1 through 12 post-inoculation nasal swabs were collected in Williams E supplemented with SPG. Viral titer in BAL were measured on Days 2, 4, 6, 8, 10, and 12 post-inoculation. The box in the graph defines the 25th and 75th percentile with error bars showing the maximum and minimum values. The horizontal line in the box is the mean value of the data points for each time point. The dotted line represents the LOD (50 PFU/mL).

FIGS. 12A-D show graphs of the results of two independent experiments performed in cotton rats. In experiment 1, cotton rats (n=5 per group) were inoculated with 1×10⁵ PFU of biologically derived TN-12, Memphis 37 (M37) or recombinant A2 (rA2) RSV strains. On Day 3, 5 and 7 cotton rat nasal and lung tissues were homogenized in HBSS+10% SPG for titer determination by plaque assay. In this experiment only Day 5 nasal and lung tissues were collected for the rA2 group. In experiment 2, cotton rats (n=6) were inoculated with 5×10⁵ PFU of rA2. On Day 2, 5 and 7 cotton rat nasal and lung tissues were homogenized in HBSS+10% SPG for titer determination by plaque assay. Plaque assay was performed in HEp-2 cells using clarified nasal and lung homogenates diluted in EMEM. Plaques were visualized by immunostaining with RSV polyclonal antibodies in experiment 1 and by crystal violet staining in experiment 2. FIG. 12A shows replication kinetics of TN-12, M37 and rA2 in the nose. FIG. 12B compares nasal titers of TN-12, M37 and rA2 on day 5. FIG. 12C shows replication kinetics of biological TN-12 and Memphis 37 and rA2 in the lungs. FIG. 12D compares lung titers on day 5 of TN-12, M37 and rA2. The result showed that the rA2 nose titers were comparable to biologically derived TN12 and M37 but rA2 lung titers were approximately 2 log lower compared to biologically derived RSV strains.

FIG. 13 provides graphs demonstrating the protection of MV-014-212 vaccinated AGMs against wt SARS-CoV-2 challenge. wt SARS-CoV-2 sgRNA in nasal swab samples from AGMs inoculated with MV-014-212, wt RSV A2 or PBS (Mock) following challenge. At Day 28, animals were challenged with 1.0×10⁶ TCID₅₀ of wt SARS-CoV-2 by intranasal and intratracheal inoculation. Nasal swabs collected on Days 1, 2, 4, and 6 post-challenge with wt SARS-CoV-2 were shown. The level of SARS-CoV-2 sgRNA was determined by RT-qPCR. The dashed line represents the LOD of 50 genome equivalents (GE)/mL. (sgRNA=sub-genomic RNA.)

FIG. 14A is a schematic showing a sandwich assay used to measure spike-specific nasal IgA. The SARS-CoV-2 spike protein is used as the antigen (2) that binds IgA from a sample (e.g., nasal swab sample). An IgA standard curve was generated by replacing the spike protein with an IgA capture Ab (FIG. 14B), with “OD at 450 nm” on the Y-axis and “[IgA] (pg/mL)” on the x-axis.

FIG. 15A provides a graph showing spike specific serum IgG in MV-014-212-inoculated AGMs. Antibodies specific to SARS-CoV-2 spike protein were measured by ELISA using serum collected on Day 25 from AGM inoculated with MV-014-212, wt RSV A2, or PBS (mock). The titer is expressed as ELISA units (ELU)/mL that were calculated by comparison to a standard curve generated from pooled human convalescent serum. FIG. 15B provides a graph showing measurement of IgA antibodies specific to SARS-CoV-2 spike protein by ELISA using nasal swabs collected on Day 25 post inoculation. The Log 2 of the ratio of the values obtained at day 25 over day 1 are shown. The calculated ELU/mL concentration was obtained from standard curve generated from total purified human IgA using a capture ELISA. FIG. 15C provides graphs showing neutralization titres (NT50) with sera from two AGM immunized with MV-014-212, before (“pre”) and 25 days after immunization (“imm”) with MV-014-212. WHO std is a human convalescent serum cocktail control at 100 IU/mL. NT50 were obtained from fitting the inhibition curves to the option “[Inhibitor] vs. normalized response—Variable slope” in GraphPad Prism. WHO Std. is a pool of convalescent sera at 100 IU/mL. The % Inhibition was calculated as described in Example 3 and the inhibition curves are shown in FIG. 16. The curves corresponding to samples AGM #1 Pre (all viruses) and AGM #2 (RSV) did not show significant inhibition and could not be fitted. LOD is 5, indicated with a horizontal dashed line. The data represents the average of two replicates and the error bars correspond to S.D. The table on the right shows the average NT50 for each reporter virus.

FIG. 16 provides graphs showing neutralizing curves. The percentage of inhibition was calculated as described in Methods. The inhibition curves shown below were fitted using non-linear regression with the option “[Inhibitor] vs. normalized response—Variable slope” in GraphPad Prism. The measures correspond to the average of 2 replicates and the error bars are SD. The reporter virus corresponding to each assay is shown on top. The WHO STD is a pool of convalescent sera at 100 IU/mL.

FIG. 17 provides graphs showing neutralization assays with sera from AGMs #1 and #2, before (“pre”) and 25 days after immunization (“imm”) with MV-014-212. WHO std is a human convalescent serum cocktail control at 100 IU/mL. The graphs show the inhibition achieved with the highest concentration of serum or control (1:5). The percentage of inhibition was calculated as described in Methods. The data represents the average of two replicates and the error bars correspond to S.D.

FIG. 18 is a schematic of the neutralization assay as described in Example 3.

FIGS. 19A-D provide graphs showing that MV-014-212 elicited a Th1-biased immune response in ACE-2 mice. FIG. 19A shows ELISpot results for IFNg (left) or IL-5 (right) producing cells in ACE-2 mice. Splenocytes isolated from hACE-2 expressing mice (n=5) were collected on Day 28 post-inoculation and stimulated with a peptide pool that spanned the SARS-CoV-2 spike protein (pool), media, or the mitogen concanavalin A (Con A). IL-5 or IFNγ expressing T-cells were quantified by ELISpot assay. hACE-2 expressing mice were inoculated at Day 0 via the intranasal route with MV-014-212 or PBS. Control mice were vaccinated with purified SARS-CoV-2 spike protein adjuvanted with alum by intramuscular injection at Day −20 and Day 0. FIG. 19B provides a graph showing the log of the ratio of IFNγ to IL-5 expressing cells (as shown in FIG. 19A). FIG. 19C provides a graph showing the results of IgG1 and IgG2a ELISAs. Levels of IgG2a (left panel) and IgG1 (right panel) corresponding to Day 28 serum from hACE-2 mice vaccinated intranasally with PBS or MV-014-212 or intramuscularly with spike-alum, as determined by ELISA. The concentration of each immunoglobulin isotype was determined from standard curves generated with purified SARS-CoV-2 spike specific monoclonal IgG2a or IgG1 antibodies. FIG. 19D provides a graph showing the log of the ratio of IgG2a/IgG1 (as shown in FIG. 19C). Statistical analysis is a paired t-test. ***P<0.0005

### DETAILED DESCRIPTION

The disclosure is based, in part, on the discovery of a chimeric protein comprising portions of two viral fusion proteins which can be used in an immunogenic composition (e.g., a vaccine) for the prevention of a viral infection. The chimeric proteins described herein can be used in a vaccine construct that includes components of an RSV virus (e.g. codon-deoptimized RSV proteins) but that expresses the chimeric fusion protein on the surface of the virus. The chimeric protein, having a portion of a first fusion protein (e.g., an ectodomain of a fusion protein) and a portion of a second fusion protein (e.g., a cytoplasmic tail of a second fusion protein), promotes proper assembly of the chimeric protein into RSV particles.

In certain embodiments, the disclosure relates to a chimeric protein comprising a non-RSV fusion protein (e.g., a coronavirus spike protein or “S protein”; e.g., a SARS-CoV-2 spike protein) and an RSV F protein which can be used in an immunogenic composition (e.g., a vaccine) for the prevention of a viral infection (e.g., a SARS-CoV-2 infection). The chimeric proteins described herein can be used in a vaccine construct that includes components of an RSV virus (e.g. codon-deoptimized RSV proteins) but that expresses the fusion protein (e.g., the S protein) on the surface of the virus. The chimeric protein, having a portion of a non-RSV fusion protein (e.g., coronavirus S protein) and a portion of an RSV F protein, promotes proper assembly of the chimeric protein into RSV particles.

The disclosure further relates to nucleic acids encoding a chimeric protein comprising a portion (e.g., an ectodomain) of a first fusion protein and a portion (e.g., a cytoplasmic tail) of a second fusion protein and immunogenic compositions (e.g., vaccines) comprising the same. In certain embodiments, the immunogenic composition comprises RSV genes in addition to or other than the F gene, which may be codon deoptimized. Although the immunogenic compositions described herein can, in certain embodiments, include a G gene, in other embodiments, the immunogenic composition (e.g., vaccine) does not include an RSV G gene. Without wishing to be bound by theory, it is believed that the RSV G gene is not needed, because certain fusion proteins, such as the coronavirus S protein, mediates both receptor attachment and virus-cell fusion. Indeed, the coronavirus spike protein is fully functional, necessary and sufficient for viral entry. A recombinant RSV-spike virus lacking G and F proteins can enter host cells, as described in Example 2 herein, indicating that the recombinant virus relies entirely on the chimeric coronavirus spike/RSV F protein for entry. Further, by removing RSV G and F, the resulting immunogenic composition should not be inhibited by pre-existing RSV immunity because known RSV neutralizing antibodies are primarily against F or against G.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

Prior to describing the various embodiments, the following definitions are provided and should be used unless otherwise indicated.

The terms “protein” and “polypeptide” refer to compounds comprising amino acids joined via peptide bonds and are used interchangeably.

The term “portion” when used in reference to a protein (as in “a portion of a given protein”) refers to fragments of that protein. The fragments may range in size from four amino acid residues to the entire amino sequence lacking one amino acid.

The terms “chimeric respiratory syncytial virus (RSV)” or “chimeric coronavirus/RSV” refer to a nucleic acid that contains sufficient RSV genes to allow the genome or antigenome to replicate in host cells (e.g. Vero cells) and the sequence nucleic acid is altered to include at least one nucleic acid segment that contains a non-RSV (e.g., coronavirus) gene sequence or fragment. A chimeric RSV can include a non-RSV (e.g., coronavirus) and/or RSV gene wherein the codons are altered to be different from those naturally occurring even though the gene produces a polypeptide with an identical amino acid sequence to those naturally expressed. Different strains of chimeric RSV will have different nucleotide sequences and express proteins with different amino acid sequences that have similar functions. Thus, a chimeric RSV includes an non-RSV (e.g., coronavirus) gene and/or RSV gene wherein one or more genes from one strain are replaced from genes in alternative or second strain such that the nucleic acid sequence of the entire non-RSV or RSV genome is not identical to a non-RSV (e.g., coronavirus) or RSV found in nature. In certain embodiments, the chimeric RSV includes those strains where nucleic acids are deleted after a codon for starting translation in order to truncate the proteins expression, provided such truncation pattern for the genome is not found in naturally occurring virus. In certain embodiments, the chimeric RSV includes those that are infectious and can replicate in a human subject. As used herein the term “non-RSV” refers to any virus that is not RSV. In certain embodiments, the non-RSV virus is a virus that is not in the pneumoviridae family (i.e., is a non-pneumovirus). Any instance of the term “non-RSV” present herein can, in certain embodiments, be substituted with the term “a virus outside of the pneumoviridae family” or “a virus that is not in the pneumoviridae family.”

The term “chimera” or “chimeric” when used in reference to a polypeptide refers to the expression product of two or more coding sequences obtained from different sources such that they do not exist together in a natural environment, that have been cloned together and that, after translation, act as a single polypeptide sequence. The coding sequences include those obtained from the same or from different species of organisms. The present disclosure relates to chimeric RSV proteins, e.g., non-RSV (e.g., coronavirus)/RSV proteins. In certain embodiments, the chimeric RSV protein comprises a non-RSV fusion protein or portion or variant thereof and an RSV F protein or portion (e.g. cytoplasmic tail portion) or variant thereof.

The term “fusion protein” refers to a viral protein that mediates fusion of a viral membrane and a cell membrane, allowing the virus to enter and infect a cell. Fusion proteins contemplated for use in the chimeric proteins herein include at least a portion of the HA protein of Orthomyxoviridae (e.g., influenza virus); the Env protein of Retroviridae; the F and/or HN proteins of Paramyxoviridae (e.g., parainfluenza, measles, and mumps viruses); the S protein of Coronaviridae; the GP protein of Filoviridae; the GP and/or SSP proteins of Arenaviridae; the E1/E2 protein of Togaviridae; the E (e.g., in TBEV) or E1/E2 (e.g., in HCV) protein of Flaviviridae; the GN/GC protein of Bunyaviridiae; the G protein of Rhabdoviridae (VSV and rabies); the gB, gD, and/or gH/L protein of Herpesviridae; one or more of a complex of 8 proteins in poxviridae; and the S and/or L protein of Hepadnaviridae.

The term “coronavirus” refers to a group of RNA viruses that cause diseases (e.g., in mammals and birds. Coronaviruses cause seasonal upper or lower respiratory tract infections that are subclinical to moderate in severity in the immunocompetent host. Human coronaviruses include HCoV-229E, -NL63, -OC43, -HKU1, Severe Acute Respiratory Syndrome (SARS)-CoV, Middle East Respiratory Syndrome (MERS)-CoV, and SARS-CoV-2. Where the term coronavirus is used herein, SARS-CoV-2 is contemplated as a specific embodiment.

The term “homolog” or “homologous” when used in reference to a polypeptide refers to a high degree of sequence identity between two polypeptides, or to a high degree of similarity between the three-dimensional structures or to a high degree of similarity between the active site and the mechanism of action. In a preferred embodiment, a homolog has a greater than 60% sequence identity, and more preferably greater than 75% sequence identity, and still more preferably greater than 90% sequence identity, with a reference sequence.

As applied to polypeptides or polynucleotides, the term “substantial identity” means that two peptide or nucleotide sequences, when optimally aligned, such as by the programs “GAP” (Genetics Computer Group, Madison, Wis.), “ALIGN” (DNAStar, Madison, Wis.), Jotun Hein (Hein (2001) Proc. Pacific Symp. Biocomput. 179-190), using default gap weights, share at least 80 percent sequence identity, preferably at least 90 percent sequence identity, more preferably at least 95 percent sequence identity, e.g., at least 96 percent identity, at least 97 percent identity, at least 98 percent identity, at least 99 percent identity, at least 99.5 percent identity, at least 99.9 percent identity. Preferably, for polypeptides, residue positions which are not identical differ by conservative amino acid substitutions.

The terms “variant” and “mutant” when used in reference to a polypeptide (or the polynucleotide encoding such a polypeptide) refer to an amino acid sequence (or encoded amino acid sequence) that differs by one or more amino acids from another, usually related polypeptide. The variant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (in other words, additions), or both. Guidance in determining which and how many amino acid residues may be substituted, inserted or deleted without abolishing biological activity may be found using computer programs well known in the art, for example, DNAStar software. Variants can be tested in functional assays. Preferred variants have less than 10%, and preferably less than 5%, and still more preferably less than 2% changes (whether substitutions, deletions, and so on).

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (mRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “heterologous gene” refers to a gene encoding a factor that is not in its natural environment (i.e., has been altered by the hand of man). For example, a heterologous gene includes a gene from one species introduced into another species. A heterologous gene also includes a gene native to an organism that has been altered in some way (e.g., mutated, added in multiple copies, linked to a non-native promoter or enhancer sequence, etc.). Heterologous genes are distinguished from endogenous genes in that the heterologous gene sequences are typically joined to nucleotide sequences comprising regulatory elements such as promoters that are not found naturally associated with the gene for the protein encoded by the heterologous gene or with gene sequences in the chromosome, or are associated with portions of the chromosome not found in nature (e.g., genes expressed in loci where the gene is not normally expressed).

The term “polynucleotide” refers to a molecule comprised of two or more deoxyribonucleotides or ribonucleotides, preferably more than three, and usually more than ten. The exact size will depend on many factors, which in turn depends on the ultimate function or use of the oligonucleotide. The polynucleotide may be generated in any manner, including chemical synthesis, DNA replication, reverse transcription, or a combination thereof. The term “oligonucleotide” generally refers to a short length of single-stranded polynucleotide chain although it may also be used interchangeably with the term “polynucleotide.”

The term “nucleic acid” refers to a polymer of nucleotides, or a polynucleotide, as described above. The term is used to designate a single molecule, or a collection of molecules. Nucleic acids may be single stranded or double stranded, and may include coding regions and regions of various control elements, as described below.

The term “nucleic acid encoding a gene” or “a nucleic acid encoding” a specified polypeptide refers to a nucleic acid sequence comprising the coding region of a gene or in other words the nucleic acid sequence which encodes a gene product. The coding region may be present in either a cDNA, genomic DNA or RNA form. When present in a DNA form, the oligonucleotide, polynucleotide, or nucleic acid may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present disclosure may contain endogenous enhancers/promoters, splice junctions, intervening sequences, polyadenylation signals, etc. or a combination of both endogenous and exogenous control elements.

The term “recombinant” when made in reference to a nucleic acid molecule refers to a nucleic acid molecule which is comprised of segments of nucleic acid joined together by means of molecular biological techniques. The term “recombinant” when made in reference to a protein or a polypeptide refers to a protein molecule which is expressed using a recombinant nucleic acid molecule.

The terms “complementary” and “complementarity” refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

The term “homology” when used in relation to nucleic acids refers to a degree of complementarity. There may be partial homology or complete homology (i.e., identity). “Sequence identity” refers to a measure of relatedness between two or more nucleic acids or proteins, and is given as a percentage with reference to the total comparison length. The identity calculation takes into account those nucleotide or amino acid residues that are identical and in the same relative positions in their respective larger sequences. Calculations of identity may be performed by algorithms contained within computer programs such as “GAP” (Genetics Computer Group, Madison, Wis.) and “ALIGN” (DNAStar, Madison, Wis.). A partially complementary sequence is one that at least partially inhibits (or competes with) a completely complementary sequence from hybridizing to a target nucleic acid is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a sequence which is completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

The following terms are used to describe the sequence relationships between two or more polynucleotides: “reference sequence”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (Smith and Waterman, Adv. Appl. Math. 2: 482 (1981)) by the homology alignment algorithm of Needleman and Wunsch (Needleman and Wunsch, J. Mol. Biol. 48:443 (1970)), by the search for similarity method of Pearson and Lipman (Pearson and Lipman, Proc. Natl. Acad. Sci. (U.S.) 85:2444 (1988)), by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison.

In certain embodiments, term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

In certain embodiments, sequence “identity” refers to the number of exactly matching amino acids (expressed as a percentage) in a sequence alignment between two sequences of the alignment calculated using the number of identical positions divided by the greater of the shortest sequence or the number of equivalent positions excluding overhangs wherein internal gaps are counted as an equivalent position. For example, the polypeptides GGGGGG (SEQ ID NO: 19) and GGGGT (SEQ ID NO: 20) have a sequence identity of 4 out of 5 or 80%. For example, the polypeptides GGGPPP (SEQ ID NO: 21) and GGGAPPP (SEQ ID NO: 22) have a sequence identity of 6 out of 7 or 85%. In certain embodiments, any recitation of sequence identity expressed herein may be substituted for sequence similarity. Percent “similarity” is used to quantify the similarity between two sequences of the alignment. This method is identical to determining the identity except that certain amino acids do not have to be identical to have a match. Amino acids are classified as matches if they are among a group with similar properties according to the following amino acid groups: Aromatic—F Y W; hydrophobic—A V I L; Charged positive: R K H; Charged negative—D E; Polar—S T N Q.

The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present disclosure.

When used in reference to a double-stranded nucleic acid sequence such as a cDNA or genomic clone, the term “substantially homologous” refers to any probe that can hybridize to either or both strands of the double-stranded nucleic acid sequence under conditions of low to high stringency as described above.

When used in reference to a single-stranded nucleic acid sequence, the term “substantially homologous” refers to any probe that can hybridize (i.e., it is the complement of) the single-stranded nucleic acid sequence under conditions of low to high stringency as described above.

The terms “in operable combination”, “in operable order” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc.

Transcriptional control signals in eukaryotes comprise “promoter” and “enhancer” elements. Promoters and enhancers consist of short arrays of DNA sequences that interact specifically with cellular proteins involved in transcription (Maniatis, et al., Science 236:1237, 1987). Promoter and enhancer elements have been isolated from a variety of eukaryotic sources including genes in yeast, insect, mammalian and plant cells. Promoter and enhancer elements have also been isolated from viruses and are found in prokaryotes. The selection of a particular promoter and enhancer depends on the cell type used to express the protein of interest. Some eukaryotic promoters and enhancers have a broad host range while others are functional in a limited subset of cell types (for review, see Voss, et al., Trends Biochem. Sci., 11:287, 1986; and Maniatis, et al., supra 1987).

The terms “promoter element,” “promoter,” or “promoter sequence” as used herein, refer to a DNA sequence that, e.g., functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

Promoters may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue (e.g., leaves). Tissue specificity of a promoter may be evaluated by, for example, operably linking a reporter gene to the promoter sequence to generate a reporter construct, introducing the reporter construct into the genome of an organism such that the reporter construct is integrated into every tissue of the resulting transgenic organism, and detecting the expression of the reporter gene (e.g., detecting mRNA, protein, or the activity of a protein encoded by the reporter gene) in different tissues of the transgenic organism. The detection of a greater level of expression of the reporter gene in one or more tissues relative to the level of expression of the reporter gene in other tissues shows that the promoter is specific for the tissues in which greater levels of expression are detected. The term “cell type specific” as applied to a promoter refers to a promoter which is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining. Briefly, tissue sections are embedded in paraffin, and paraffin sections are reacted with a primary antibody which is specific for the polypeptide product encoded by the nucleotide sequence of interest whose expression is controlled by the promoter. A labeled (e.g., peroxidase conjugated) secondary antibody which is specific for the primary antibody is allowed to bind to the sectioned tissue and specific binding detected (e.g., with avidin/biotin) by microscopy.

Promoters may be constitutive or regulatable. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue.

In contrast, a “regulatable” or “inducible” promoter is one which is capable of directing a level of transcription of an operably linked nucleic acid sequence in the presence of a stimulus (e.g., heat shock, chemicals, light, etc.) which is different from the level of transcription of the operably linked nucleic acid sequence in the absence of the stimulus.

The enhancer and/or promoter may be “endogenous” or “exogenous” or “heterologous.” An “endogenous” enhancer or promoter is one that is naturally linked with a given gene in the genome. An “exogenous” or “heterologous” enhancer or promoter is one that is placed in juxtaposition to a gene by means of genetic manipulation (i.e., molecular biological techniques) such that transcription of the gene is directed by the linked enhancer or promoter. For example, an endogenous promoter in operable combination with a first gene can be isolated, removed, and placed in operable combination with a second gene, thereby making it a “heterologous promoter” in operable combination with the second gene. A variety of such combinations are contemplated (e.g., the first and second genes can be from the same species, or from different species).

Efficient expression of recombinant DNA sequences in eukaryotic cells can require expression of signals directing the efficient termination and polyadenylation of the resulting transcript. Transcription termination signals are generally found downstream of the polyadenylation signal and are a few hundred nucleotides in length. The term “poly(A) site” or “poly(A) sequence” as used herein denotes a DNA sequence which directs both the termination and polyadenylation of the nascent RNA transcript. Efficient polyadenylation of the recombinant transcript is desirable, as transcripts lacking a poly(A) tail are unstable and are rapidly degraded. The poly(A) signal utilized in an expression vector may be “heterologous” or “endogenous.” An endogenous poly(A) signal is found naturally at the 3′ end of the coding region of a given gene in the genome. A heterologous poly(A) signal is one which has been isolated from one gene and positioned 3′ to another gene. A commonly used heterologous poly(A) signal is the SV40 poly(A) signal. The SV40 poly(A) signal is contained on a 237 bp BamHI/BclI restriction fragment and directs both termination and polyadenylation.

The term “vector” refers to nucleic acid molecules that transfer DNA segment(s) from one cell to another. The term “vehicle” is sometimes used interchangeably with “vector.”

The terms “expression vector” or “expression cassette” refer to a recombinant nucleic acid containing a desired coding sequence and appropriate nucleic acid sequences used for the expression of the operably linked coding sequence in a particular host organism. Nucleic acid sequences used for expression in prokaryotes typically include a promoter, an operator (optional), and a ribosome binding site, often along with other sequences. Eukaryotic cells are known to utilize promoters, enhancers, and termination and polyadenylation signals.

The term “host cell” refers to any cell capable of replicating and/or transcribing and/or translating a heterologous gene. Thus, a “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

A “selectable marker” is a nucleic acid introduced into a recombinant vector that encodes a polypeptide that confers a trait suitable for artificial selection or identification (see also, “reporter gene” below), e.g., beta-lactamase confers antibiotic resistance, which allows an organism expressing beta-lactamase to survive in the presence antibiotic in a growth medium. Another example is thymidine kinase, which makes the host sensitive to ganciclovir selection. It may be a screenable marker that allows one to distinguish between wanted and unwanted cells based on the presence or absence of an expected color. For example, the lac-z-gene produces a beta-galactosidase enzyme which confers a blue color in the presence of X-gal (5-bromo-4-chloro-3-indolyl-β-D-galactoside). If recombinant insertion inactivates the lac-z-gene, then the resulting colonies are colorless. There may be one or more selectable markers, e.g., an enzyme that can complement to the inability of an expression organism to synthesize a particular compound required for its growth (auxotrophic) and one able to convert a compound to another that is toxic for growth. URA3, an orotidine-5′ phosphate decarboxylase, is necessary for uracil biosynthesis and can complement ura3 mutants that are auxotrophic for uracil. URA3 also converts 5-fluoroorotic acid into the toxic compound 5-fluorouracil. Additional contemplated selectable markers include any genes that impart antibacterial resistance or express a fluorescent protein. Examples include, but are not limited to, the following genes: ampr, camr, tetr, blasticidinr, neor, hygr, abxr, neomycin phosphotransferase type II gene (nptII), p-glucuronidase (gus), green fluorescent protein (gfp), egfp, yfp, mCherry, p-galactosidase (lacZ), lacZa, lacZAM15, chloramphenicol acetyltransferase (cat), alkaline phosphatase (phoA), bacterial luciferase (luxAB), bialaphos resistance gene (bar), phosphomannose isomerase (pmi), xylose isomerase (xylA), arabitol dehydrogenase (atlD), UDP-glucose:galactose-1-phosphate uridyltransferaseI (galT), feedback-insensitive a subunit of anthranilate synthase (OASA1D), 2-deoxyglucose (2-DOGR), benzyladenine-N-3-glucuronide, E. coli threonine deaminase, glutamate 1-semialdehyde aminotransferase (GSA-AT), D-amino acidoxidase (DAAO), salt-tolerance gene (rstB), ferredoxin-like protein (pflp), trehalose-6-P synthase gene (AtTPS1), lysine racemase (lyr), dihydrodipicolinate synthase (dapA), tryptophan synthase beta 1 (AtTSB1), dehalogenase (dhlA), mannose-6-phosphate reductase gene (M6PR), hygromycin phosphotransferase (HPT), and D-serine ammonialyase (dsdA).

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “label receptor” refers to incorporation of a heterologous polypeptide in the receptor. A label includes the incorporation of a radiolabeled amino acid or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as 35S or 131I) fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance.

An “immunogenic composition” refers to one or more nucleic acids or proteins capable of giving rise to an immune response in a subject. An immunogenic composition can include, for example, a virus or portion thereof (e.g., a live or dead virus, viral particle, or virus-like particle (VLP)) which, in certain embodiments, can be administered as a vaccine.

In certain embodiments, the disclosure relates to recombinant polypeptides comprising sequences disclosed herein or variants or fusions thereof wherein the amino terminal end or the carbon terminal end of the amino acid sequence are optionally attached to a heterologous amino acid sequence, label, or reporter molecule.

In certain embodiments, the disclosure relates to the recombinant vectors comprising a nucleic acid encoding a polypeptide disclosed herein or fusion protein thereof.

In certain embodiments, the recombinant vector optionally comprises a mammalian, human, insect, viral, bacterial, bacterial plasmid, yeast associated origin of replication or gene such as a gene or retroviral gene or lentiviral LTR, TAR, RRE, PE, SLIP, CRS, and INS nucleotide segment or gene selected from tat, rev, nef, vif, vpr, vpu, and vpx or structural genes selected from gag, pol, and env.

In certain embodiments, the recombinant vector optionally comprises a gene vector element (nucleic acid) such as a selectable marker region, lac operon, a CMV promoter, a hybrid chicken B-actin/CMV enhancer (CAG) promoter, tac promoter, T7 RNA polymerase promoter, SP6 RNA polymerase promoter, SV40 promoter, internal ribosome entry site (IRES) sequence, cis-acting woodchuck post regulatory element (WPRE), scaffold-attachment region (SAR), inverted terminal repeats (ITR), FLAG tag coding region, c-myc tag coding region, metal affinity tag coding region, streptavidin binding peptide tag coding region, polyHis tag coding region, HA tag coding region, MBP tag coding region, GST tag coding region, polyadenylation coding region, SV40 polyadenylation signal, SV40 origin of replication, Col E1 origin of replication, f1 origin, pBR322 origin, or pUC origin, TEV protease recognition site, loxP site, Cre recombinase coding region, or a multiple cloning site such as having 5, 6, or 7 or more restriction sites within a continuous segment of less than 50 or 60 nucleotides or having 3 or 4 or more restriction sites with a continuous segment of less than 20 or 30 nucleotides.

The term “reporter gene” refers to a gene encoding a protein that may be assayed. Examples of reporter genes include, but are not limited to, modified katushka, mkate and mkate2 (See, e.g., Merzlyak et al., (2007) Nat. Methods 4, 555-557 and Shcherbo et al. (2008) Biochem. J. 418, 567-574), luciferase (See, e.g., deWet et al., (1987) Mol. Cell. Biol. 7:725 and U.S. Pat. Nos. 6,074,859; 5,976,796; 5,674,713; and 5,618,682; all of which are incorporated herein by reference), green fluorescent protein (e.g., GenBank Accession Number U43284; a number of GFP variants are commercially available from ClonTech Laboratories, Palo Alto, Calif.), chloramphenicol acetyltransferase, beta-galactosidase, alkaline phosphatase, and horse radish peroxidase.

The term “wild-type” when made in reference to a gene refers to a gene which has the characteristics of a gene isolated from a naturally occurring source. The term “wild-type” when made in reference to a gene product refers to a gene product which has the characteristics of a gene product isolated from a naturally occurring source. The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring. A wild-type gene is that which is most frequently observed in a population and is thus arbitrarily designated the “normal” or “wild-type” form of the gene. In contrast, the term “modified” or “mutant” when made in reference to a gene or to a gene product refers, respectively, to a gene or to a gene product which displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “antisense” or “antigenome” refers to a nucleotide sequence whose sequence of nucleotide residues is in reverse 5′ to 3′ orientation in relation to the sequence of nucleotide residues in a sense strand. A “sense strand” of a DNA duplex refers to a strand in a DNA duplex which is transcribed by a cell in its natural state into a “sense mRNA.” Thus an “antisense” sequence is a sequence having the same sequence as the non-coding strand in a DNA duplex.

The term “isolated” refers to a biological material, such as a virus, a nucleic acid or a protein, which is substantially free from components that normally accompany or interact with it in its naturally occurring environment. The isolated material optionally comprises material not found with the material in its natural environment, e.g., a cell. For example, if the material is in its natural environment, such as a cell, the material has been placed at a location in the cell (e.g., genome or genetic element) not native to a material found in that environment. For example, a naturally occurring nucleic acid (e.g., a coding sequence, a promoter, an enhancer, etc.) becomes isolated if it is introduced by non-naturally occurring means to a locus of the genome (e.g., a vector, such as a plasmid or virus vector, or amplicon) not native to that nucleic acid. Such nucleic acids are also referred to as “heterologous” nucleic acids. An isolated virus, for example, is in an environment (e.g., a cell culture system, or purified from cell culture) other than the native environment of wild-type virus (e.g., the nasopharynx of an infected individual).

An “immunologically effective amount” of a virus or attenuated virus is an amount sufficient to enhance an individual's (e.g., a human's) own immune response against a subsequent exposure to the agent. Levels of induced immunity can be monitored, e.g., by measuring amounts of neutralizing secretory and/or serum antibodies, e.g., by plaque neutralization, complement fixation, enzyme-linked immunosorbent, or microneutralization assay.

A “protective immune response” against a virus refers to an immune response exhibited by an individual (e.g., a human) that is protective against serious lower respiratory tract disease (e.g., pneumonia and/or bronchiolitis) when the individual is subsequently exposed to and/or infected with wild-type virus.

### RSV

Naturally occurring RSV particles typically contain a viral genome within a helical nucleocapsid which is surrounded by matrix proteins and an envelope containing glycoproteins. The genome of human wild-type RSV encodes the proteins, NS1, NS2, N, P, M, SH, G, F, M2-1, M2-2, and L. G, F, and SH are glycoproteins. RSV polymerase activity consists of the large protein (L) and phosphoprotein (P). The viral M2-1 protein is used during transcription and is likely to be a component of the transcriptase complex. The viral N protein is used to encapsidate the nascent RNA during replication.

The genome is transcribed and replicated in the cytoplasm of a host cell. Host-cell transcription typically results in synthesis of ten methylated and polyadenylated mRNAs. The antigenome is positive-sense RNA complement of the genome produced during replication, which in turn acts as a template for genome synthesis. The viral genes are flanked by conserved gene-start (GS) and gene-end (GE) sequences. At the 3′ and 5′ ends of the genome are leader and trailer nucleotides. The wild type leader sequence contains a promoter at the 3′ end. When the viral polymerase reaches a GE signal, the polymerase polyadenylates and releases the mRNA and reinitiates RNA synthesis at the next GS signal. The L-P complex is believed to be responsible for recognition of the promoter, RNA synthesis, capping and methylation of the 5′ termini of the mRNAs and polyadenylation of their 3′ ends. It is believed that the polymerase sometimes dissociates from the gene at the junctions. Because the polymerase initiates transcription at the 3′ end of the genome, this results in a gradient of expression, with the genes at the 3′ end of the genome being transcribed more frequently than those at the 5′ end.

To replicate the genome, the polymerase does not respond to the cis-acting GE and GS signals and generates positive-sense RNA complement of the genome, the antigenome. At the 3′ end of the antigenome is the complement of the trailer, which contains a promoter. The polymerase uses this promoter to generate genome-sense RNA. Unlike mRNA, which is released as naked RNA, the antigenome and genome RNAs are encapsidated with virus nucleoprotein (N) as they are synthesized.

After translation of viral mRNAs, a full-length (+) antigenomic RNA is produced as a template for replication of the (−) RNA genome. Infectious recombinant RSV (rRSV) particles may be recovered from transfected plasmids. Co-expression of RSV N, P, L, and M2-1 proteins as well as the full-length antigenomic RNA is sufficient for RSV replication. See Collins et al., (1995) Proc Natl Acad Sci USA. 92(25):11563-11567 and U.S. Pat. No. 6,790,449.

### Chimeric Proteins

In certain embodiments, the disclosure relates to chimeric proteins comprising at least a portion of an ectodomain from one virus, and the cytoplasmic tail of a second virus. In certain embodiments, the chimeric protein further comprises a transmembrane domain from the first or the second virus. For example, in certain embodiments, at least a portion of an ectodomain and optionally a transmembrane domain is derived from the HA protein of Orthomyxoviridae (e.g., influenza virus); the Env protein of Retroviridae; the F and/or HN proteins of Paramyxoviridae (e.g., parainfluenza, measles and mumps viruses); the S protein of Coronaviridae (e.g., SARS-CoV-2); the GP protein of Filoviridae; the GP and/or SSP proteins of Arenaviridae; the E1/E2 protein of Togaviridae; the E (e.g., in TBEV) or E1/E2 (e.g., in HCV) protein of Flaviviridae; the GN/GC protein of Bunyaviridiae; the G protein of Rhabdoviridae (e.g., VSV and rabies virus); the gB, gD, and/or gH/L protein of Herpesviridae; one or more of a complex of 8 proteins in poxviridae; and the S and/or L protein of Hepadnaviridae. In certain embodiments, the cytoplasmic tail is derived from the HA protein of Orthomyxoviridae (e.g., influenza virus); the Env protein of Retroviridae; the F and/or HN proteins of Paramyxoviridae (e.g., parainfluenza, measles and mumps viruses); the S protein of Coronaviridae (e.g., SARS-CoV-2); the GP protein of Filoviridae; the GP and/or SSP proteins of Arenaviridae; the E1/E2 protein of Togaviridae; the E (e.g., in TBEV) or E1/E2 (e.g., in HCV) protein of Flaviviridae; the GN/GC protein of Bunyaviridiae; the G protein of Rhabdoviridae (e.g., VSV and rabies virus); the gB, gD, and/or gH/L protein of Herpesviridae; one or more of a complex of 8 proteins in poxviridae; and the S and/or L protein of Hepadnaviridae.

For example, in certain embodiments, the disclosure provides a chimeric protein comprising a non-RSV fusion protein and at least a portion of an RSV F protein, as well as nucleic acids encoding the chimeric protein. In certain embodiments, the disclosure contemplates recombinant vectors comprising nucleic acids encoding these proteins and cells comprising said vectors. In certain embodiments, the vector comprises a selectable marker or reporter gene.

In certain embodiments, the disclosure relates to a chimeric protein comprising an ectodomain of a non-RSV fusion protein and an RSV F protein cytoplasmic tail. In certain embodiments, the chimeric protein further comprises a transmembrane domain of a non-RSV fusion protein or an RSV F protein. In certain embodiments, the non-RSV fusion protein is the SARS-CoV-2 spike protein.

In certain embodiments, the disclosure relates to a chimeric protein comprising an ectodomain of a first non-RSV fusion protein (e.g., a coronavirus spike protein) and cytoplasmic tail from a second non-RSV fusion protein, e.g., the HA protein of Orthomyxoviridae (e.g., influenza); the F and/or HN proteins of Paramyxoviridae (e.g., parainfluenza, measles, and mumps); the S protein of Coronaviridae (e.g., SARS-CoV-2); the GP protein of Filoviridae; the GP and/or SSP proteins of Arenaviridae; the E1/E2 protein of Togaviridae; the E (e.g., in TBEV) or E1/E2 (e.g., in HCV) protein of Flaviviridae; the GN/GC protein of Bunyaviridiae; the G protein of Rhabdoviridae; the gB, gD, and/or gH/L protein of Herpesviridae; one or more of a complex of 8 proteins in poxviridae; and the S and/or L protein of Hepadnaviridae. In certain embodiments, the chimeric protein further comprises a transmembrane domain from the first or the second non-RSV fusion protein. In certain embodiments, the first non-RSV fusion protein is the SARS-CoV-2 spike protein.

In certain embodiments, the disclosure relates to a chimeric protein comprising (1) an ectodomain and optionally a transmembrane domain of a SARS-CoV-2 spike protein and (2) a cytoplasmic tail of an influenza virus HA protein, a parainfluenza virus F or HN protein, a measles virus F or HN protein, a mumps virus F or HN protein, a vesicular stomatitis virus (VSV) G protein, or a rabies virus G protein. In certain embodiments, the chimeric protein further comprises a transmembrane domain of the influenza virus, parainfluenza virus, measles virus, mumps virus, vesicular stomatitis virus (VSV), or rabies virus. Sequences of transmembrane and cytoplasmic domains of influenza virus, parainfluenza virus, measles virus, mumps virus, vesicular stomatitis virus (VSV), and rabies virus are known in the art. Exemplary cytoplasmic tail sequences for the foregoing are provided in TABLE 1. Other contemplated cytoplasmic tail sequences include those having at least about 80%, at least about 85%, at least about 90%, at least about 95%, at least about 96%, at least about 97% at least about 98%, or at least about 99% sequence identity to the cytoplasmic tail sequences in TABLE 1.

TABLE 1
Cytoplasmic Tail Sequences

| | SEQ | |
|---|---|---|
| Viral | ID | |
| Protein | NO | Cytoplasmic Tail Sequences |
| Influenza | 116 | X₁GX₂X₃X₄CX₅ICI; where X₁ is N or K; X₂ is S or N; X₃ is L, T, M, |
| virus HA | | or C; X₄ is Q or R; X₅ is R, n or T |
| protein | 117 | NGSX₁X₂CX₃ICI; where X₁ is L, C or M. X₂ is Q or R; X₃ is R or N |
| | 118 | X₁GNX₂RCX₃ICI; where X₁ is K, N or R, X₂ is I or M, X₃ is N, T or |
| | | Q |
| Parainfluenza | 119 | KLLTIVVANRNRMENFVYHK |
| virus F and/or | 120 | MVAEDAPVRATCRVLFRTT |
| HN protein | | |
| Measles virus | 121 | CCRGRCNKKGEQVGMSRPGLKPDLTGTSKSYVRSL |
| F and/or HN | 122 | MSPQRDRINAFYKDNPHPKGSRIVINREHLMIDR |
| protein | | |
| Mumps virus | 123 | YVATKEIRRINFKTNHINTISSSVDDLIRY |
| F and/or HN | 124 | MEPSKLFIMSDNATVAPGPVVNAAGKKTFRTCFR |
| protein | | |
| Vesicular | 125 | RVGIHLCIKLKHTKKRQIYTDIEMNRLGK |
| stomatitis | | |
| virus (VSV) | | |
| G protein | | |
| Rabies virus | 126 | MTAGAMIGLVLIFSLMTWCRRANRPESKQRSFGGTGRNVSVTS |
| G protein | | |

### Coronavirus Spike Protein and Portions Thereof for Use in a Chimeric Protein

In certain embodiments, the disclosure relates to certain desirable sequences of chimeric proteins comprising at least a portion of a coronavirus (e.g., SARS-CoV-2) spike protein and at least a portion of an RSV F protein and recombinant nucleic acids encoding the same. In certain embodiments, the disclosure contemplates recombinant vectors comprising nucleic acids encoding these polypeptides and cells comprising said vectors. In certain embodiments, the vector comprises a selectable marker or reporter gene.

In certain embodiments, the disclosure relates to a chimeric protein comprising a coronavirus (e.g., SARS-CoV-2) spike (S) protein ectodomain and transmembrane domain and an RSV F protein cytoplasmic tail.

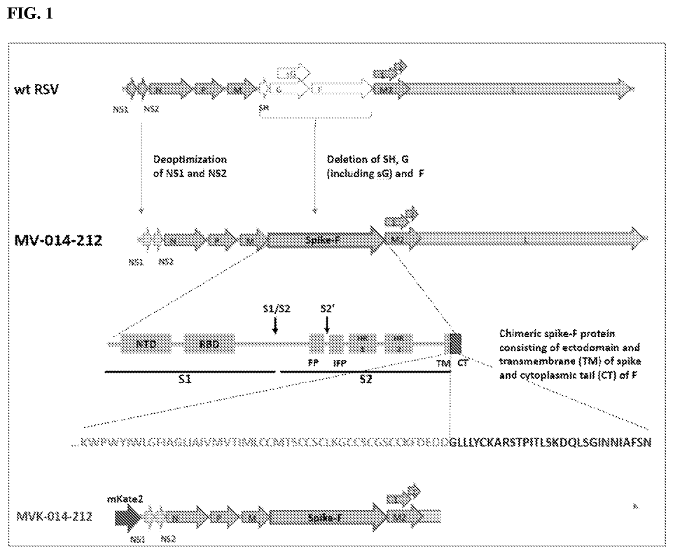

As shown in the schematic in FIG. 1 (see spike gene portion), the coronavirus spike protein comprises an S1 domain and an S2 domain separated by a furin cleavage site (S1/S2). The S1 domain contains two subdomains: an N-terminal domain (NTD) and a receptor binding domain (RBD). The S2 domain contains two heptad repeats (HR1 and HR2), an S2′ cleavage site, and a CD26-interaction domain (“CD”), a fusion peptide (FP), and a transmembrane domain (TM).

In certain embodiments, the coronavirus spike protein comprises

(SEQ ID NO: 23)
MFVFLVLLPLVSSQCVNLTTRTQLPPAYTNSFTRGVYYPDKVFRSSVLHSTQDLFLPFFSNVTWFHA
IHVSGTNGTKRFDNPVLPFNDGVYFASTEKSNIIRGWIFGTTLDSKTQSLLIVNNATNVVIKVCEFQ
FCNDPFLGVYYHKNNKSWMESEFRVYSSANNCTFEYVSQPFLMDLEGKQGNFKNLREFVFKNIDGYF
KIYSKHTPINLVRDLPQGFSALEPLVDLPIGINITRFQTLLALHRSYLTPGDSSSGWTAGAAAYYVG
YLQPRTFLLKYNENGTITDAVDCALDPLSETKCTLKSFTVEKGIYQTSNFRVQPTESIVRFPNITNL
CPFGEVFNATRFASVYAWNRKRISNCVADYSVLYNSASFSTFKCYGVSPTKLNDLCFTNVYADSFVI
RGDEVRQIAPGQTGKIADYNYKLPDDFTGCVIAWNSNNLDSKVGGNYNYLYRLFRKSNLKPFERDIS
TETYQAGSTPCNGVEGFNCYFPLQSYGFQPTNGVGYQPYRVVVLSFELLHAPATVCGPKKSTNLVKN
KCVNFNFNGLTGTGVLTESNKKFLPFQQFGRDIADTTDAVRDPQTLEILDITPCSFGGVSVITPGTN
TSNQVAVLYQDVNCTEVPVAIHADQLTPTWRVYSTGSNVFQTRAGCLIGAEHVNNSYECDIPIGAGI
CASYQTQTNSPRRARSVASQSIIAYTMSLGAENSVAYSNNSIAIPTNFTISVTTEILPVSMTKTSVD
ILPDPSKPSKRSFIEDLLFNKVTLADAGFIKQYGDCLGDIAARDLICAQKFNGLTVLPPLLTDEMIA
QYTSALLAGTITSGWTFGAGAALQIPFAMQMAYRFNGIGVTQNVLYENQKLIANQFNSAIGKIQDSL
SSTASALGKLQDVVNQNAQALNTLVKQLSSNFGAISSVLNDILSRLDKVEAEVQIDRLITGRLQSLQ
TYVTQQLIRAAEIRASANLAATKMSECVLGQSKRVDFCGKGYHLMSFPQSAPHGVVFLHVTYVPAQE
KNFTTAPAICHDGKAHFPREGVFVSNGTHWFVTQRNFYEPQIITTDNTFVSGNCDVVIGIVNNTVYD
PLQPELDSFKEELDKYFKNHTSPDVDLGDISGINASVVNIQKEIDRLNEVAKNLNESLIDLQELGKY
EQYIKWPWYIWLGFIAGLIAIVMVTIMLCCMTSCCSCLKGCCSCGSCCKFDEDDSEPVLKGVKLHYT,

or a portion or variant thereof.

In certain embodiments, the portion of a coronavirus spike protein comprises amino acids 1-1210 of SEQ ID NO: 23, amino acids 1-1254 of SEQ ID NO: 23, 1-1241 of SEQ ID NO: 23, 1-1240 of SEQ ID NO: 23, or 1-1260 of SEQ ID NO: 23.

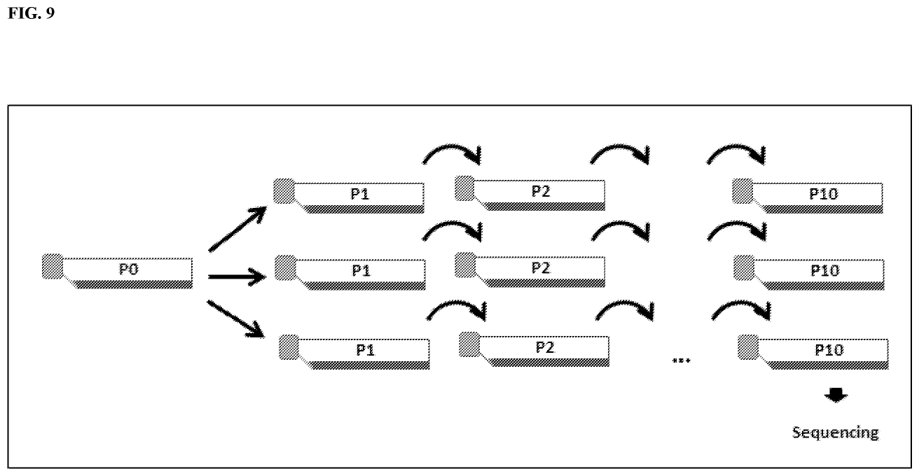

In certain embodiments, the portion of a coronavirus spike protein comprises a deletion of the furin cleavage site (PRRA (SEQ ID NO: 137)) (see amino acids 681-684 of SEQ ID NO: 23 and schematic at FIG. 9), or a mutation in the furin cleavage site (e.g., R682Q which changes the furin cleavage site from PRRA (SEQ ID NO: 137) to PQRA (SEQ ID NO: 138)). In certain embodiments, the portion of the coronavirus spike protein comprises a deletion of the amino acid P, a deletion of one of the two R amino acids, a deletion of the amino acid A, a deletion of the amino acids PR, RR, RA, PRR, or RRA of the furin cleave site. In certain embodiments, the portion of the coronavirus spike protein comprises a substitution of the amino acid P, a substitution of one or both of the two R amino acids, a substitution of the amino acid A, or any combination thereof. In certain embodiments, an amino acid of the furin cleavage site is substituted with the amino acid Q.

In certain embodiments, the portion of a coronavirus spike protein comprises one or more amino acid substitutions at positions corresponding to L5, S13, L18, T19, T20, P26, A67, D80, T95, D138, G142, W152, E154, F157, R158, R190, D215, D253, R246, K417, L452, L453, S477, T478, E484, N501, F565, A570, D614, H655, Q677, P681, A701, T716, T791, T859, F888, D950, S982, T1027I, Q1071, D1118, V1176, and/or a deletion of one or more of amino acids 69 and 70, 144, 156, and 157, wherein the amino acid numbering corresponds to SEQ ID NO: 23. In certain embodiments, the portion of a coronavirus spike protein comprises one or more of the following amino acid substitutions: L5F, S13I, L18F, T19R, T20N, P26S, A67V, D80A, D80G, T95I, D138Y, G142D, W152C, E154K, F157S, R158G, R190S, D215G, R246I, D253G, K417N, K417T, L452R, S477N, T478K, E484K, E484Q, N501Y, F565L, A570D, D614G, H655Y, Q677H, P681H, P681R, A701V, T716I, T791I, T859N, F888L, D950H, D950N, S982A, T1027I, Q1071H, and D1118H, Vi 176F, wherein the amino acid numbering corresponds to SEQ ID NO: 23.

In certain embodiments, the portion of a coronavirus spike protein comprises a combination of amino acid substitutions and/or deletions shown in any of the variants listed in TABLE 2.

TABLE 2

| | Amino Acid Position for | Exemplary Substitutions |
|---|---|---|
| Variant | Substitution or Deletion | and Deletions |
| B.1.1.7 | H69, V70, Y144, N501, | H69del, V70del, Y144del, |
| | A570, P681, T716, S982, | N501Y, A570D, P681H, |
| | D1118 | T716I, S982A, D1118H |
| B.1.351 | L18, D80, D215, R246, | L18F, D80A, D215G, R246I, |
| full | K417, E484, N501, A701 | K417T, E484K, N501Y, |
| | | A701V |
| B.1.351 | K417, E484, N501 | K417T, E484K, N501Y |
| partial | | |
| CAL20.C | S13, W152, L452 | S13I, W152C, L452R |
| P.1 full | L18, T20, P26, D138, | L18F, T20N, P26S, D138Y, |
| | R190, K417, E484, N501, | R190S, K417T, E484K, |
| | H655, T1027 | N501Y, H655Y, T1027I |
| P.1 partial | K417, E484, N501, | K417T, E484K, N501Y |
| B.1.526 | L5, T95, D253, S477, E484, | L5F, T95I, D253G, S477N, |
| full | D614, A701 | E484K, D614G, A701V |
| B.1.526 | T95, D253, D614 | T95I, D253G, D614G, |
| partial | | |
| B.1.526.1 | D80, Y144, F157, L452, | D80G, Y144del, F157S, |
| full | D614, T791, T859, D950 | L452R, D614G, T791I, |
| | | T859N, D950H |
| B.1.526.1 | D80, Y144, F157, L452, | D80G, Y144del, F157S, |
| partial | D614, D950 | L452R, D614G, D950H |
| B1.525 | A67, H69, V70, Y144, E484, | A67V, 69/70del, Y144del, |
| | D614, Q677, F888 | E484K, D614G, Q677H, |
| | | F888L |
| P.2 full | E484, F565, D614, V1176 | E484K, F565L, D614G, |
| | | V1176F |
| P.2 partial | E484, D614, V1176 | E484K, D614G, V1176F |
| B.1.617 | L452, E484, D614 | L452R, E484Q, D614G |
| B.1.617.1 | T95, G142, E154, L452, | T95I, G142D, E154K, |
| full | E484, D614, P681, Q1071 | L452R, E484Q, D614G, |
| | | P681R, Q1071H |
| B.1.617.1 | G142, E154, L452, E484, | G142D, E154K, L452R, |
| partial | D614, P681, Q1071 | E484Q, D614G, P681R, |
| | | Q1071H |
| B.1.617.2 | T19, G142, E156, F157, | T19R, G142D, E156del, |
| full | R158, L452, T478, D614, | F157del, R158G, L452R, |
| | P681, D950 | T478K, D614G, P681R, |
| | | D950N |
| B.1.617.2 | T19, E156, F157, R158, | T19R, E156del, F157del, |
| partial | L452, T478, D614, P681, | R158G, L452R, T478K, |
| | D950 | D614G, P681R, D950N |
| B.1.617.3 | T19, G142, L452, E484, | T19R, G142D, L452R, |
| | D614, P681, D950 | E484Q, D614G, P681R, |
| | | D950N |

In certain embodiments, the chimeric protein comprises a coronavirus spike protein as described herein, or a portion thereof (e.g., a fragment thereof comprising at least about 200 amino acids, at least about 300 amino acids, at least about 400 amino acids, at least about 500 amino acids, at least about 600 amino acids, at least about 700 amino acids, at least about 800 amino acids, at least about 900 amino acids, at least about 1000 amino acids, at least about 1100 amino acids, at least about 1200 amino acids, at least about 1210 amino acids, at least about 1220 amino acids, at least about 1230 amino acids, at least about 1240 amino acids, at least about 1250 amino acids, at least about 1260 amino acids, or at least about 1270 amino acids), or a variant thereof (e.g., a coronavirus spike protein comprising at least about 80%, at least about 85%, at least about 90%, at least about 95%, at least about 96%, at least about 97%, at least about 98%, or at least about 99% sequence identity thereto). In certain embodiments, the spike protein is truncated by about 1-100 amino acids, by about 1-90 amino acids, by about 1-80 amino acids, by about 1-70 amino acids, by about 1-60 amino acids, or by about 1-50 amino acids, for example, by about 1, about 2, about 3, about 4, about 5 about 6, about 7, about 8, about 9, or about 10 amino acids.

In certain embodiments, the coronavirus spike protein is encoded by SEQ ID NO: 24 or a portion or variant thereof having at least about 80%, at least about 85%, at least about 90%, at least about 95%, at least about 96%, at least about 97%, at least about 98%, or at least about 99% sequence identity thereto.

(SEQ ID NO: 24)
ATGTTTGTTTTTCTTGTTTTATTGCCACTAGTCTCTAGTCAGTGTGTTAATCTTACAACCAGAACTC
AATTACCCCCTGCATACACTAATTCTTTCACACGTGGTGTTTATTACCCTGACAAAGTTTTCAGATC
CTCAGTTTTACATTCAACTCAGGACTTGTTCTTACCTTTCTTTTCCAATGTTACTTGGTTCCATGCT
ATACATGTCTCTGGGACCAATGGTACTAAGAGGTTTGATAACCCTGTCCTACCATTTAATGATGGTG
TTTATTTTGCTTCCACTGAGAAGTCTAACATAATAAGAGGCTGGATTTTTGGTACTACTTTAGATTC
GAAGACCCAGTCCCTACTTATTGTTAATAACGCTACTAATGTTGTTATTAAAGTCTGTGAATTTCAA
TTTTGTAATGATCCATTTTTGGGTGTTTATTACCACAAAAACAACAAAAGTTGGATGGAAAGTGAGT
TCAGAGTTTATTCTAGTGCGAATAATTGCACTTTTGAATATGTCTCTCAGCCTTTTCTTATGGACCT
TGAAGGAAAACAGGGTAATTTCAAAAATCTTAGGGAATTTGTGTTTAAGAATATTGATGGTTATTTT
AAAATATATTCTAAGCACACGCCTATTAATTTAGTGCGTGATCTCCCTCAGGGTTTTTCGGCTTTAG
AACCATTGGTAGATTTGCCAATAGGTATTAACATCACTAGGTTTCAAACTTTACTTGCTTTACATAG
AAGTTATTTGACTCCTGGTGATTCTTCTTCAGGTTGGACAGCTGGTGCTGCAGCTTATTATGTGGGT
TATCTTCAACCTAGGACTTTTCTATTAAAATATAATGAAAATGGAACCATTACAGATGCTGTAGACT
GTGCACTTGACCCTCTCTCAGAAACAAAGTGTACGTTGAAATCCTTCACTGTAGAAAAAGGAATCTA
TCAAACTTCTAACTTTAGAGTCCAACCAACAGAATCTATTGTTAGATTTCCTAATATTACAAACTTG
TGCCCTTTTGGTGAAGTTTTTAACGCCACCAGATTTGCATCTGTTTATGCTTGGAACAGGAAGAGAA
TCAGCAACTGTGTTGCTGATTATTCTGTCCTATATAATTCCGCATCATTTTCCACTTTTAAGTGTTA
TGGAGTGTCTCCTACTAAATTAAATGATCTCTGCTTTACTAATGTCTATGCAGATTCATTTGTAATT
AGAGGTGATGAAGTCAGACAAATCGCTCCAGGGCAAACTGGAAAGATTGCTGATTATAATTATAAAT
TACCAGATGATTTTACAGGCTGCGTTATAGCTTGGAATTCTAACAATCTTGATTCTAAGGTTGGTGG
TAATTATAATTACCTGTATAGATTGTTTAGGAAGTCTAATCTCAAACCTTTTGAGAGAGATATTTCA
ACTGAAATCTATCAGGCCGGTAGCACACCTTGTAATGGTGTTGAAGGTTTTAATTGTTACTTTCCTT
TACAATCATATGGTTTCCAACCCACTAATGGTGTTGGTTACCAACCATACAGAGTAGTAGTACTTTC
TTTTGAACTTCTACATGCACCAGCAACTGTTTGTGGACCTAAAAAGTCTACTAATTTGGTTAAAAAC
AAATGTGTCAATTTCAACTTCAATGGTTTAACAGGCACAGGTGTTCTTACTGAGTCTAACAAAAAGT
TTCTGCCTTTCCAACAATTTGGCAGAGACATTGCTGACACTACTGATGCTGTCCGTGATCCACAGAC
ACTTGAGATTCTTGACATTACACCATGTTCTTTTGGTGGTGTCAGTGTTATAACACCAGGAACAAAT
ACTTCTAACCAGGTTGCTGTTCTTTATCAGGATGTTAACTGCACAGAAGTCCCTGTTGCTATTCATG
CAGATCAACTTACTCCTACTTGGCGTGTTTATTCTACAGGTTCTAATGTTTTTCAAACACGTGCAGG
CTGTTTAATAGGGGCTGAACATGTCAACAACTCATATGAGTGTGACATACCCATTGGTGCAGGTATA
TGCGCTAGTTATCAGACTCAGACTAATTCTCCTCGGCGGGCACGTAGTGTAGCTAGTCAATCCATCA
TTGCCTACACTATGTCACTTGGTGCAGAAAATTCAGTTGCTTACTCTAATAACTCTATTGCCATACC
CACAAATTTTACTATTAGTGTTACCACAGAAATTCTACCAGTGTCTATGACCAAGACATCAGTAGAT
TGTACAATGTACATTTGTGGTGATTCAACTGAATGCAGCAATCTTTTGTTGCAATATGGCAGTTTTT
GTACACAATTAAACCGTGCTTTAACTGGAATAGCTGTTGAACAAGACAAAAACACCCAAGAAGTTTT
TGCACAAGTCAAACAAATTTACAAAACACCACCAATTAAAGATTTTGGTGGTTTTAATTTTTCACAA
ATATTACCAGATCCATCAAAACCAAGCAAGAGGTCATTTATTGAAGATCTACTTTTCAACAAAGTGA
CACTTGCAGATGCTGGCTTCATCAAACAATATGGTGATTGCCTTGGTGATATTGCTGCTAGAGACCT
CATTTGTGCACAAAAGTTTAACGGCCTTACTGTTTTGCCACCTTTGCTCACAGATGAAATGATTGCT
CAATACACTTCTGCACTGTTAGCGGGTACAATCACTTCTGGTTGGACCTTTGGTGCAGGTGCTGCAT
TACAAATACCATTTGCTATGCAAATGGCTTATAGGTTTAATGGTATTGGAGTTACACAGAATGTTCT
CTATGAGAACCAAAAATTGATTGCCAACCAATTTAATAGTGCTATTGGCAAAATTCAAGACTCACTT
TCTTCCACAGCAAGTGCACTTGGAAAACTTCAAGATGTGGTCAACCAAAATGCACAAGCTTTAAACA
CGCTTGTTAAACAACTTAGCTCCAATTTTGGTGCAATTTCAAGTGTTTTAAATGATATCCTTTCACG
TCTTGACAAAGTTGAGGCTGAAGTGCAAATTGATAGGTTGATCACAGGCAGACTTCAAAGTTTGCAG
ACATATGTGACTCAACAATTAATTAGAGCTGCAGAAATCAGAGCTTCTGCTAATCTTGCTGCTACTA
AAATGTCAGAGTGTGTACTTGGACAATCAAAAAGAGTTGATTTTTGTGGAAAGGGCTATCATCTTAT
GTCCTTCCCTCAGTCAGCACCTCATGGTGTAGTCTTCTTGCATGTGACTTATGTCCCTGCACAAGAA
AAGAACTTCACAACTGCTCCTGCCATTTGTCATGATGGAAAAGCACACTTTCCTCGTGAAGGTGTCT
TTGTTTCAAATGGCACACACTGGTTTGTAACACAAAGGAATTTTTATGAACCACAAATCATTACTAC
AGACAACACATTTGTGTCTGGTAACTGTGATGTTGTAATAGGAATTGTCAACAACACAGTTTATGAT
CCTTTGCAACCTGAATTAGACTCATTCAAGGAGGAGTTAGATAAATATTTTAAGAATCATACATCAC
CAGATGTTGATTTAGGTGACATCTCTGGCATTAATGCTTCAGTTGTAAACATTCAAAAAGAAATTGA
CCGCCTCAATGAGGTTGCCAAGAATTTAAATGAATCTCTCATCGATCTCCAAGAACTTGGAAAGTAT
GAGCAGTATATAAAATGGCCATGGTACATTTGGCTAGGTTTTATAGCTGGCTTGATTGCCATAGTAA
TGGTGACAATTATGCTTTGCTGTATGACCAGTTGCTGTAGTTGTCTCAAGGGCTGTTGTTCTTGTGG
ATCCTGCTGCAAATTTGATGAAGACGACTCTGAGCCAGTGCTCAAAGGAGTCAAATTACATTACACA
TAA

### RSV F Protein and Portions Thereof for Use in a Chimeric Protein

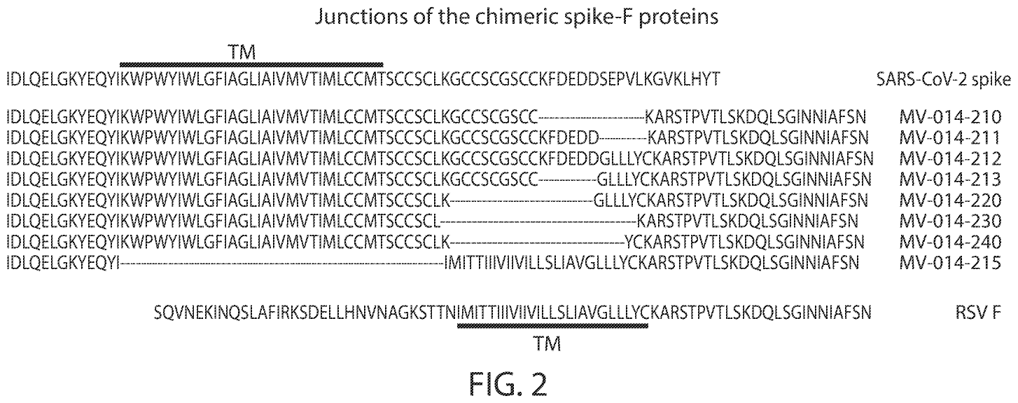

In certain embodiments, the chimeric protein comprises an RSV cytoplasmic tail (CT) domain or a portion thereof. The location and structure of the RSV cytoplasmic tail (CT) domain of the F protein is known in the art (see, e.g., Baviskar et al. (2013) J Virol 87(19): 10730-10741). In certain embodiments, and as commonly used in the art, the term RSV cytoplasmic tail (CT) domain of the F protein refers to the sequence KARSTPVTLSKDQLSGINNIAFSN (SEQ ID NO: 25) or KARSTPITLSKDQLSGINNIAFSN (SEQ ID NO: 26) (see, e.g., FIG. 2).

In certain embodiments, a portion of an RSV F protein cytoplasmic tail (CT) refers to a fragment of an RSV F protein CT comprising at least about 15 amino acids, at least about 20 amino acids, at least about 21 amino acids, at least about 22 amino acids, or at least about 23 amino acids of SEQ ID NO: 25 or 26 or a sequence comprising at least about 80%, at least about 85%, at least about 90%, at least about 95%, at least about 96%, at least about 97%, at least about 98%, or at least about 99% sequence identity to SEQ ID NO: 25 or 26. In certain embodiments, the RSV CT domain is truncated at the N- and/or C-terminus by about 1-15 amino acids, by about 1-10 amino acids, by about 1-5 amino acids, by about 1-3 amino acids, by about 5-15 amino acids, or by about 5-10 amino acids, for example, by about 1, about 2, about 3, about 4, about 5 about 6, about 7, about 8, about 9, or about 10 amino acids.

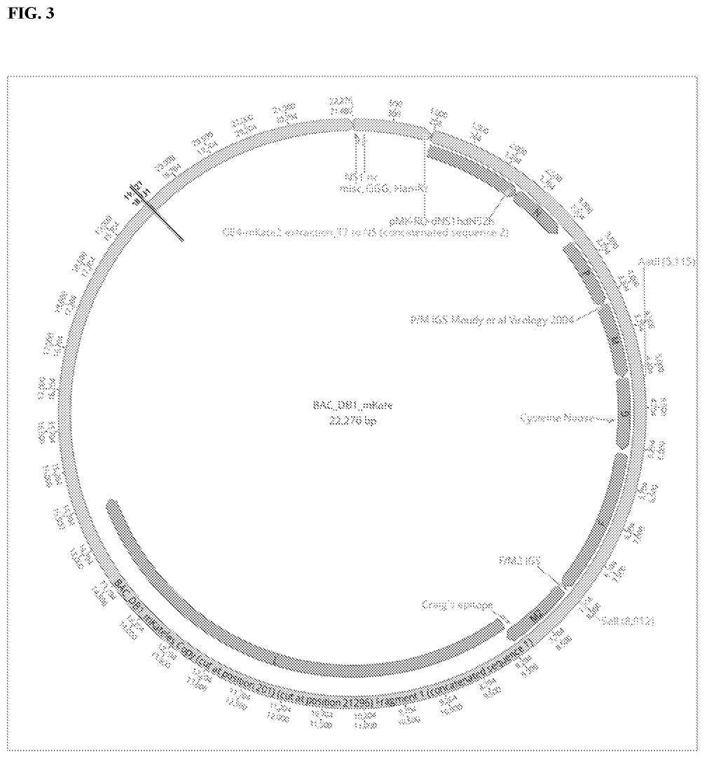

In certain embodiments, the chimeric protein comprises an RSV F protein cytoplasmic tail (CT) domain and an RSV transmembrane (TM) domain or a portion thereof. The location and structure of the RSV transmembrane domain (TM) is known in the art (see, e.g., Collins et al. (1984) PNAS 81:7683-7687 at FIG. 3) and can include the sequence IMITTIIIVIIVILLSLIAVGLLLYC (SEQ ID NO: 27) or IMIITAIIIVIIVVLLSLIAIGLLLYC (SEQ ID NO: 28). In certain embodiments, the chimeric protein comprises a portion of the RSV transmembrane (TM) domain (e.g., a fragment of an RSV transmembrane (TM) domain comprising at least about 15 amino acids, at least about 20 amino acids, at least about 21 amino acids, at least about 22 amino acids, at least about 23 amino acids, at least about 24 amino acids or at least about 25 amino acids of SEQ ID NO: 27 or 28, or a sequence comprising at least about 80%, at least about 85%, at least about 90%, at least about 95%, at least about 96%, at least about 97%, at least about 98%, or at least about 99% sequence identity to SEQ ID NO: 27 or 28). In certain embodiments, the RSV TM domain is truncated at the N- and/or C-terminus of SEQ ID NO: 27 or 28 by about 1-15 amino acids, by about 1-10 amino acids, by about 1-5 amino acids, by about 1-3 amino acids, by about 5-15 amino acids, or by about 5-10 amino acids, for example, by about 1, about 2, about 3, about 4, about 5 about 6, about 7, about 8, about 9, or about 10 amino acids.

In certain embodiments, the chimeric protein comprises at least a portion of an RSV F protein sequence that is N-terminal to the transmembrane (TM) domain of the RSV F protein, for example, GKSTTN (SEQ ID NO: 29). Accordingly, in certain embodiments, the chimeric protein comprises at least a portion of an RSV F protein sequence selected from GKSTTNIMITTIIIVIIVILLSLIAVGLLLYCKARSTPVTLSKDQLSGINNIAFSN (SEQ ID NO: 30) and GKSTTNIMITAIIIVIIVVLLSLIAIGLLLYCKARSTPITLSKDQLSGINNIAFSN (SEQ ID NO: 31) or a portion of either of the foregoing. For example, a portion of an RSV F protein sequence can include the sequence GLLLYCKARSTPVTLSKDQLSGINNIAFSN (SEQ ID NO: 32), YCKARSTPVTLSKDQLSGINNIAFSN (SEQ ID NO: 33), CKARSTPVTLSKDQLSGINNIAFSN (SEQ ID NO: 34), KARSTPVTLSKDQLSGINNIAFSN (SEQ ID NO: 35), ARSTPVTLSKDQLSGINNIAFSN (SEQ ID NO: 36), GLLLYCKARSTPITLSKDQLSGINNIAFSN (SEQ ID NO: 37), YCKARSTPITLSKDQLSGINNIAFSN (SEQ ID NO: 38), CKARSTPITLSKDQLSGINNIAFSN (SEQ ID NO: 39), KARSTPITLSKDQLSGINNIAFSN (SEQ ID NO: 40), ARSTPITLSKDQLSGINNIAFSN (SEQ ID NO: 41), or a portion of any of the foregoing (e.g., a fragment of any of the foregoing comprising at least about 15 amino acids, at least about 20 amino acids, at least about 21 amino acids, at least about 22 amino acids, at least about 23 amino acids, at least about 24 amino acids, at least about 25 amino acids, at least about 26 amino acids, at least about 27 amino acids, at least about 28 amino acids, or at least about 29 amino acids or a sequence comprising at least about 80%, at least about 85%, at least about 90%, at least about 95%, at least about 96%, at least about 97%, at least about 98%, or at least about 99% sequence identity thereto). In certain embodiments, the RSV CT domain is truncated at the N- or C-terminus by about 1-15 amino acids, by about 1-10 amino acids, by about 1-5 amino acids, by about 1-3 amino acids, by about 5-15 amino acids, or by about 5-10 amino acids, for example, by about 1, about 2, about 3, about 4, about 5 about 6, about 7, about 8, about 9, or about 10 amino acids.

In certain embodiments, the portion of the F protein used in the chimeric coronavirus S protein-RSV F protein is from the A2 RSV strain. In certain embodiments, the more thermally stable F protein from the RSV line 19 strain is used. Without wishing to be bound by theory, use of the F protein from the RSV line 19 may be advantageous because highly potent RSV neutralizing antibodies have been induced against the pre-F conformation of the F protein, and the line 19 F protein maintains relatively high levels of pre-F on the surface of virions.

### Chimeric Coronavirus S-RSV F Proteins

In certain embodiments, the disclosure provides a chimeric coronavirus-RSV protein, comprising an N-terminal portion of a coronavirus S protein and a C-terminal portion of an RSV F protein. In certain embodiments, the N-terminal portion of the chimeric coronavirus-RSV protein comprises at least about 200 amino acids, at least about 300 amino acids, at least about 400 amino acids, at least about 500 amino acids, at least about 600 amino acids, at least about 700 amino acids, at least about 800 amino acids, at least about 900 amino acids, at least about 1000 amino acids, at least about 1100 amino acids, at least about 1200 amino acids, at least about 1210 amino acids, at least about 1220 amino acids, at least about 1230 amino acids, at least about 1240 amino acids, at least about 1250 amino acids, at least about 1260 amino acids, or at least about 1270 amino acids of a coronavirus spike protein as described herein, or a variant thereof (e.g., a coronavirus spike protein comprising at least about 80%, at least about 85%, at least about 90%, at least about 95%, at least about 96%, at least about 97%, at least about 98%, or at least about 99% sequence identity to a coronavirus spike protein described herein). In certain embodiments, N-terminal portion of the spike protein is truncated by about 1-100 amino acids, by about 1-90 amino acids, by about 1-80 amino acids, by about 1-70 amino acids, by about 1-60 amino acids, or by about 1-50 amino acids, for example, by about 1, about 2, about 3, about 4, about 5 about 6, about 7, about 8, about 9, or about 10 amino acids. In certain embodiments, the C-terminal portion of the chimeric coronavirus-RSV protein comprises from about 10 to about 100 amino acids of the C-terminal portion of an RSV F protein, from about 20 to about 50 amino acids of the C-terminal portion of an RSV F protein, from about 25 to about 50 amino acids of the C-terminal portion, from about 20 to about 40 amino acids of the C-terminal portion of an RSV F protein, from about 25 to about 40 amino acids of the C-terminal portion, from about 20 to about 30 amino acids of the C-terminal portion of an RSV F protein, from about 25 to about 30 amino acids of the C-terminal portion, or about 24 amino acids of the C-terminal portion of an RSV F protein.

In certain embodiments, the portions of the coronavirus (e.g., SARS-CoV-2) and RSV sequences used in the chimeric coronavirus-RSV protein comprise about 70% or more, about 75% or more, about 80% or more, about 85% or more, about 90% or more, about 95% or more, about 96% or more, about 97% or more, about 98% or more, or about 99% or more sequence identity to a corresponding portion of a wild-type protein.

In certain embodiments, the chimeric coronavirus-RSV protein comprises a sequence selected from the group consisting of SEQ ID NOs: 1-6, 62, 68, 74, 80, 86, 92, 98, and 110, or a protein comprising about 80% or more, about 85% or more, about 90% or more, about 95% or more, about 96% or more, about 97% or more, about 98% or more, or about 99% or more sequence identity to a protein selected from the group consisting of SEQ ID NOs:1-6, 62, 68, 74, 80, 86, 92, 98, and 110.

In certain embodiments, the chimeric coronavirus-RSV protein is encoded by a nucleic acid sequence comprising a sequence selected from the group consisting of SEQ ID NOs: 7-12, 63, 69, 75, 81, 87, 93, 99, and 111, or a nucleic acid sequence comprising about 80% or more, about 85% or more, about 90% or more, about 95% or more, about 96% or more, about 97% or more, about 98% or more, or about 99% or more sequence identity to a nucleic acid selected from the group consisting of SEQ ID NOs: 7-12, 63, 69, 75, 81, 87, 93, 99, and 111, or an RNA counterpart of any of the foregoing, or a complementary sequence of any of the foregoing.

### Chimeric RSV

Common vectors for storing RSV include plasmids and bacterial artificial chromosomes (BAC). Typically, a bacterial artificial chromosome comprises one or more genes selected from the group consisting of oriS, repE, parA, and parB genes of Factor F in operable combination with a selectable marker, e.g., a gene that provides resistance to an antibiotic. The nucleic acid sequence may be the genomic or antigenomic sequence of the virus that is optionally mutated, e.g., an RSV strain that is optionally mutated.

Cultivating RSV in E. coli bacteria may be accomplished by utilizing a bacterial artificial chromosome (BAC). A BAC vector for storing and genetically engineering RSV is reported in Stobart et al., Methods Mol Biol., 2016, 1442:141-53 and U.S. Patent Application Publication number 2012/0264217. The disclosed BAC contains the complete antigenomic sequence of respiratory syncytial virus (RSV) strain A2 except the F gene, which is the antigenomic sequence of RSV strain line 19.

Accordingly, the chimeric proteins (e.g., chimeric coronavirus-RSV proteins) disclosed herein can be stored and cultivated using a BAC, e.g., the BAC reported in Stobart et al. (2016), supra, wherein the F gene and optionally the G gene are replaced with a nucleotide sequence encoding the chimeric protein.

Along with helper plasmids, the plasmid or BAC comprising a chimeric RSV can be used in the reverse genetics system for the recovery of infectious virus. The antigenome sequence on the plasmid can be mutated prior to virus recovery to generate viruses with desired mutations.

In certain embodiments, the disclosure relates to methods of generating chimeric RSV particles (e.g., chimeric coronavirus-RSV particles) comprising inserting a vector with a BAC gene and a chimeric RSV antigenome (e.g., a coronavirus-RSV antigenome) into an isolated eukaryotic cell and inserting one or more vectors (e.g., helper plasmids) selected from the group consisting of: a vector encoding an N protein (e.g., NS1 or NS2) of RSV, a vector encoding a P protein of RSV, a vector encoding an L protein of RSV, and a vector encoding an M2-1 protein of RSV into the cell under conditions such that RSV virion is formed. In certain embodiments, the vector encoding the N protein, the P protein, the L protein, or the MS-1 protein is codon-deoptimized. Inserting a vector into a cell may occur by physically injecting, electroporating, or mixing the cell and the vector under conditions such that the vector enters the cell.

Chimeric RSV (e.g., chimeric coronavirus-RSV) is contemplated to include certain mutations, deletions, or variant combinations, such as cold-passaged (cp) non-temperature sensitive (ts) derivatives of RSV, cpRSV, such as rA2cp248/404/1030ΔSH. rA2cp248/404ΔSH contains 4 independent attenuating genetic elements: cp which is based on missense mutations in the N and L proteins that together confer the non-ts attenuation phenotype of cpRSV; ts248, a missense mutation in the L protein; ts404, a nucleotide substitution in the gene-start transcription signal of the M2 gene; and ΔSH, complete deletion of the SH gene. rA2cp248/404/1030ΔSH contains independent attenuating genetic elements: those present in rA2cp248/404ΔSH and ts1030, another missense mutation in the L protein. See Karron et al., (2005) J Infect Dis. 191(7): 1093-1104, hereby incorporated by reference.

Within certain embodiments, it is contemplated that the chimeric RSV antigenome (e.g., coronavirus-RSV antigenome) may contain deletions or mutations in nonessential genes (e.g., the G, SH, NS1, NS2, and M2-2 genes) or combinations thereof. For example, in certain embodiments, the gene SH is not present. In certain embodiments, the intergenic region between the SH gene and the G gene is not present. In certain embodiments, the gene G is not present. Without wishing to be bound by theory, it is believed that exclusion of the SH gene and intergenic region between the SH gene and the G gene may increase the transcription of the chimeric RSV F protein (e.g., chimeric coronavirus S protein/RSV F protein and to attenuate the virus in vivo.

In certain embodiments, the RSV G gene comprises a Met to Ile mutation at amino acid 48 to ablate the secreted form of the G protein. Without wishing to be bound by theory, it is believed that the secreted form of the G protein acts as an antigen decoy and is not essential for in vitro replication, so ablation of the secreted form of the G protein may be advantageous.

Due to the redundancy of the genetic code, individual amino acids are encoded by multiple sequences of codons, sometimes referred to as synonymous codons. In different species, synonymous codons are used more or less frequently, sometimes referred to as codon bias. Genetic engineering of under-represented synonymous codons into the coding sequence of a gene has been shown to result in decreased rates of protein translation without a change in the amino acid sequence of the protein. Mueller et al. report virus attenuation by changes in codon bias. See, Science, 2008, 320:1784. See also WO/2008121992, WO/2006042156, Burns et al. (2006) J Virology 80(7):3259 and Mueller et al. (2006) J Virology 80(19):9687.

Usage of codon deoptimization in RSV is reported in Meng, et al., MBio 5, e01704-01714 (2014) and U.S. Patent Application Publication number 2016/0030549. In certain embodiments, this disclosure relates to isolated nucleic acids, recombinant coronavirus-RSV with codon deoptimization, vaccines produced therefrom, and vaccination methods related thereto. In certain embodiments, the codon deoptimization includes using codons that are used less frequently in humans. In certain embodiments, the codon deoptimization is in the nonstructural genes NS1 and NS2 and optionally in a gene L.

In certain embodiments, the codon deoptimization is in the nucleic acid encoding a chimeric coronavirus-RSV protein sequence selected from the group consisting of SEQ ID NOs: 1-6, 62, 68, 74, 80, 86, 92, 98, and 110 or variants thereof.

In certain embodiments, the disclosure relates to isolated nucleic acids comprising deoptimized RSV genes (e.g., NS1 and/or NS2, and optionally the gene L) of a wild-type human RSV or variants thereof wherein the nucleotides are substituted such that a codon to produce Gly is GGT, a codon to produce Asp is GAT, a codon to produce Glu is GAA, a codon to produce His is CAT, a codon to produce Ile is ATA, a codon to produce Lys is AAA, a codon to produce Leu is CTA, a codon to produce Asn is AAT, a codon to produce Gln is CAA, a codon to produce Val is GTA, or a codon to produce Tyr is TAT, or combinations thereof. In certain embodiments, a gene in the isolated nucleic acid further comprises a combination of at least two, three, four, five, six, seven, eight nine, ten, or all of the individual codons. In certain embodiment, a gene in the isolated nucleic acid comprises at least 20, 30, 40, or 50 or more of the codons.

In certain embodiments, the disclosure relates to isolated nucleic acids comprising deoptimized RSV genes (e.g., NS1 and/or NS2 optionally the gene L) of a wild-type human RSV or variants thereof wherein the nucleotides are substituted such that a codon to produce Ala is GCG, a codon to produce Cys is TGT, a codon to produce Phe is TTT, a codon to produce Pro is CCG, a codon to produce Arg is CGT, a codon to produce Ser is TCG, or a codon to produce Thr is ACG, or combinations thereof. In certain embodiments, a gene containing the nucleic acid comprises a combination of at least two, three, four, five, six, seven, eight nine, ten, eleven, twelve, thirteen, fourteen, fifteen, sixteen, or all of the individual codons. In certain embodiments, a gene in the isolated nucleic acid further comprises at least 20, 30, 40, or 50 or more of the codons.

In certain embodiments, the codon-deoptimized NS1 gene comprises the sequence:

(SEQ ID NO: 44)
ATGGGTTCGAATTCGCTATCGATGATAAAAGTACGTCTACAAAATCTATTTGATAATGATGAAGTAG
CGCTACTAAAAATAACGTGTTATACGGATAAACTAATACATCTAACGAATGCGCTAGCGAAAGCGGT
AATACATACGATAAAACTAAATGGTATAGTATTTGTACATGTAATAACGTCGTCGGATATATGTCCG
AATAATAATATAGTAGTAAAATCGAATTTTACGACGATGCCGGTACTACAAAATGGTGGTTATATAT
GGGAAATGATGGAACTAACGCATTGTTCGCAACCGAATGGTCTACTAGATGATAATTGTGAAATAAA
ATTTTCGAAAAAACTATCGGATTCGACGATGACGAATTATATGAATCAACTATCGGAACTACTAGGT
TTTGATCTAAATCCGTAA.

In certain embodiments, the codon-deoptimized NS2 gene comprises the sequence:

(SEQ ID NO: 45)
ATGGATACGACGCATAATGATAATACGCCGCAACGTCTAATGATAACGGATATGCGTCCGCTATCGC
TAGAAACGATAATAACGTCGCTAACGCGTGATATAATAACGCATAAATTTATATATCTAATAAATCA
TGAATGTATAGTACGTAAACTAGATGAACGTCAAGCGACGTTTACGTTTCTAGTAAATTATGAAATG
AAACTACTACATAAAGTAGGTTCGACGAAATATAAAAAATATACGGAATATAATACGAAATATGGTA
CGTTTCCGATGCCGATATTTATAAATCATGATGGTTTTCTAGAATGTATAGGTATAAAACCGACGAA
ACATACGCCGATAATATATAAATATGATCTAAATCCGTAA.

Without wishing to be bound by theory, codon-deoptimization of NS1 and NS2 may be advantageous because the NS1 and NS2 proteins are known to interfere with host interferon response to infection and are non-essential for in vitro replication.

In certain embodiments, the disclosure relates to isolated nucleic acids encoding deoptimized genes for a chimeric non-RSV/RSV F protein, e.g., a chimeric coronavirus S protein and RSV F protein. A chimeric coronavirus S protein and RSV F protein can have a sequence selected from the group consisting of SEQ ID NOs: 1-6, 62, 68, 74, 80, 86, 92, 98, and 110 or variants thereof or variants thereof, wherein the nucleotides are substituted such that a codon to produce Gly is GGT, a codon to produce Asp is GAT, a codon to produce Glu is GAA, a codon to produce His is CAT, a codon to produce Ile is ATA, a codon to produce Lys is AAA, a codon to produce Leu is CTA, a codon to produce Asn is AAT, a codon to produce Gln is CAA, a codon to produce Val is GTA, or a codon to produce Tyr is TAT, or combinations thereof. In certain embodiments, a gene in the isolated nucleic acid further comprises a combination of at least two, three, four, five, six, seven, eight nine, ten, or all of the individual codons. In certain embodiment, a gene in the isolated nucleic acid comprises at least 20, 30, 40, or 50 or more of the codons.

Glenn et al. report a randomized, blinded, controlled, dose-ranging study of a respiratory syncytial virus recombinant fusion (F) nanoparticle vaccine in healthy women of childbearing age ((2016) J Infect Dis. 213(3):411-22). In certain embodiments, the disclosure relates to virus particles and virus-like particles (VLPs) that contain a chimeric protein comprising a portion of a non-RSV fusion protein (e.g., a coronavirus S protein) and a portion of an RSV F protein, e.g., a chimeric coronavirus S protein and RSV F protein comprising a sequence selected from the group consisting of SEQ ID NOs: 1-6, 62, 68, 74, 80, 86, 92, 98, and 110 or variants thereof, and one or more RSV core structural proteins as described herein sufficient to form a VLP. Virus particles are commonly used as an inactivated vaccine (or killed vaccine). RSV can be grown in culture and then killed using a method such as heat or formaldehyde. Live attenuated vaccines are typically weakened such that rate of replication and/or infection is slower.

In certain embodiments, the disclosure contemplates a chimeric RSV particle (e.g., a chimeric coronavirus-RSV particle) as a whole virus vaccine, e.g., the entire virus particle exposed to heat, chemicals, or radiation such that the genome of the chimeric RSV is non-replicative or non-infectious. In certain embodiments, the disclosure contemplates a chimeric RSV particle (e.g., a chimeric coronavirus-RSV particle) in a split virus vaccine produced by using a detergent to disrupt the virus and by purifying out the chimeric proteins disclosed herein as antigens to stimulate the immune system to mount a response to the virus.

In certain embodiments, the disclosure relates to a live attenuated chimeric RSV-SARS-CoV-2 antigenome comprising a sequence selected from the group consisting of SEQ ID NOs: 13-18, 65, 71, 77, 83, 89, 95, 101, 104-109, and 113 or a variant thereof having at least about 85% (e.g., at least about 90%, at least about 95%, at least about 96%, at least about 97%, at least about 98%, or at least about 99%) sequence identity to a sequence selected from the group consisting of SEQ ID NOs: 13-18, 65, 71, 77, 83, 89, 95, 101, 104-109, and 113, or an RNA counterpart of any of the foregoing, or a complementary sequence of any of the foregoing.

VLPs closely resemble mature virions, but they do not contain viral genomic material (i.e., viral genomic RNA). Therefore, VLPs are non-replicative in nature. In addition, VLPs can express proteins on the surface of the VLP. Moreover, since VLPs resemble intact virions and are multivalent particulate structures, VLPs can be effective in inducing neutralizing antibodies to the surface protein. VLPs can be administered repeatedly.

In certain embodiments, the disclosure contemplates VLP comprising a chimeric RSV F protein (e.g., a chimeric coronavirus S protein-RSV F protein) disclosed herein on the surface and an influenza virus matrix (M1) protein core. Quan et al. report methods of producing virus-like particles (VLPs) made-up of an influenza virus matrix (M1) protein core and RSV-F on the surface. (2011) J Infect Dis. 204(7): 987-995. One can generate recombinant baculovirus (rBVs) expressing RSV F and influenza M1 and transfect them into insect cells for production.

### METHODS OF USE

In certain embodiments, the disclosure relates to immunogenic compositions comprising an immunologically effective amount of a chimeric RSV (e.g., a chimeric coronavirus-RSV), RSV and/or non-RSV (e.g., coronavirus) polypeptide, chimeric RSV (e.g., chimeric coronavirus-RSV) particle, chimeric RSV virus-like particle (e.g., a chimeric coronavirus/RSV VLP, and/or a nucleic acid disclosed herein. In certain embodiments, the disclosure relates to methods for stimulating the immune system of an individual to produce a protective immune response against a non-RSV virus (e.g., a coronavirus such as SARS-CoV-2). In certain embodiments, an immunologically effective amount of a chimeric RSV (e.g., a chimeric coronavirus-RSV), polypeptide, and/or nucleic acid disclosed herein is administered to the individual in a physiologically acceptable carrier.

In certain embodiments, the disclosure relates to medicaments and vaccine products comprising nucleic acids disclosed herein for uses disclosed herein.

In certain embodiments, the disclosure relates to the use of nucleic acids or vectors disclosed herein for the manufacture of a medicament and vaccine products for uses disclosed herein.

The disclosure also provides the ability to analyze other types of attenuating mutations and to incorporate them into chimeric RSV (e.g., chimeric coronavirus-RSV) for vaccine or other uses. For example, a tissue culture-adapted nonpathogenic strain of pneumonia virus of mice (the murine counterpart of RSV) lacks a cytoplasmic tail of the G protein (Randhawa et al., (1995) Virology 207: 240-245). By analogy, the cytoplasmic and transmembrane domains of each of the glycoproteins, HN, G and SH, can be deleted or modified to achieve attenuation.

Other mutations for use in infectious chimeric RSV (e.g., chimeric coronavirus/RSV) of the present disclosure include mutations in cis-acting signals identified during mutational analysis of chimeric RSV minigenomes (e.g., coronavirus-RSV minigenomes). For example, insertional and deletional analysis of the leader and trailer and flanking sequences identified viral promoters and transcription signals and provided a series of mutations associated with varying degrees of reduction of RNA replication or transcription. Saturation mutagenesis (whereby each position in turn is modified to each of the nucleotide alternatives) of these cis-acting signals also has identified many mutations which reduced (or in one case increased) RNA replication or transcription. Any of these mutations can be inserted into the complete antigenome or genome as described herein. Other mutations involve replacement of the 3′ end of genome with its counterpart from antigenome, which is associated with changes in RNA replication and transcription. In addition, the intergenic regions (Collins et al., (1986) Proc. Natl. Acad. Sci. USA 83:4594-4598, incorporated herein by reference) can be shortened or lengthened or changed in sequence content, and the naturally-occurring gene overlap (Collins et al. (1987) Proc. Natl. Acad. Sci. USA 84:5134-5138, incorporated herein by reference) can be removed or changed to a different intergenic region by the methods described herein.

For vaccine use, virus produced according to the present disclosure can be used directly in vaccine formulations, or lyophilized, as desired, using lyophilization protocols well known to the artisan. Lyophilized virus is typically maintained at about 4° C. When ready for use the lyophilized virus is reconstituted in a stabilizing solution, e.g., saline or comprising SPG, Mg, and HEPES, with or without adjuvant.

Typically, the chimeric RSV vaccines (e.g., coronavirus-RSV vaccines) of the disclosure contain as an active ingredient an immunogenetically effective amount of chimeric virus produced as described herein. The modified virus may be introduced into a subject with a physiologically acceptable carrier and/or adjuvant. Useful carriers are well known in the art, and include, e.g., water, buffered water, 0.4% saline, 0.3% glycine, hyaluronic acid and the like. The resulting aqueous solutions may be packaged for use as is, or lyophilized, the lyophilized preparation being combined with a sterile solution prior to administration, as mentioned above. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, and the like. Acceptable adjuvants include incomplete Freund's adjuvant, aluminum phosphate, aluminum hydroxide, or alum, which are materials well known in the art.

In certain embodiments, a chimeric RSV vaccine (e.g., coronavirus-RSV vaccine) can be formulated in a sterile, non-adjuvanted, buffered, aqueous solution filled into polypropylene cryovials. The formulation may comprise Williams E serum-free medium, sucrose, potassium phosphate dibasic, potassium phosphate monobasic, L-glutamic acid and sodium hydroxide for pH adjustment to pH 7.9.

Upon immunization with a chimeric RSV composition (e.g., coronavirus-RSV composition) as described herein, via aerosol, droplet, oral, topical or other route, the immune system of the subject responds to the vaccine by producing antibodies specific for virus proteins, e.g., S glycoproteins. As a result of the vaccination, the subject becomes at least partially or completely immune to coronavirus infection, or resistant to developing moderate or severe coronavirus infection, particularly of the lower respiratory tract.

The subject to which the vaccines are administered can be any mammal which is susceptible to infection by a non-RSV (e.g., coronavirus, e.g., SARS-CoV-2) or a closely related virus and which subject is capable of generating a protective immune response to the antigens of the vaccinating strain. Thus, suitable subjects include humans, non-human primates, bovine, equine, swine, ovine, caprine, lagamorph, rodents, etc. Accordingly, the disclosure provides methods for creating vaccines for a variety of human and veterinary uses.

The vaccine compositions containing the chimeric RSV (e.g., coronavirus-RSV) of the disclosure are administered to a subject susceptible to or otherwise at risk of coronavirus infection to enhance the subject's own immune response capabilities. Such an amount is defined to be an “immunogenically effective dose.” In this use, the precise amounts again depend on the subject's state of health and weight, the mode of administration, the nature of the formulation. The vaccine formulations should provide a quantity of chimeric coronavirus-RSV of the disclosure sufficient to effectively protect the subject patient against serious or life-threatening infection.

The chimeric RSV (e.g., coronavirus-RSV) produced in accordance with the present disclosure can be combined with viruses of the other subgroup or strains to achieve protection against multiple non-RSV (e.g., coronavirus) subgroups or strains, or protective epitopes of these strains can be engineered into one virus as described herein. Typically, the different viruses are in admixture and administered simultaneously, but may also be administered separately. For example, as the S glycoproteins of the coronavirus subgroups differ in amino acid sequence, this similarity is the basis for a cross-protective immune response as observed in animals immunized with chimeric coronavirus-RSV or S antigen and challenged with a heterologous strain. Thus, immunization with one strain may protect against different strains of the same or different subgroup.

In some instances, it may be desirable to combine the chimeric RSV vaccines (e.g., chimeric coronavirus-RSV vaccines) of the disclosure with vaccines that induce protective responses to other agents. For example, the chimeric RSV vaccine (e.g., chimeric coronavirus-RSV vaccine) of the present disclosure can be administered simultaneously with an influenza vaccine.

Single or multiple administrations of the vaccine compositions of the disclosure can be carried out. In certain embodiments, a single dose of the vaccine compositions is sufficient to generate immunity. In certain embodiments, no adjuvant is required. Multiple, sequential administrations may be required to elicit sufficient levels of immunity. Administration may begin within the first month of life, or before, about two months of age, typically not later than six months of age, and at intervals throughout childhood, such as at two months, six months, one year and two years, as necessary to maintain sufficient levels of protection against native (wild-type) infection. Similarly, adults who are particularly susceptible to repeated or serious coronavirus infection, such as, for example, health care workers, day care providers, elder care providers, the elderly (over 55, 60, 65, 70, 75, 80, 85, or 90 years), or individuals with compromised cardiopulmonary function may require multiple immunizations to establish and/or maintain protective immune responses. Levels of induced immunity can be monitored by measuring amounts of neutralizing secretory and serum antibodies, and dosages adjusted or vaccinations repeated as necessary to maintain desired levels of protection. Further, different vaccine viruses may be advantageous for different recipient groups. For example, an engineered strain expressing an additional protein rich in T-cell epitopes may be particularly advantageous for adults rather than for infants.

Administration is typically by aerosol, nebulizer, or other topical application to the respiratory tract of the patient being treated. Recombinant chimeric RSV (e.g., chimeric coronavirus-RSV) is administered in an amount sufficient to result in the expression of therapeutic or prophylactic levels of the desired gene product. Examples of representative gene products which are administered in this method include those which encode, for example, those particularly suitable for transient expression, e.g., interleukin-2, interleukin-4, gamma-interferon, GM-CSF, G-CSF, erythropoietin, and other cytokines, glucocerebrosidase, phenylalanine hydroxylase, cystic fibrosis transmembrane conductance regulator (CFTR), hypoxanthine-guanine phosphoribosyl transferase, cytotoxins, tumor suppressor genes, antisense RNAs, and vaccine antigens.

In certain embodiments, the disclosure relates to immunogenic compositions (e.g., vaccines) comprising an immunologically effective amount of a recombinant chimeric RSV (e.g., chimeric coronavirus-RSV) of the disclosure (e.g., an attenuated live recombinant chimeric RSV or inactivated, non-replicating chimeric RSV), an immunologically effective amount of a polypeptide disclosed herein, and/or an immunologically effective amount of a nucleic acid disclosed herein.

In certain embodiments, the disclosure relates to methods for stimulating the immune system of an individual to produce a protective immune response against coronavirus. In the methods, an immunologically effective amount of a recombinant chimeric RSV (e.g., chimeric coronavirus-RSV) disclosed herein, an immunologically effective amount of a polypeptide disclosed herein, and/or an immunologically effective amount of a nucleic acid disclosed herein is administered to the individual in a physiologically acceptable carrier.

Typically, the carrier or excipient is a pharmaceutically acceptable carrier or excipient, such as sterile water, aqueous saline solution, aqueous buffered saline solutions, aqueous dextrose solutions, aqueous glycerol solutions, ethanol, or combinations thereof. The preparation of such solutions ensuring sterility, pH, isotonicity, and stability is affected according to protocols established in the art. Generally, a carrier or excipient is selected to minimize allergic and other undesirable effects, and to suit the particular route of administration, e.g., subcutaneous, intramuscular, intranasal, oral, topical, etc. The resulting aqueous solutions can e.g., be packaged for use as is or lyophilized, the lyophilized preparation being combined with a sterile solution prior to administration.

In certain embodiments, the chimeric RSV (e.g., chimeric coronavirus/RSV) or component thereof (e.g., a chimeric non-RSV/RSV fusion protein such as a chimeric coronavirus S protein-RSV F protein), is administered in a quantity sufficient to stimulate an immune response specific for one or more strains of non-RSV such as coronavirus. In other words, in certain embodiments, an immunologically effective amount of chimeric RSV (e.g., a coronavirus-RSV) or component thereof, e.g., a chimeric non-RSV/RSV fusion protein (e.g., a chimeric coronavirus S protein-RSV F protein) is administered. Preferably, administration of a chimeric RSV (e.g., a chimeric coronavirus/RSV) elicits a protective immune response. Dosages and methods for eliciting a protective anti-viral immune response, adaptable to producing a protective immune response against a non-RSV (e.g., a coronavirus) and/or RSV, are known to those of skill in the art. See, e.g., U.S. Pat. No. 5,922,326; Wright et al. (1982) Infect. Immun. 37:397-400; Kim et al. (1973) Pediatrics 52:56-63; and Wright et al. (1976) J. Pediatr. 88:931-936. For example, virus can be provided in the range of about 10³-10⁷ pfu (plaque forming units) per dose administered (e.g., 10³-10⁷ pfu, 10³-10⁶ pfu, 10³-10⁵ pfu, 10⁴-10⁷ pfu, 10⁴-10⁶ pfu, or 10⁴-10⁶ pfu per dose administered). In certain embodiments, the virus is provided in an amount of about 10³ pfu per dose administered. In certain embodiments, the virus is provided in an amount of about 10⁴ pfu per dose administered. In certain embodiments, the virus is provided in an amount of about 10⁵ pfu per dose administered. In certain embodiments, the virus is provided in an amount of about 10⁶ pfu per dose administered. In certain embodiments, the virus is provided in an amount of about 10⁷ pfu per dose administered. Typically, the dose is adjusted based on, e.g., age, physical condition, body weight, sex, diet, mode and time of administration, and other clinical factors.

The vaccine formulation can be systemically administered, e.g., by subcutaneous or intramuscular injection using a needle and syringe or a needleless injection device. The vaccine formulation can be administered intratracheally. Preferably, the vaccine formulation is administered intranasally, e.g., by drops, aerosol (e.g., large particle aerosol (greater than about 10 microns)), or spray into the upper respiratory tract. While any of the above routes of delivery results in a protective systemic immune response, intranasal administration confers the added benefit of eliciting mucosal immunity at the site of entry of the virus (i.e., may generate both mucosal and humoral immune responses). While humoral immunity (circulating antibodies) are important for preventing serious lung disease, mucosal antibodies are important for blocking infection and transmission of respiratory viruses. For intranasal administration, attenuated live virus vaccines are often preferred, e.g., an attenuated, cold adapted and/or temperature sensitive recombinant virus. Further, unlike many candidate SARS-CoV-2 vaccines in pre-clinical and clinical development, in certain embodiments, a single intranasal inoculation of a live, attenuated, replicating chimeric coronavirus/RSV vaccine as described herein can be sufficient to generate immunity. Additionally, additionally in certain embodiments, no adjuvant is present, avoiding the need for an additional formulation component and the need to evaluate adjuvant activity in clinical studies.

As an alternative or in addition to attenuated live virus vaccines, killed virus vaccines, nucleic acid vaccines, and/or polypeptide subunit vaccines, for example, can be used, as suggested by Walsh et al (1987) J. Infect. Dis. 155:1198-1204 and Murphy et al (1990) Vaccine 8:497-502.

In certain embodiments, the attenuated recombinant chimeric coronavirus-RSV is as used in a vaccine and is sufficiently attenuated such that symptoms of infection, or at least symptoms of serious infection, does not occur in most individuals immunized (or otherwise infected) with the attenuated virus—in embodiments in which viral components (e.g., the nucleic acids or polypeptides herein) are used as vaccine or immunogenic components. However, virulence is typically sufficiently abrogated such that mild or severe lower respiratory tract infections do not typically occur in the vaccinated or incidental subject.

While stimulation of a protective immune response with a single dose is preferred, additional dosages can be administered, by the same or different route, to achieve the desired prophylactic effect. In neonates and infants, for example, multiple administrations may be required to elicit sufficient levels of immunity. Administration can continue at intervals throughout childhood, as necessary to maintain sufficient levels of protection against wild-type coronavirus infection. Similarly, adults who are particularly susceptible to repeated or serious coronavirus infection, such as, for example, health care workers, day care providers, elder care providers, the elderly (over 55, 60, 65, 70, 75, 80, 85, or 90 years), and individuals with compromised cardiopulmonary function may require multiple immunizations to establish and/or maintain protective immune responses. Levels of induced immunity can be monitored, for example, by measuring amounts of virus-neutralizing secretory and serum antibodies, and dosages adjusted or vaccinations repeated as necessary to elicit and maintain desired levels of protection.

Alternatively, an immune response can be stimulated by ex vivo or in vivo targeting of dendritic cells with virus. For example, proliferating dendritic cells are exposed to viruses in a sufficient amount and for a sufficient period of time to permit capture of the coronavirus antigens by the dendritic cells. The cells are then transferred into a subject to be vaccinated by standard intravenous transplantation methods.

Optionally, the formulation for administration of the vaccine also contains one or more adjuvants for enhancing the immune response to the coronavirus antigens. Contemplated adjuvants include aluminum salts such as Alhydrogel® and Adjuphos®. Contemplated adjuvants include oil-in-water emulsions, where the oil acts as the solute in the water phase and forms isolated droplets, stabilized by emulsifying agents. In certain embodiments, emulsions contain a squalene or α-tocopherol (vitamin E) with additional emulsifying agents such as sorbitan trioleate and polysorbate-80 (PS80) as surfactants. In certain embodiments, emulsions contain a glucopyranosyl lipid A (GLA). GLA can be formulated with chimeric coronavirus-RSV, particles or chimeric coronavirus S protein-RSV F protein either alone or in a squalene-based oil-in-water stable emulsion (SE). Iyer et al. report oil-in-water adjuvants of different particle size using RSV F protein ((2015) Hum Vaccin Immunother 11(7): 1853-1864).

Suitable adjuvants include, for example: complete Freund's adjuvant, incomplete Freund's adjuvant, saponin, mineral gels such as aluminum hydroxide, surface-active substances such as lysolecithin, pluronic polyols, polyanions, peptides, oil or hydrocarbon emulsions, bacille Calmette-Guerin (BCG), Corynebacterium parvum, and the synthetic adjuvant QS-21.

If desired, prophylactic vaccine administration of chimeric coronavirus-RSV can be performed in conjunction with administration of one or more immunostimulatory molecules. Immunostimulatory molecules include various cytokines, lymphokines and chemokines with immunostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immunostimulatory molecules can be administered in the same formulation as the chimeric coronavirus-RSV or can be administered separately. Either the protein or an expression vector encoding the protein can be administered to produce an immunostimulatory effect.

Although vaccination of an individual with an chimeric coronavirus-RSV of a particular strain of a particular subgroup can induce cross-protection against viruses of different strains and/or subgroups, cross-protection can be enhanced, if desired, by vaccinating the individual with attenuated coronavirus from at least two strains, e.g., each of which represents a different subgroup. Similarly, the chimeric coronavirus-RSV vaccines can optionally be combined with vaccines that induce protective immune responses against other infectious agents.

A potential challenge for a coronavirus live attenuated vaccine is recombination (genetic instability). Natural genomic recombination is a common feature of coronaviruses and other positive-sense viruses in the Nidovirales order. In contrast, natural recombination is rare for viruses like RSV and measles virus (wild-type or vaccine strains) of the negative-sense Mononegavirales order. Furthermore, live attenuated RSV vaccines have been shown to be genetically stable (Stobart (2016), supra), likely owing to the fact that attenuating mutations are either via extensive codon deoptimization or deletion of viral genes. Accordingly, in certain embodiments, a chimeric coronavirus-RSV as described herein exhibits little to no genetic instability.

In addition, SARS coronaviruses and RSV have in common the potential risk of vaccine-associated enhanced respiratory disease (VAERD) which is associated with certain types of vaccines, for example, non-replicating (e.g. subunit) vaccine types. However, live attenuated coronavirus vaccines have not demonstrated VAERD in contrast to other vaccine technologies such fixed whole virus, subunit, and some vector vaccines. Accordingly, in certain embodiments, a chimeric coronavirus-RSV as described herein does not increase the risk of VAERD. VAERD can be measured in preclinical animal models by assessing markers of inflammation that include excessive pulmonary immune cell infiltrates, elevated Th2 inflammatory cytokine levels, and lung damage by histopathology.

In certain embodiments, a chimeric RSV (e.g., chimeric coronavirus-RSV) as described herein exhibits (1) high levels of virus-neutralizing antibodies relative to binding, non-neutralizing antibodies, and (2) T cell responses having canonical Th1 antiviral cytokines and/or do not exhibit an imbalance towards high levels of Th2 cytokines.

### EXAMPLES

The following examples are merely illustrative and are not intended to limit the scope or content of the invention in any way.

### Example 1—Construction of Chimeric Coronavirus Spike Protein—RSV Fusion Protein

A series of live attenuated vaccine candidates (attRSV-CoV-2, MV-014 series) was constructed by cloning the SARS-CoV2 spike protein (strain USA-WA1/2020) in place of the RSV G and F proteins in an attenuated RSV vector derived from MV-012-968 (FIG. 1). The RSV backbone contains the gene for the mKate2 fluorescent protein and is called DB1 Quad mKate. (See (Rostad et al., (2018) Journal of Virology 92 (6) e01568-17).)

The cytoplasmic tail of RSV F is required for RSV infectious progeny assembly (Baviskar et al. (2013) Journal of Virology 87(19), 10730-10741). Therefore, it was hypothesized that replacing F with a full length S gene would result in a non-viable virus. Accordingly, as depicted in FIG. 1, a chimeric Spike gene was created wherein the cytoplasmic tail of Spike was replaced with the cytoplasmic tail of RSV F (blue CT portion of the green S gene). The cytoplasmic tail of SARS-CoV-1 was not required for infectivity of Spike-pseudotyped virus (Broer et al. (2006) Journal of Virology 80(3), 1302-1310). However, the transmembrane and juxtamembrane regions of SARS-1 Spike are essential for assembly and entry, though the mechanisms are not fully defined (Corver et al. (2009) Virology Journal 6(1), 230; Godeke et al. (2000) Journal of Virology 74(3), 1566-1571). The amino acid sequence of the transmembrane domain of Spike fused to the cytoplasmic tail of RSV F (underlined text) is depicted at the bottom of FIG. 1.

Six constructs were designed that contained different C-terminal sequences of RSV F fused with the SARS-CoV2 spike protein ectodomain, and 1 wild-type spike construct was designed (see FIG. 2 and appendix for full sequences).

The chimeric spike-F genes were designed to contain flanking AatII and SalI sites for cloning into the BAC DB1 Quad mKate backbone (see schematic in FIG. 3 and sequence of BAC DB1 Quad mKate is SEQ ID NO: 46), replacing the DNA fragment encompassing the genes for RSV G and F proteins (nt 5,111 to nt 8015).

Inserts 210 (SEQ ID NO: 47), 220 (SEQ ID NO: 50), 230 (SEQ ID NO: 51), 240 and (SEQ ID NO: 52), and 300 (SEQ ID NO: 53) were synthesized by Genscript and inserts 211 (SEQ ID NO: 48) and 212 (SEQ ID NO: 49) were synthesized by Twist Bioscience and received as a lyophilized pellet.

The spike-F inserts and the DB1 Quad mKate vector were digested with the enzymes AatII and Sal I. The DNA corresponding to the digested spike-F inserts (˜4 kb) and DB1Quad mKate without G and F (˜20 kb) was purified from a gel and ligated using T4 DNA ligase. The product of the ligation was used to transform One-shot Stabl3 chemically competent cells (Thermo C737303). The transformants were analyzed by sequencing of the BAC DNA. The antigenomes cloned in BAC were sequenced by Genewiz using 74 primers that provided a coverage of ˜2 (on average) for the entire constructs.

Sequences of the BACs encoding the RSV-coronavirus genome vaccine candidates (with mKate2 marker) are provided in SEQ ID NOs: 54-59 (inserts 210, 211, 212, 220, 230, and 240, respectively). The sequence of the BAC encoding the RSV-coronavirus genome with wild-type coronavirus spike protein (insert 300) is at SEQ ID NO: 60. The antigenome sequences for the mKate-containing viruses contained within the BAC constructs is provided at SEQ ID NOs: 104-109 (inserts 210, 211, 212, 220, 230, and 240, respectively).

Versions of these constructs without the label protein mKate2 were constructed using restriction cloning. Specifically, the fragment of the BAC containing the gene for mKate2 was released via digestion with the enzymes KpnI (that cuts in the BAC) and AatII (that cuts inside the antigenome). DB1 Quad without mKate2 is digested with the same enzymes and the fragment without mKate2 is used to replace the fragment with mKate2 in the MV-014 constructs. Sequences of the BACs comprising the RSV-coronavirus genome vaccine candidates (without mKate2 marker) are provided in SEQ ID NOs: 131-136 (inserts 210, 211, 212, 220, 230, and 240, respectively).

MV-014 constructs without mKate2 and with inserts 210, 211, 212, 220, 230, and 240 are provided in SEQ ID NOs: 13-18, respectively, which are the antigenome sequences for the vaccine candidates.

BACs for all clones were prepared using Macherey Nagel NucleoBond Xtra BAC or Zymo Research ZymoPureII MaxiPrep kits from 500 ml overnight cultures. The obtained BAC DNA (with or without the mKate marker) was further used for virus rescue in tissue culture, as described in Example 2.

### Example 2—Viral Rescue

Vero RCB2 cells were cultivated in serum free MEM supplemented with 4 mM glutamine. Cells were seeded at 7.5×10e5/well in a 6 well dish containing 2 mL of media and incubated overnight at 37° C., 5% CO₂ in a humid incubator. The next day, the media was removed and the cell monolayer washed twice with Opti-MEM and incubated with 2 mL Opti-MEM at 37° C., 5% CO₂ in a humid incubator.

To rescue virus, Vero RCB2 cells were transfected with plasmids expressing the antigenome for DB1-Quad-mKate2 RSV or MV-014-210 (DB1-Quad-mKate2 RSV with 210 insert as described in Example 1) with helper plasmids expressing codon deoptimized RSV N, P, M2.1 and L cloned into the pXT7 vector and a plasmid expressing the T7 RNA polymerase.

Transfection mixtures were assembled for each condition by mixing 15 uL of Lipofectamine 2000CD into 250 uL Opti-MEM and incubating the mixture for 5 min. at room temperature. In a separate tube, plasmid DNA containing the antigenome of DB1-Quad-mKate2 (RSV vector) or MV-014-210 (1.5 ug) was mixed with plasmids expressing RSV-N (1 ug), RSV P (1 ug), RSV M2-1 (0.75 ug), RSV L (0.5 ug) and the T7 RNA polymerase (1.25 ug). The plasmid DNA mixtures were added to 250 uL of Opti-MEM in a 1.5 mL microfuge tube and incubated for 5 min. at room temperature. The DNA-Opti-MEM mixture was combined with the lipofectamine-Opti-MEM mixture and vortexed for 5 seconds and then incubated for 30 min at room temperature. The media in the 6 well plate was removed and the DNA-lipofectamine mixture was slowly added to the cell monolayer. The cells were incubated at room temperature for 1 h with gentle rocking. At the end of this incubation, 2 mL of Opti-MEM was added to each well and the cells were incubated overnight at 37° C., 5% CO₂ in a humid incubator.

The next day the media was removed and replaced with 2 mL of 1×MEM supplemented with 10% fetal bovine serum and 1× antibiotics.

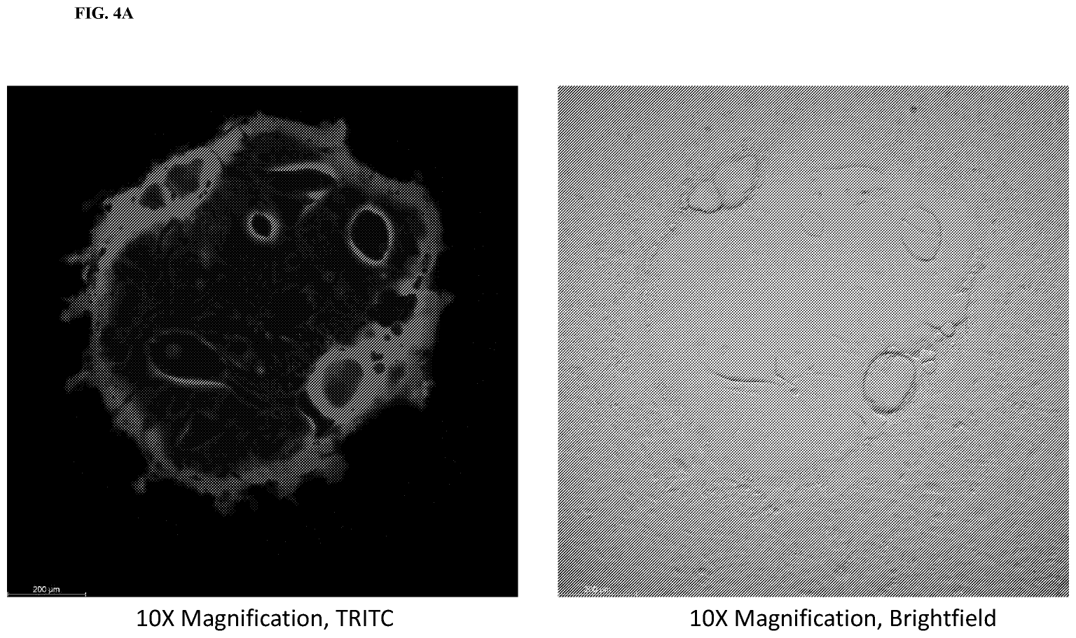

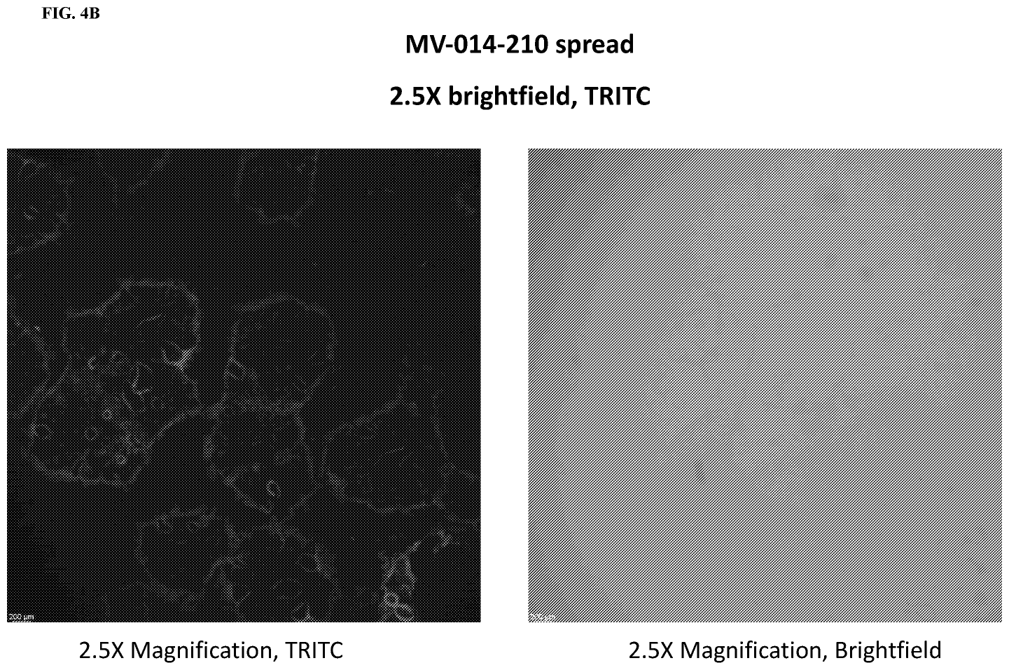

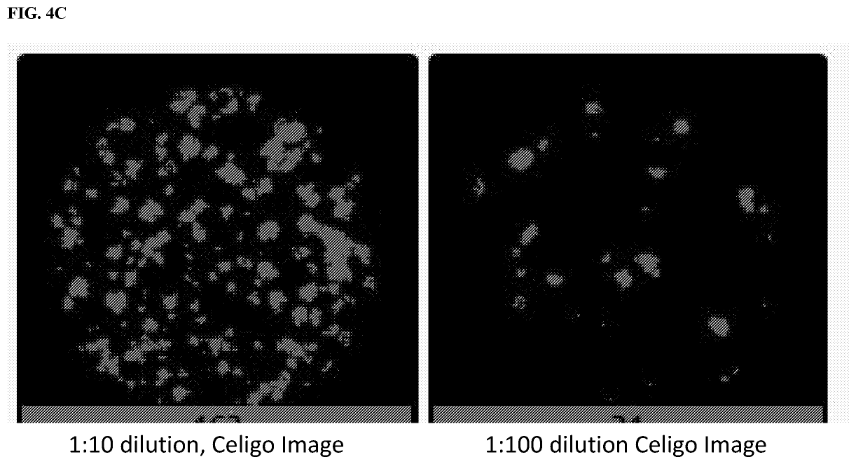

FIGS. 4 A and B provides fluorescence and brightfield images of virus foci (TRITC) and cytopathic effects (brightfield) on Vero cell monolayers that were passaged multiple times after transfection with MV-014-210 and RSV helper plasmids. Shown is a large focus at 10× magnification (FIG. 4A) and evidence for extensive replication and spread at 2.5× magnification (FIG. 4B). Fluorescence images were generated using TRITC filter set to visualize mKate2 expression. Virus stocks were prepared as cell free lysates from infected Vero cells were used to infect fresh Vero cell monolayers in a 24 well plate at different dilutions (FIG. 4C). The formation of foci after infection with cell free lysates is consistent with isolation of intact infectious particles that infect via the chimeric spike-F protein. Fluorescence images was generated using the Celigo imaging instrument set to detect mKate2 expression.

This experiment demonstrated that plasmids encoding a recombinant RSV with a chimeric coronavirus spike protein/RSV F protein are suitable for use in the preparation of a vaccine.

### Example 3—Vaccination with MV-014-212 Protects Primates from SARS-CoV-2 Challenge and Results in Specific Neutralization of Both MVK-014-212 and the B.1.351 Variant

### Design and Generation of MV-014-212 and MVK-014-212-B.1.351

MV-014-212 is a novel live attenuated, recombinant vaccine against SARS-CoV-2, based on the backbone of the human respiratory syncytial virus (RSV) (FIG. 1). The attachment and fusion proteins G and F of RSV were replaced by a chimeric protein consisting of the ectodomain and transmembrane (TM) domains of SARS-CoV-2 spike (strain USA-WA1/2020) and the cytoplasmic tail of RSV F (line19 strain). The sequence of amino acids at the junction between spike and F proteins is shown in FIG. 1. Notably, the chimeric spike/RSV F protein retains functionality as MV-014-212 growth relies on it for attachment and fusion with the host cell. Various chimeric spike constructs that differed in the junction position were assessed for growth in Vero cells (FIG. 2). In particular, a construct with the entire native SARS-CoV-2 spike was evaluated (MV-014-300, FIG. 2). While this construct could be rescued, it did not propagate efficiently in cell culture. Results of rescue experiments are shown in TABLE 3.

TABLE 3

| | | Achieved Titers ≥ |
|---|---|---|
| Vaccine candidate | Rescue | 10⁵ PFU/mL |
| MV-014-210 | Y | Y |
| MV-014-211 | Y | N.D. |
| MV-014-212 | Y | Y |
| MV-014-220 | Y | N.D. |
| MV-014-230 | N | N |
| V-014-240 | N | N |
| MV-014-300 | Y | N |

Of the constructs expressing different chimeric spike/RSV F fusion proteins, MV-014-212 was selected for further evaluation based on the ease of rescue and its ability to grow to acceptable titers for pre-clinical and clinical studies.

The RSV backbone used to generate MV-014-212 was attenuated for replication in primary cells by codon deoptimization of the genes encoding the proteins NS1 and NS2 that suppress host innate immunity (Meng et al. (2014) mBio 5(5):e01704-14). In addition, the short hydrophobic glycoprotein SH was deleted to increase transcription of downstream genes (Bukreyev 1997).

To facilitate the development of a microneutralization assay, a reporter virus derived from MV-014-212 was also constructed by inserting the gene encoding the fluorescent mKate2 protein (Hotard et al. (2012) Virology 434(1):129-36, Shchervo et al. (2009) Biocherm J. 418(3):567-74) upstream of the NS1 gene (MVK-014-212, K for mKate, FIG. 1, bottom).

SARS-CoV-2 has a high rate of mutation and new variants evolve rapidly. Recently, variant strains of SARS-CoV-2 have raised concern because they present mutations in the spike RBD suspected to lead to the loss of neutralizing epitopes and consequent evasion of immunity raised by vaccination or natural infection with the ancestral strains of SARS-CoV-2 Wuhan-1 or USA/WA2020 (identical in the spike coding region). Of note is the variant B.1.351 which carries 8 mutations in the spike protein, 3 of which reside in the RBD: K417N, E484K and N501Y (Tegally et al. (2020) Nature 592(7854):438-443). In particular, E484K was also identified through repeated passage in the presence of neutralizing sera to isolate neutralizing escape mutants (Andreano et al. (2020) bioRxiv [Preprint]. December 28:2020.12.28.424451). Several studies have shown that neutralizing antibodies elicited by the currently marketed vaccines or present in convalescent sera are less efficient at neutralizing the B.1.351 variant compared to the Wuhan-1 strain (Wang et al. (2021) Nature 592(7855):616-622; Liu et al. (2021) N Engl J Med. 2021 Apr. 15; 384(15):1466-1468; Madhi et al. (2021) N Engl J Med. 384(20):1885-1898; Wibmer et al. (2021) Nat Med. 27(4):622-625).

Accordingly, a variant of MVK-014-212, MVK-014-212-B.1.351, was generated incorporating the mutations in spike observed in the SARS-CoV-2 variant B.1.351. The variations in MVK-014-212-B.1.351 relative to MVK-014-212 are listed in TABLE 4.

TABLE 4
Mutations in the B.1.351 strain of SARS-CoV-2 relative
to the USA/WA-2020 strain used in this study.
Mutations in B.1.351
(UA-WA/2020 to B.1.351)
D80A
D215G
dLLA 214-3
K417N
E484K
N501Y
D614G
A701V

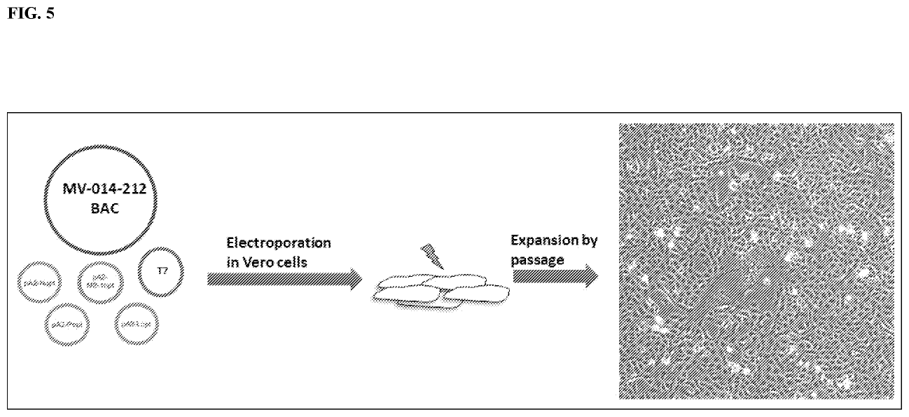

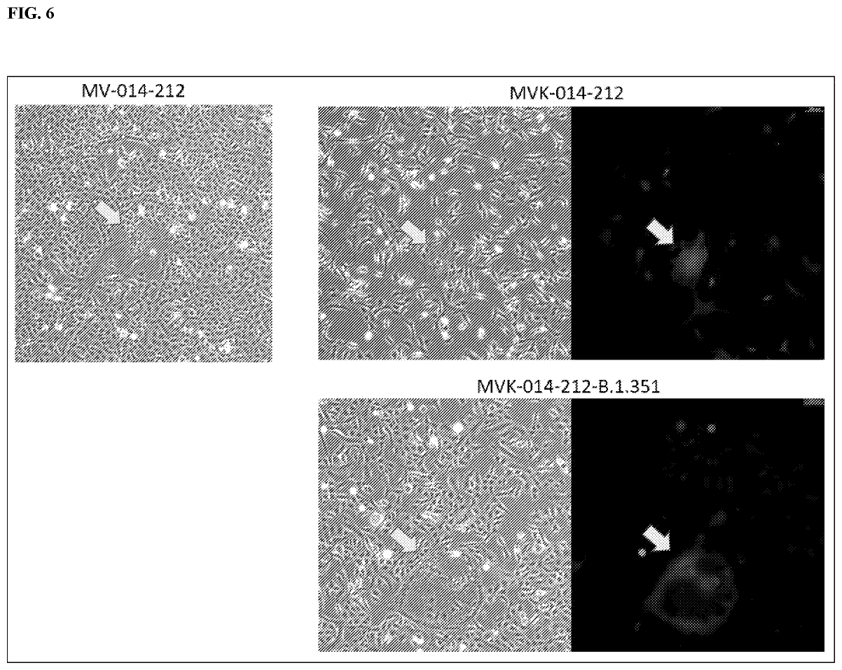

All recombinant virus constructs were electroporated into Vero cells and infectious virus was rescued and propagated for further characterization (Hotard (2012), supra). Briefly, Vero cells were electroporated with the bacterial artificial chromosome (BAC) encoding MV-014-212 (or the reporter viruses) together with helper plasmids encoding the T7 polymerase and the RSV proteins N, P, M2-1 and L, under the control of a CMV promoter (FIG. 5). During recovery from electroporation, the cells were monitored for evidence of cytopathic effect (CPE). In MV-014-212, CPE is observed as the formation of polynucleated bodies or syncytia and eventual cell detachment (FIG. 6). The electroporated cells were expanded until the CPE was extensive and the virus stock was harvested as a total cell lysate. The titers obtained for MV-014-212 and derived viruses were comparable and within the range 1-5 10⁵ PFU/mL. FIG. 6 shows micrographs taken during the rescue of MV-014-212 and MVK-014-212.

The protein sequence of the chimeric coronavirus spike/RSV F protein in MVK-014-212-B.1.351 is provided at SEQ ID NO: 62, and the nucleic acid sequence encoding the protein is provided at SEQ ID NO: 63. The full-length virus sequence of MVK-014-212-B.1.351 (containing the mKate marker) is provided at SEQ ID NO: 64, and the full-length virus sequence of MV-014-212-B.1.351 (not containing the mKate marker) is provided at SEQ ID NO: 65. The sequence of a BAC comprising MVK-014-212-B.1.351 (containing the mKate marker) is provided at SEQ ID NO: 66, and the sequence of a BAC comprising MV-014-212-B.1.351 (not containing the mKate marker) is provided at SEQ ID NO: 67.

### In Vitro Characterization of MV-014-212

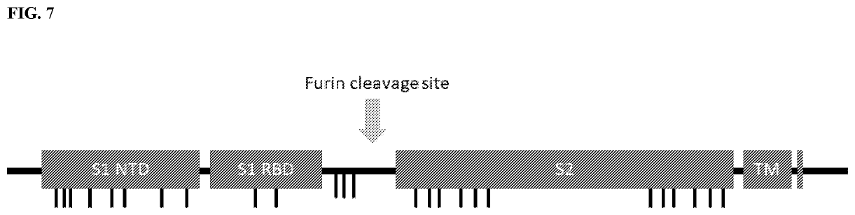

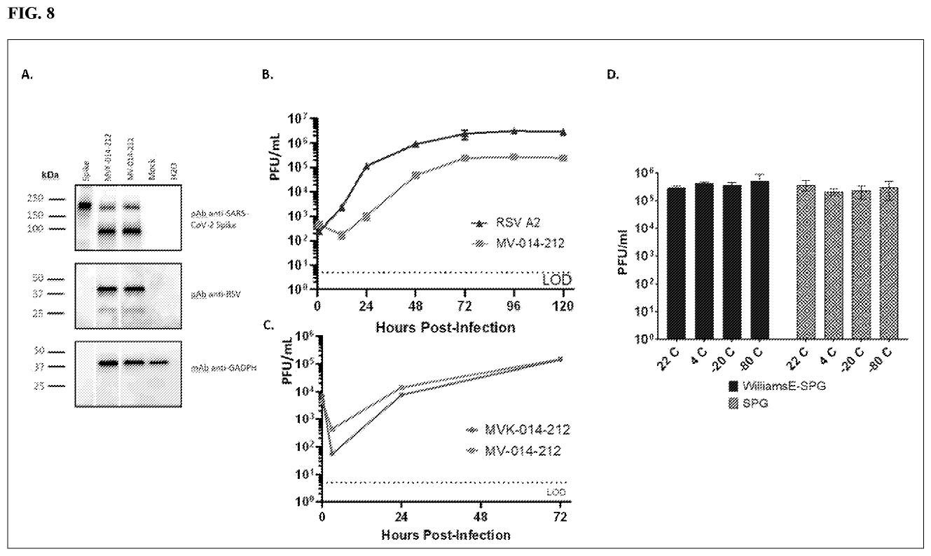

The SARS-CoV-2 spike protein contains a cleavage site between the S1 and S2 domains that is processed by furin-like proteases (FIG. 7 and Hoffmann et al. (2020) Mol Cell 78(4):779-784.e5). As for other coronaviruses, the S1 and S2 subunits of SARS-CoV-2 spike are believed to remain non-covalently bound in the prefusion conformation after cleavage (Walls et al. (2020) Cell 181(2):281-292.e6, Burkard et al. (2014) PLoS Pathog. 10(11):e1004502). To determine if the chimeric spike protein encoded by MV-014-212 is expressed and proteolytically processed, virus stocks prepared from lysates of infected Vero cells were analyzed on western blots and probed with polyclonal antiserum against SARS-CoV-2 spike protein. Both MV-014-212 and MVK-014-212 viruses express the full length and cleaved forms of the chimeric spike protein (FIG. 8A), consistent with partial cleavage at the S1-S2 junction, with apparent sizes in agreement with the expected (FIG. 8A, Ou et al. (2020) Nat Commun. 11(1):1620, Erratum in: Ou et al. (2021) Nat Commun 12(1):2144: Peacock et al. (2020) Nat Microbiol. doi: 10, 1038).

The growth kinetics of MV-014-212 was compared to wild type recombinant RSV A2 in Vero cells (FIG. 8B). Vero cells were infected at an MOI of 0.01 PFU/cell and infectious virus from total cell lysates was quantified by plaque assay at 0, 12, 24, 48, 72, 96 and 120 hours post-infection (hpi). MV-014-212 exhibited delayed growth kinetics relative to RSV A2 showing an initial lag phase of approximately 12 hours. Both viruses reached their peak titers at 72 hpi and the titers remained constant until 120 hpi. The peak titer for MV-014-212 was approximately one order of magnitude lower than that of RSV A2. To determine if the insertion of the mKate2 gene affected replication kinetics of MVK-014-212, Vero cells were infected with MV-014-212 or MVK-014-212 at an MOI of 0.01 PFU/cell and infectious virus was measured at 3, 24 and 72 hpi by plaque assay. The growth kinetics of MVK-014-212 was similar to that of MV-014-212 reaching comparable peak titers by 72 hpi (FIG. 8C). These data are consistent with a report that insertion of mKate2 in the first gene position did not significantly attenuate RSV A2-line19F in vitro (Hotard et al (2012), supra).

To evaluate the short-term thermal stability of MV-014-212, aliquots of the viral stock were incubated at different temperatures for a period of 6 hours and the amount of infectious virus after the incubation was determined by plaque assay. Two stocks of MV-014-212 prepared in different excipients were compared in this study (FIG. 8D). The results demonstrate that MV-014-212 is stable for at least 6 hours in either excipient at −80° C. and room temperature.

The genetic stability of MV-014-212 was examined by serial passaging in Vero cells. Subconfluent Vero cells were infected in triplicate with an aliquot of MV-014-212 and passaged for 10 consecutive passages (FIG. 9). Viral RNA was isolated from passages 0 and 10 and amplified by RT-PCR. The sequence of the entire coding regions of the viral genome was determined by Sanger sequencing. The results showed for all three lineages there were no variation detected at passage 10 relative to the starting stock (passage 0). The vaccine candidate was genetically stable in vitro.

### MV-014-212 Replication is Attenuated in African Green Monkeys and Confers Protection Against Wt SARS-CoV-2 Challenge

African green monkeys (AGMs) support replication of wt SARS-CoV-2 (Woolsey et al. (2021) Nat Immunol. 22(1):86-98, Cross et al. (2020) Virol. 7(1):125, Blair (2021) Am J Pathol. 191(2):274-282, Lee et al. (2021) Curr Opin Virol. 48:73-81) and RSV (Taylor (2017) Vaccine 35(3):469-480) and therefore constitute an appropriate non-human primate model for studying the attenuation and protective immunity of MV-014-212.

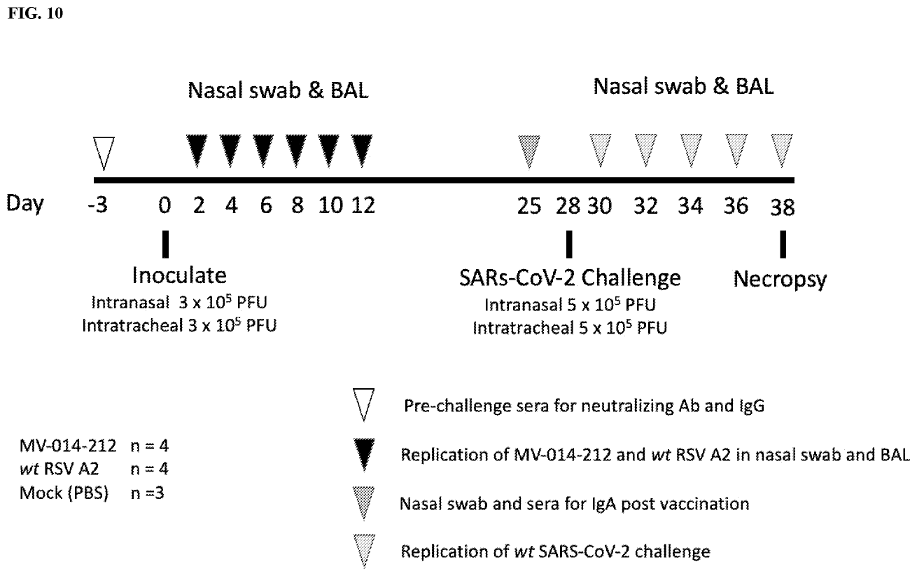

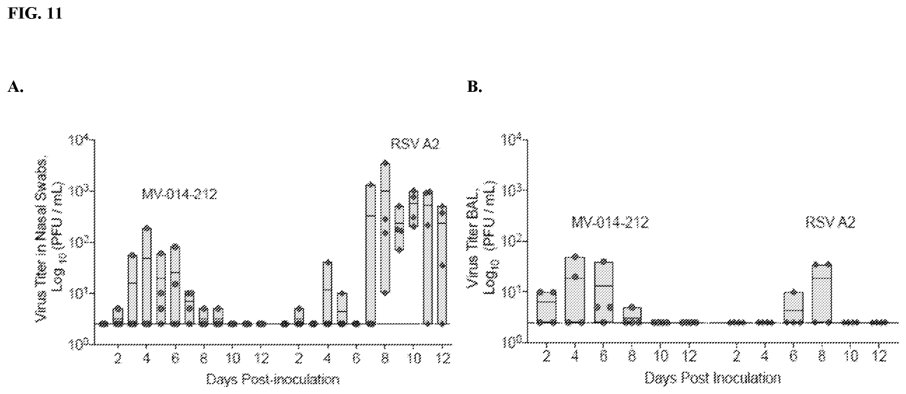

The AGM study design is depicted in FIG. 10. On Day 0, AGMs were inoculated via the intranasal (IN) and intratracheal (IT) routes with 1.0 mL of 3×10⁵ PFU/mL MV-014-212 or wt RSV A2 at each site for a total dose of 6×10⁵ PFU per animal. AGMs are only semi permissive for both SARS-CoV-2 and RSV so intratracheal inoculation was necessary to allow replication of the vaccine or the challenge SARS-CoV-2 in the lungs. Animals in the mock group were similarly mock-inoculated with PBS. Nasal swabs (NS) and Bronchoalveolar lavage (BAL) samples were collected through day 12 after immunization. Viral shedding in NS and BAL samples was determined by plaque assay using fresh samples that were not frozen at the study site. The results, shown in FIGS. 11A-B, showed that the level of infectious virus in animals inoculated with MV-014-212 and duration of shedding in nasal secretions was lower than animals inoculated with RSV (FIG. 11A). The mean peak titer for RSV was approximately 20-fold higher than that observed for MV-014-212 inoculated animals. These results show that MV-014-212 is attenuated in the upper respiratory tract of AGMs compared to RSV.

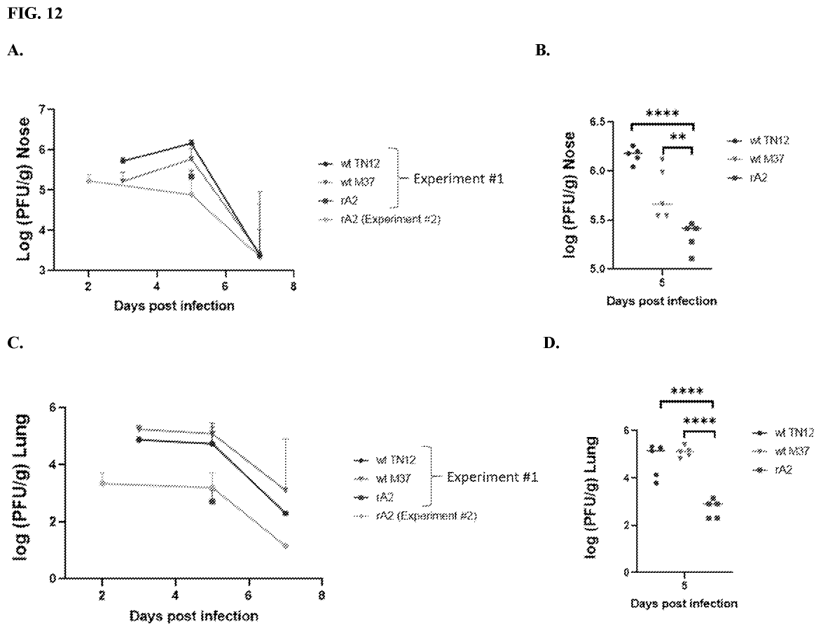

Low to undetectable virus titers were also observed in the lower respiratory tract of animals inoculated with MV-014-212 or RSV strain A2 over the course of 12 days. Both viruses replicated at low levels, but peak levels occurred earlier for MV-014-212. In this study RSV A2 showed 2 to 3 log lower peak titers in the lower respiratory tract of AGMs compared to wild-type RSV A2 titers reported in literature (Cheng et al. (2001) Virology 283(1):59-68; Jin et al. (2003) Vaccine 21(25-26):3647-52; Tang et al. (2004) J Virol. 78(20):11198-207; Le Nouën et al. (2014) Proc Natl Acad Sci USA. 111(36):13169-74) confounding the ability to demonstrate attenuation of MV-014-212 in the lungs. Subsequently, lower rA2 titers were also observed in the lungs of cotton rats (see Example 4 and FIG. 12A-D), relative to biologically derived RSV strains suggesting that rA2 used in this study was attenuated in lungs.

Nasal and BAL samples from day 6 post vaccination were used to extract RNA for sequence analyses of the spike gene of MV-014-212. Using Sanger sequencing no variations in the Spike gene were detected compared to the reference sequence for MV-014-212.

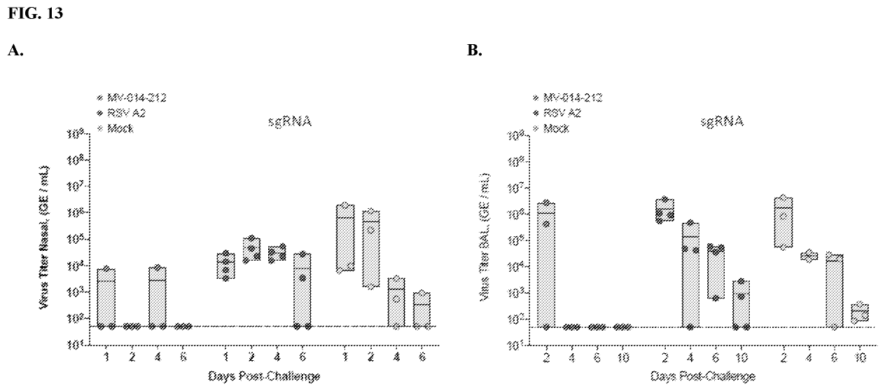

On day 28 AGMs were challenged with 1×10⁶ TCID₅₀ of wt SARS-CoV-2. NS and BAL samples were collected for 10 days after challenge. Shedding of wt SARS-CoV-2 was measured by RT-qPCR of the E gene sub-genomic SARS-CoV-2 RNA (sgRNA) (FIGS. 13A-B).

MV-014-212 vaccinated monkeys had low or undetectable levels of wt SARS-CoV-2 sgRNA in NS samples in contrast to animals inoculated with wt RSV A2 or PBS (mock) which had higher levels of SARS-CoV-2 sgRNA. While the level of SARS-CoV-2 sgRNA was undetectable in animals vaccinated with MV-014-212 at most time points, one animal had detectable SARS-CoV-2 sgRNA at day 2 and a different animal had similar titers at day 4 post-challenge. Mean peak titers of SARS-CoV-2 in NS of animals in the control RSV and PBS groups were 20 and 250-fold higher than for animals vaccinated with MV-014-212, respectively. In both RSV and mock infected animals shedding of wt SARS-CoV-2 sgRNA decreased steadily in nasal secretions from day 4 to 10 and by day 10 all animals in both groups had undetectable SARS-CoV-2 sgRNA.

Vaccination with MV-014-212 increased clearance of SARS-CoV-2 in lungs compared to animals inoculated with RSV A2 or mock-inoculated with PBS. The peak titer of SARS-CoV-2 in BAL samples occurred at Day 2 and was similar in all three treatment groups. Lung titers were undetectable in MV-014-212 vaccinated animals on Day 4 through day 10 while SARS-CoV-2 was readily measured in animals inoculated with RSV A2 or mock-inoculated with PBS. Previously, minor amounts of sgRNA were detected in the inoculum (BIOQUAL, unpublished results) so some of the signal detected on day 1 of shedding could be attributed to the inoculum.

Taken together, these data show that a single mucosal administration of MV-014-212 protected AGMs from wt SARS-CoV-2 challenge.

MV-014-212 Elicits Spike-Specific Antibody Responses in AGMs that are Broadly Neutralizing and Offer Moderate Protection Against a Variant of Concern.

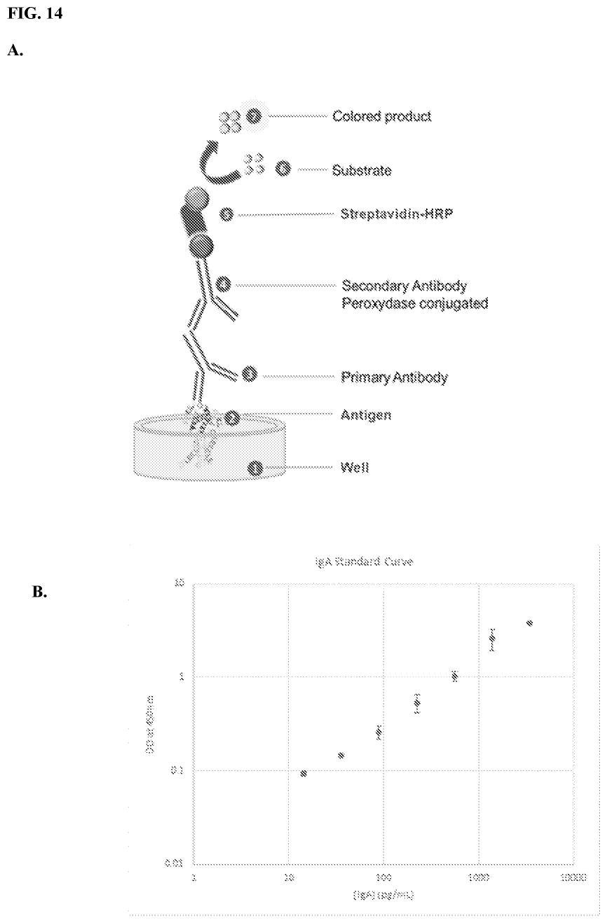

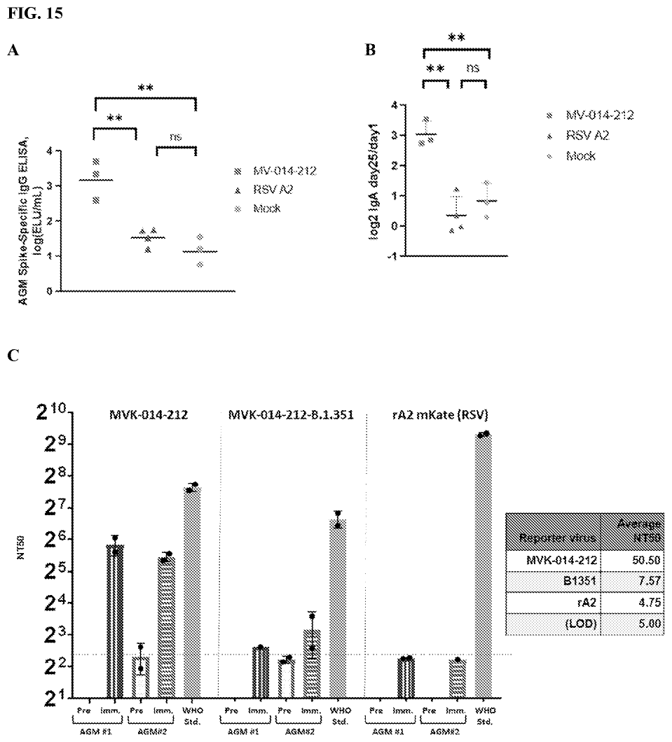

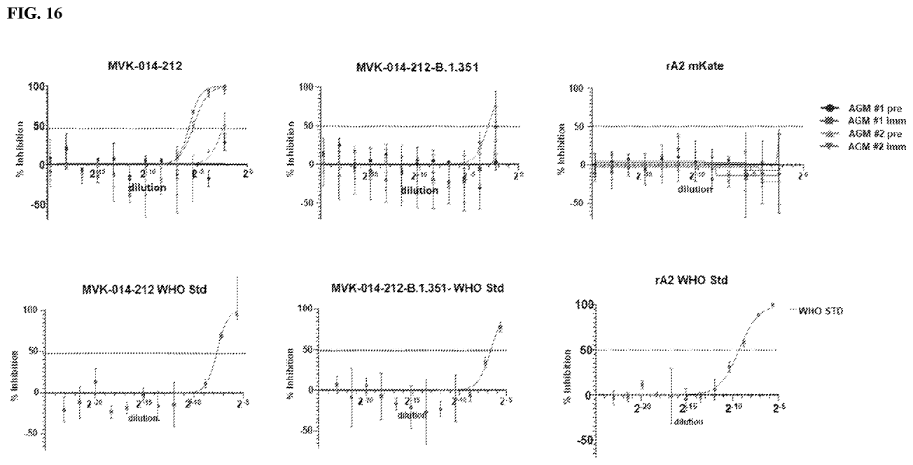

SARS-CoV-2 spike-specific serum IgG and nasal IgA were measured by ELISA (see schematic at FIG. 14A and IgA standard curve at FIG. 14B) in sera and nasal swabs, respectively, from AGMs immunized with MV-014-212, RSV A2, or PBS on Day 25 post-immunization. All animals were seronegative for RSV and SARS-CoV-2 at the start of the study. AGMs inoculated with MV-014-212 produced higher levels of SARS-CoV-2 spike-specific IgG in serum compared to AGMs inoculated with RSV A2 or PBS, which had levels of Spike-specific IgG that were close to the limit of detection (FIG. 15A).

Spike-specific IgA was also detected in the nasal swabs of monkeys inoculated with MV-014-212. There was more than an 8-fold increase in nasal Spike-specific IgA in the MV-014-212 vaccinated animals 25 days after vaccination (FIG. 15B). In contrast, RSV or mock vaccinated animals did not show a significant change in IgA.

These results showed that mucosal inoculation of MV-014-212 induced both nasal and systemic antibody responses to the functional SARS-CoV-2 spike.

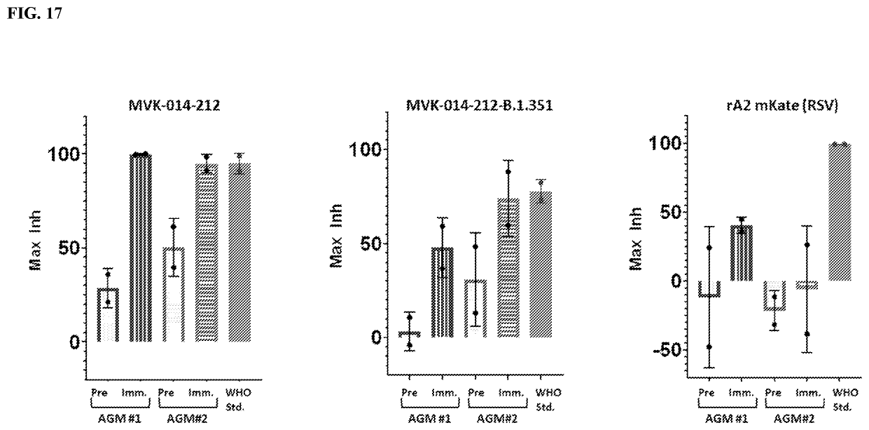

To determine if neutralizing antibodies to wild-type SARS-CoV-2 spike protein or the B.1.351 variant were elicited in monkeys vaccinated with MV-014-212, a microneutralization assay was conducted using the reporter viruses MVK-014-212 and MVK-014-212-B.1.35. An additional reporter virus, wild-type recombinant RSV A2 labelled with mKate2 (rA2-mKate) was included as a negative control. The neutralizing titers for 2 AGMs before (“Pre”) and after vaccination (“Imm”) are shown in FIG. 15C. A significant increase in neutralization was observed for the homologous reporter (MVK-014-212) following vaccination (see also FIG. 17). A moderate cross neutralization against the B.1.351 variant was also observed, with the average NT50 of the variant being approximately 7-fold lower than for the homologous virus. This reduction in neutralizing titers for the B.1.351 variant is of the same order as that reported for other vaccines (Planas et al (2021) Nat Med. 27(5):917-924, Liu et al. (2021), supra, Wang el al. (2021) Nature 592(7855):616-622).

Accordingly, the example demonstrates that infection with MV-014-212 induced a SARS-CoV-2 Spike-specific mucosal IgA response, generated serum neutralizing antibodies against Spike-expressing pseudovirus, including the variant B.1.351, and was highly protective against SARS-CoV-2 challenge in the upper and lower respiratory tract.

### Discussion

MV-014-212 is a recombinant live attenuated COVID-19 vaccine designed to be administered intranasally to stimulate mucosal as well as systemic immunity against SARS-CoV-2. MV-014-212 was engineered to express a functional SARS-CoV-2 spike protein in place of the RSV membrane surface proteins F, G and SH in an attenuated RSV strain expressing codon deoptimized NS1 and NS2 genes. Indeed, replication of MV-014-212 was attenuated in the respiratory tract of African green monkeys following mucosal administrations in the nose and trachea and it elicited S ARS-CV-2 spike-specific mucosal IgA and serum IgG. Furthermore, vaccination with MV-014-212 induced serum neutralizing antibodies and protected against SARS-CoV-2 challenge. These data suggest that a single mucosal immunization with a live attenuated COVID-19 vaccine can induce protective immunity against SARS-CoV-2 in non-human primates.

MV-014-212 is genetically stable and accumulation of variants was not detected when the virus was serially passaged ten times in Vero cells. This contrasts with another recombinant live attenuated COVID-19 vaccine based on the VSV backbone (Yahalorm-Ronen et al. (2020) Nat Commun. 11(1):6402) where mutations arose at passage 9 in Vero E6 cells. One of these mutations occurred in the multi-basic S1/S2 furin cleavage site and another generated a stop codon that resulted in a 24-amino acid truncation of the Spike cytoplasmic tail. Truncation of the Spike cytoplasmic tail was also reported when wt SARS-CoV-2 (Ou, supra) or pseudotyped SARS-CoV -2 (Case et al (2020) Cell Host Microbe 28(3):475-485.e5, Dieterle et al (2020) Cell Host Microbe 28(3):486-496.e6) were propagated in tissue culture. The Spike gene of MV-014-212 from nasal swabs and BAL of African green monkeys was also analyzed by Sanger sequencing and no variations were observed compared to the reference sequence. Therefore, the chimeric Spike gene in MV-014-212 appears to have a stable genotype in vitro and in viva.

African green monkeys are semi permissive for RSV (Taylor, supra) and wt SARS-CoV-2 replication (Woolsey et al, supra, Cross et al., supra, Blair et al, supra, Lee et al. supra) and were selected for evaluating MV-014-212 instead of Rhesus monkeys. MV-014-212 vaccinated monkeys had low or undetectable levels of wt SARS-CoV-2 sgRNA in NS samples after challenge in contrast to RSV and PBS immunized groups. Vaccination with MV-014-212 also increased clearance of SARS-CoV-2 in lungs. wt SARS-CoV-2 shedding detected by RT-qPCR of subgenomic E gene peaked early on day 1 or 2 in the upper and lower respiratory tract of RSV and PBS immunized groups. This was similar to the shedding kinetics detected by RT-qPCR of the viral genome and by plaque assay reported by Cross et al., supra and Woolsey et al., supra for wt SARS-CoV-2/INMI1-Isolate/2020/Italy in AGMs. The levels of peak SARS-CoV-2 subgenomic RNA observed in RSV and mock vaccinated groups were comparable to those observed in unvaccinated Rhesus monkeys (Corbett et al (2020) Nature 586(7830):567-571, Vogel et al (2020) bioRxiv 2020 (09.08.280818; doi: doi.org/10.1101/2020.09.08.280818), Mercado et al (2020) Nature 586(7830):583-588, van Doremalen et al. (2020) Nature 586(7830):578-582).

Immunization of AGM with MV-014-212 resulted in both mucosal and systemic antibody responses. There was approximately 100-fold more Spike-specific total serum IgG in MV-014-212 vaccinated AGM compared to AGM that received wt RSV A2 or PBS inoculations. Spike-specific IgA was also detected in nasal swabs of MV-014-212 immunized animals. There was approximately an 8-fold increase in IgA concentration 25 days following vaccination with MV-014-212. In contrast, RSV or mock-immunized monkeys did not show a rise in IgA concentration. In an experimental human challenge study, low RSV F specific mucosal IgA was a better predictor for susceptibility to RSV challenge in seropositive adults than serum antibody levels (Habibi et al (2015) Am J Respir Crit Care Med. 191(9):1040-9). Indeed, Spike RBD-specific dimeric serum IgA was shown to be more potent at neutralizing SARS-CoV-2 than monomeric IgG (Wang et al, supra). By inference, secretory IgA which exists at mucosal surfaces as dimeric IgA may act as a potent inhibitor of SARS-CoV-2 at the site of infection. Interestingly, Sterlin et al ((2021) Sci Transl Med. 13(577):eabd2223) recently reported that IgA antibodies dominate early humoral responses in human SARS-CoV-2 infections and IgA plasmablasts with mucosal homing potential peaked during the third week of disease onset. A rise in SARS-CoV-2 neutralizing antibody response was detected against MVK-014-212, an mKate2-expressing MV-014-212 virus. A neutralizing antibody response was also detected against a reporter virus with the spike of B.1.351, a variant of concern from South Africa. The NT50 against B.1.351 was approximately 7 fold lower compared to the homologous USA-WA2020 spike. AGMs are semi-permissive for RSV and SARS-CoV-2 precluding a direct comparison to titers observed in human convalescent and post-vaccination serum associated with protection against COVID-19. No correlates of protection have been established in humans for COVID-19 vaccines approved for emergency use. However, MV-014-212 vaccinated AGMs achieved a level of protection that is comparable to those observed with EUA vaccines in Rhesus monkeys (Corbett et al. (2020), supra, Vogel et al. (2021), supra, Mercado et al (2020), supra., van Doremalen et al. (2020), supra).

According to the May 14, 2020 “The Landscape of candidate vaccines in clinical development” (see website at who.int/publications/m/item/draft-landscape-of-covid-19-candidate-vaccines) prepared by WHO, there are 101 COVID-19 vaccines currently in clinical development worldwide. Among these candidates only 7 are intranasal vaccines (TABLE 5). Two other intranasal vaccine candidates are live attenuated viruses. Unlike these vaccine candidates, MV-014-212 is a non-segmented negative strand RNA virus not prone to recombine in nature. RNA recombination is extremely rare for non-segmented negative strand RNA viruses outside of experimental co-infections in laboratory settings and there is no mechanism for reassortmnent (Spaan 2003, Han 2011, Tan 2012).

TABLE 5
Intranasal COVID-19 vaccine in clinical development 2021

| | | | | Phase of |
|---|---|---|---|---|
| | | | | clinical |
| Platform | Description | Doses | Developer | development |
| Live | Attenuated RSV expressing | 1 | Meissa | 1 |
| attenuated | functional Spike protein | | | |
| virus | | | | |
| Live | COVI-VAC | 1-2 | Codagenix/Serum | 1 |
| attenuated | | | Institute of India | |
| virus | | | | |
| Replicating | DelNS1-2019-nCoV-RBD- | 2 | University of Hong | 2 |
| viral vector | OPT1 (Intranasal flu-based- | | Kong, Xiamen | |
| | RBD) | | University and | |
| | | | Beijing Wantai | |
| | | | Biological | |
| | | | Pharmacy | |
| Non | AdCOVID, Adenovirus- | 1-2 | Altimmune, Inc. | 1 |
| replicating | based platform expresses the | | | |
| viral vector | receptor-binding domain | | | |
| | (RBD) of the Sars-Cov-2 | | | |
| | spike protein | | | |
| Non | BBV154, Adenoviral vector | 1 | Bharat Biotech | 1 |
| replicating | COVID-19 vaccine | | International | |
| viral vector | | | Limited | |
| Inactivated | Live recombinant Newcastle | 2 | Laboratorio Avi- | 1 |
| virus | Disease Virus (rNDV) vector | | Mex | |
| | vaccine | | | |
| Protein | CIGB-669 (RBD + AgnHB) | 3 | Center for Genetic | 1/2 |
| subunit | | | Engineering and | |
| | | | Biotechnology | |
| | | | (CIGB) | |

The vaccine profile of MV-014-212 is unique among the current COVID-19 vaccines that have emergency use authorization or are in clinical development. MV-014-212 is administered intranasally, a needle-free route that offers potential advantages for global immunization. The intranasal route is similar to the natural route of infection of SARS-CoV-2 and generates both mucosal and humoral immune responses in AGMs without any adjuvant formulation. Modeling based on yields from production of Phase 1 clinical study material projected a potential close output of hundreds of millions of doses per annum in a modestly sized facility using high intensity bioreactor systems. Mucosally delivered live attenuated vaccines such as MV-014-212 entails minimum downstream processing and has an anticipated low cost of goods. In addition, needle-free delivery reduces supply risks. Overall, MV-014-212 is well-suited for domestic and global deployment as a primary vaccine or as a heterologous booster. M4V-014-21 I's currently being evaluated as a single dose intranasal vaccine in a Phase I clinical trial (NCT04798001).

### Materials and Methods

### Cells and Animals

Vero RCB1 (WHO Vero RCB 10-87) cells were grown in minimal essential medium (MEM, Gibco) containing 10% fetal bovine serum (FBS, Corning) and 1× Corning Antibiotic/Antimycotic mix consisting of 100 I.U./mL Penicillin, 100 μg/mL Streptomycin 0.25 μg/mL Amphotericin with 0.085 g/L NaCI. RCB2 Cells are derived from RCB1 and have been adapted to grow in serum-free media. RCB2 cells used in this study were grown in serum-free medium OptiPro (Gibco) supplemented with 4 mM of L-glutamine (Gibco). Both Vero cell lines were cultured at 37° C., 5% CO₂ with 95% humidity.

African green monkeys (Chlorocebus aethiops) were obtained in St Kitts and were of indeterminate age, weighing ˜3-6 kg. The monkeys were screened and verified to be seronegative for RSV and SARS-CoV-2 by an RSV microneutralization assay and spike SARS-CoV-2 ELISA (BIOQUAL). Animals also underwent a physical examination by the veterinary staff to confirm appropriate health status prior to study. Each AGM was uniquely identified by a tattoo. One male and 3 females were assigned to the MRV-014-212 and RSV groups. 2 females and 1 male were assigned to the Mock group. Cage-side observations included mortality, moribundity, general health and signs of toxicity. Clinical observations included skin and fur characteristics, eye and mucous membranes, respiratory, circulatory, autonomic and central nervous systems, somatomotor, and behavior patterns. The body weight of each monkey was recorded before the start of the dosing period and at each time of sedation. Consistent with the overall low levels of MV-014-212 replication in the respiratory tract of AGMs, no adverse events that were considered treatment-related were observed following inoculation with the vaccine. On Day 16 post-vaccination one monkey inoculated with MV-014-212 died unexpectedly. Death occurred 4 days after the last NS and BAL sample collection. A definitive determination of the cause of death could not be ascertained based on macroscopic or microscopic postmortem evaluations; however, there was no evidence that suggests the death was vaccine related. Moreover, the deceased animal had the lowest titer in NS samples compared to the other animals in this treatment group with only one swab containing virus that was above the detection limit of the plaque assay (50 PFU/mL) and no detectable infectious virus in BAL at any of the time points evaluated.

Male and female K18-hACE2 Tg (strain #034860, B6.Cg-Tg[K18-ACE2]2Prlmn/J) mice were procured from The Jackson Laboratory (Bar Harbor, Me.) and were approximately 8-10 weeks old at the time of vaccination.

The animal studies were conducted in compliance with all relevant local, state, and federal regulations and were approved by the BIOQUAL Institutional Animal Care and Use Committee (IACUC).

### Plasmid Construction

The recombinant MV-014-212 and derived viruses were cloned in the antigenome orientation in bacterial artificial chromosomes (BAC) under the control of the T7 polymerase promoter (Hotard et al (2012), supra). The BACs containing the recombinant MV-014-212 and MVK-014-212 sequences were constructed by restriction digestion and ligation from the DB1-QUAD and kRSV-DB1-QUAD plasmids (encoding the antigenome of an attenuated version of RSV with or without the mKate gene, respectively, Rostad el al. (2018), supra). The DNA sequence encoding the chimeric spike protein was designed to contain compatible cloning sites and it was synthesized by Twist Biosciences. The kRSV-DB1-QUAD plasmid and spike insert were digested with the enzymes AatII and SalI (NIB) and ligated with T4 DNA ligase (NIB) overnight at 16° C. Stabl3 chemically competent cells (Invitrogen) were transformed with the ligation mix and selected for chloramphenicol resistance for 20-24 hours at 32° C. MV-014-212 BAC was derived from the MVK-014-212 vector by removing the fragment between the KpnI and AatII restriction sites (˜7 kb containing the mKate gene) and replacing it with the corresponding fragment extracted from DB1-QUAD by restriction digestion and ligation. For all the constructs, the sequences of the entire encoded viruses were confirmed via Sanger sequencing.

The construction of the plasmid rA2-mkate (a.k.a. kRSV-A2) was described in Rostad el al. (2016) J Virol. 90(16):7508-7518.

### Virus Rescue and Harvest

The recombinant viruses were rescued by electroporation of RCB2 cells with the BAC plasmid and 5 helper plasmids based on the pCDNA3.1 expression plasmid, each encoding one of the following: T7 polymerase, RSV A2 N, RSV A2 P, RSV A2 M2-1 or RSV A2 L proteins. The cells were recovered in SFM-OptiPro medium supplemented with 4 mM glutamine and 10% fetal bovine serum (Hyclone) for 2 passages and then expanded in serum free medium with glutamine until CPE was extensive.

The recombinant viruses were harvested in Williams E (Hyclone) supplemented with SPG or SPG alone by scraping the infected cells directly into the media. The lysate was vigorously vortexed to release the viral particles and flash frozen. One cycle of thawing and vortexing was performed to increase the release of virus before the stocks were aliquoted, flash-frozen and stored at −70° C. until use.

The composition of the SPG medium is shown in TABLE 6.

TABLE 6
Composition of SPG

| | | | | | Final |
|---|---|---|---|---|---|
| | | Catalog | | Quantity | Molarity |
| Ingredient | Supplier | number | Lot number | (g) | (M) |
| Potassium phosphate | JT | 3250 | 0000246987 | 13.56 | 0.078 |
| Dibasic (K₂HPO₄) | Baker/Avantor | | | | |
| Potassium phosphate | JT | 3248 | 0000248923 | 5.17 | 0.038 |
| Monobasic (KH₂PO₄) | Baker/Avantor | | | | |
| Sucrose C₁₂H₂₂0₁₁ | JT | 4074 | 0000243012 | 746.22 | 2.18 |
| | Baker/Avantor | | | | |
| L-Glutamic acid | Sigma Aldrich | G8415 | SLCC1249 | 7.94 | 0.054 |
| HO₂CCH₂CH₂CH(NH₂)CO₂H | | | | | |
| 5N Sodium Hydroxide | EMD | SX0607L-6 | HC97338720 | TBD | Trace |
| (amount to adjust to pH | Millipore | | | | |
| 7.1) | | | | | |
| WFI Water | HyClone | SH30221.10 | AE29421224 | Adjust to | NA |
| | | | | one liter | |

Plaque assays for all the viruses used were done in 24-well plates with Vero cells. Cells at 70% confluence were inoculated with 100 μl of 10-fold serial dilutions of viral samples (10-1 to 10-6). Inoculation was carried out at room temperature with gentle rocking for 1 h before adding 0.75% methylcellulose (Sigma) dissolved in MEM supplemented with 10% FBS and IX Corning Antibiotic/Antimycotic mix. Cells were incubated for 4-5 days at 32° C. before fixing in methanol and immunostaining. For MV-014-212 and MVK-014-212 we used Rabbit anti-SARS-CoV-2 spike polyclonal antibody (Sino Biological) and Goat anti-rabbit HRP-conjugated secondary antibody (Jackson ImmunoResearch). For rA2-mKate the reagents used were Goat anti-RSV primary antibody (Millipore) and Donkey anti-goat HRP-conjugated secondary antibody (Jackson ImmunoResearch). In all cases, the viral plaques were stained with AEC (Sigma). The limit of detection is 1 PFU per well, corresponding to a minimum detectable titer of 100 PFU/ml.

### RNA Sequencing

RNA from MV-014-212 samples was extracted using QIAamp® Viral RNA Mini Kit following the protocol suggested by the manufacturer. The quality and concentration of the extracted RNA were evaluated by gel electrophoresis and UV spectrophotometry. The extracted RNA was used as the template for reverse transcription (RT) using Invitrogen SuperScript® IV First-Strand Synthesis System using a specific primer or random hexamers. The cDNA 2nd strand was synthesized with the Platinum™ SuperFi™ PCR Master Mix. The purified PCR products were directly sequenced using the BigDye® Terminator v3.1 Cycle Sequencing Kit (Applied Biosystems). The sequencing reactions were purified using Sephadex G-50 purification and analyzed on ABI 3730xl DNA Analyzer. The sequence traces were assembled using Sequencher software and the assembly was manually confirmed. The RNA sequencing for this study was performed by Avance Biosciences Inc., Houston Tex.,

### Western Blot

Viruses and control recombinant SARS-CoV-2 Spike protein (LakePharma, San Carlos, Calif.) were denatured with Laemmli sample buffer (Alfa Aesar, Ward Hill, Mass.) by heating at 95° C. for 10 minutes. Proteins were separated by SDS-PAGE in a 4-15% gradient gel and transferred to PVDF membranes using a transfer apparatus according to the manufacturer's protocol (BIO-RAD, Hercules, Calif.). After transfer, blots were washed in deionized water and probed using the iBind Flex system according to the manufacturer's protocol. Rabbit anti-SARS-CoV-2 Spike (Sino Biological Inc, Beijing, China) was diluted in iBind solution (Invitrogen, Carlsbad, Calif.) at 1:1000. HRP conjugated anti-Rabbit IgG (Jackson ImmunoResearch, Philadelphia, Pa.) was diluted in iBind Solution at 1:5000. Blots were washed in deionized water and developed with ECL system (Azure Biosystems, Dublin, Calif.) according to manufacturer's protocol. The blots were stripped with Restore Western Blot Stripping Buffer (ThermoFisher, Carlsbad, Calif.) and reprobed with goat anti-RSV polyclonal antisera (Sigma-Aldrich, St. Louis, Mo.) and a monoclonal antibody specific for GAPDH (6C5) protein (ThermoFisher, Carlsbad, Calif.).

### Plague Assay for Detecting Virus Shedding in AGMs

Nasal swabs (NS) and bronchoalveolar lavage (BAL) samples were collected and stored on ice until assayed for vaccine shedding by plaque assay. Vero cells were seeded in 0.5 mL per well at 1×10⁵ cells/mL in culture media in 24 well plates. The plates were incubated overnight at 37° C. in a humid incubator containing 5% CO₂. The samples were diluted in DMEM without serum by adding 30 μL of nasal swab or BAL to 270 μL of DMEM. A total of six 10-fold serial dilutions were prepared in DMEM from 10-1 to 10-6. The media was removed from the 24 well plate and 100 μL of each dilution was added to duplicate wells of the 24 well plate of Vero cells. The plate was incubated at room temperature with constant rocking on a Rocker 35EZ, Model Rocker 35D (Labnet, Edison, N.J.) for 1 h. At the end of this incubation, 1 mL of methyl cellulose media (MEM supplemented with 10% fetal bovine serum, 1× antibiotic/antimycotic, and 0.75% methyl cellulose) was added to each well. The plate was incubated at 34° C. for 6 days in a humid incubator containing 5% CO₂.

The plaques were visualized by immunostaining using RSV or SARS-CoV-2 antibodies. For immunostaining, the methyl cellulose media was aspirated, and the cell monolayers were washed with 1 mL of PBS at room temperature. The PBS was removed, and the cells were fixed by the addition of 1 mL of methanol to each well and the plate was incubated at room temperature for 15 minutes. The methanol was removed and cells washed with 1 mL of PBS followed by the addition of 1 mL Blotto solution (5% non-fat dried milk in Tris-buffered saline, Thermo-Fisher). The plates were incubated at room temperature for 1 h. The Blotto solution was removed, and 0.25 mL of primary goat anti-RSV polyclonal antibodies (Millipore, Hayward, Calif.) diluted 1 to 500 in Blotto was added to RSV infected cells. Cells infected with MV-014-212 were stained with primary rabbit anti-SARS-CoV-2 spike protein polyclonal antisera (Sino Biologicals, Beijing, CN). The plates were incubated for 1 h at room temperature with constant rocking. Primary antibodies were removed and wells were washed with 1 mL Blotto solution.

For RSV infected cells, 0.25 mL of donkey anti-goat HRP-conjugated polyclonal antisera (Jackson ImmunoResearch, West Grove, Pa.) diluted 1:250 in Blotto was added to each well. For MV-014-212 infected cells goat anti-rabbit HRP-conjugated polyclonal antisera (Jackson ImmunoResearch, West Grove, Pa.) diluted 1:250 in Blotto was added to each well. The pate was incubated for 1 h at room temperature with constant rocking. After incubation, the secondary antibodies were removed and the wells washed with 1 mL of PBS. Developing solution was prepared by diluting AEC substrate 1 to 50 in 1×AEC buffer solution. A total of 0.25 mL of developing solution was added to each well and the plate was incubated at room temperature for 15 to 30 minutes with constant rocking until red immunostained plaques were visible by eye. The developing reaction was terminated by rinsing the plate under tap water. The plaques were enumerated, and titers were calculated.

### RT-qPCR of SARS-CoV-2 Sub Genomic RNA for Detecting Shedding of Challenge Virus

The standard curve was prepared from frozen RNA stocks and diluted to contain 106 to 107 copies per 3 μL. Eight 10-fold serial dilutions of control RNA were prepared using RNAse-free water to produce RNA concentrations ranging from 1 to 107 copies/reaction.

The plate was placed in an Applied Biosystems 7500 Sequence detector and amplified using the following program: 48° C. for 30 minutes, 95° C. for 10 minutes followed by 40 cycles of 95° C. for 15 seconds, and 1 minute at 55° C. The number of copies of RNA per mL of sample was calculated based upon the standard curve.

Total RNA from tissues was extracted using RNA-STAT 60 (Tel-test “B”)/chloroform followed by precipitation of the RNA and resuspension in RNAse-free water. To detect SARS-CoV-2 sgRNA, a primer set and probe were designed to detect a region of the leader sequence and E gene RNA from SARS-CoV-2. The F gene mRNA is processed during replication to contain a 5′ leader sequence that is unique to sgRNA (not packaged into the virion) and therefore can be used to quantify sgRNA. A standard curve was prepared using known quantities of plasmid DNA containing the E gene sequence including the unique leader sequence to produce a concentration range from 1 to 10⁶ copies/reaction. The PCR reactions were assembled using 45 μL master mix (Bioline, Memphis, Tenn.) containing 2× buffer, Taq-polymerase, reverse transcriptase and RNAse inhibitor. The primer pair was added at 2 μM. 5 μL of the sample RNA was added to each reaction in 96-well plate. The PCR reactions were amplified in an Applied Biosystems 7500 Sequence detector using the following conditions: 48° C. for 30 minutes, 95° C. for 10 minutes followed by 40 cycles of 95° C. for 15 seconds, and 1 minute at 55° C.

Primers/Probe sequences are shown below:

SG-F:
(SEQ ID NO: 127)
CGATCTTGTAGATCTGTTCCTCAAACGAAC
SG-R:
(SEQ ID NO: 128)
ATATTGCAGCAGTACGCACACACA
(SEQ ID NO: 129)
FAM-ACACTAGCCATCCTTACTGCGCTTCG-BHQ

### SARS-CoV-2 Total IgG ELISA for AGM Sera

MaxiSorp immuno plates (Thermo-Fisher, Waltham, Mass.) were incubated overnight at 4° C. with 100 μL of 0.65 μg/mL of SARS-CoV-2 spike prepared in PBS (Pre-S SARS-CoV-2 Spike, Nexelis). The protein solution was removed and the plate was washed 4 times with 250 μL of PBS supplemented with 0.05% Tween 20 (PBST). Blocking solution (PBST containing 5% non-fat dried milk) was added at 200 μL per well and the plate was incubated for 1 h at room temperature. A SARS-CoV-2 spike specific IgG (Nexelis) was diluted in blocking solution and used as a standard. Negative control serum was diluted 1:25 in blocking solution. Serum samples were diluted at 1:25 followed by eight 2-fold serial dilutions in blocking solution. The blocking solution was removed from the plate and the wells washed once with 250 μL of PBST followed by addition of 100 μL of the diluted serum samples and controls and the plate was incubated for 1 h at room temperature. The plate was washed 4 times with 250 μL PBST and 100 μL of HRP-conjugated goat anti-monkey IgG antibody (PA1-8463, Thermo Fisher, Waltham, Mass.) diluted in blocking solution was added to each well following the last wash step. The plate was incubated for 1 h at room temperature and then washed 4 times in 250 μL PBST. Developing solution containing 3, 3′, 5, 5′-Tetramethylbenzidine (TMB) substrate (1-Step Ultra TMB-ELISA Substrate Solution, ThermoFisher) was added to each well and the plate was incubated at room temperature for 30 minutes to allow the color to develop. The colorimetric reaction was terminated by the addition of 100 μL of ELISA Stop Solution (Invitrogen). The absorbance at 450 nm and 650 nm was read by spectrophotometry using a SpectraMax iD3 microplate reader (Molecular Devices, San Jose, Calif.).

### SARS-CoV-2 IgA ELISA for AGM Nasal Swabs

Purified pre-fusion SARS-CoV-2 spike antigen (LakePharma) was adsorbed onto 96-well MaxiSorp immuno microplate (Thermo-Fisher). The positive control was a serum pool from three COVID-19 convalescent individuals (Nexelis). Total IgA purified from human serum was used as a standard (Sigma-Aldrich, St. Louis, Mo.). To generate the IgA standard curve anti-human IgA capture antibodies Mab MT57 (MabTech) were absorbed on plates instead of Spike antigen. Following incubation, the microplate was washed 4× with 250 μL. PBST and blocked with 1% BSA in PBST. Purified human IgA standard, controls or sample dilutions were then added and incubated in the coated microplate to allow binding. The plates were washed and a biotinylated goat anti-human IgA antibody (Mabtech) with cross reactivity to monkey antibodies was added to all wells. Excess biotinylated anti-IgA antibody was removed by washing and streptavidin-conjugated HRP (Southern Biotech) was added. TMB was added and color development was stopped by addition of stop solution from Invitrogen. The absorbance of each well was measured at 450 nm. The standard total IgA antibody assayed on each test plate was used to calculate the concentration of IgA antibodies against spike protein in the AGM samples expressed in the arbitrary units ELU/mL. The measurements were performed in duplicates and average values are reported with standard deviations.

### Microneutralization Assay

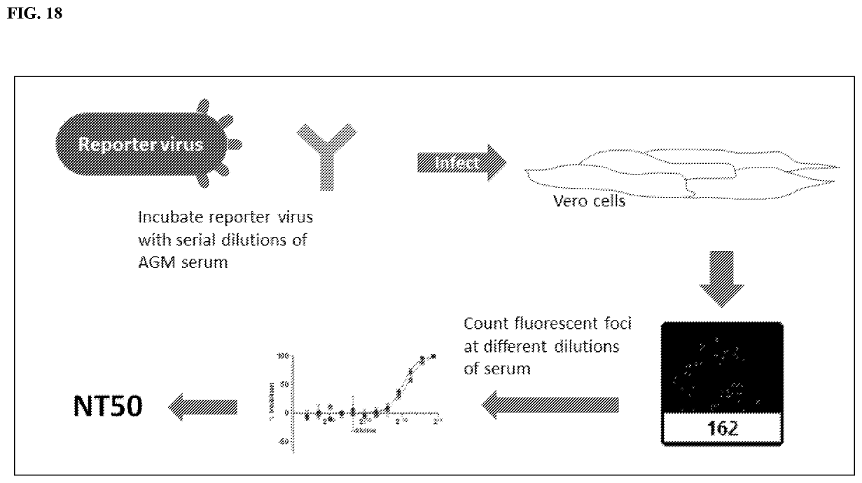

As shown in the schematic in FIG. 18, heat-inactivated sera from the AGMs were diluted serially in MEM with non-essential amino acids (Gibco) and antibiotics/antimycotic. All experiments were done in duplicate. 200 PFU of the desired reporter virus were added to each dilution and incubated at room temperature for one hour. Confluent RCB1 cells grown in a clear-bottom black 96-well plate (Grenier) were infected with the serum-virus mixes and centrifuged (spinoculated) at 1,800×g for 30 minutes at 20° C. The plates were incubated for 20 h at 37° C. and 5% CO₂. The fluorescent foci in each well were counted using a Celigo Image Cytometer (Nexcelom) and converted to % inhibition using the formula below:

$$\% inhibition = 100 \times [ 1 - \frac{( L - MIN )}{( MAX - MIN )} ]$$

where MIN is the average number of foci obtained in the control wells with only cells (no virus) and MAX is the average number of foci from the wells in the control wells with only virus (no serum). L is the number of foci in the sample wells. The resulting curves of inhibition vs. dilution of the sera were fitted using non-linear regression, option “[inhibitor] vs normalized response-variable slope” in GraphPad Prism (version 9.0.0). From the fitting, IC50 was obtained and NT50 was calculated as the reciprocal of IC50.

### Example 4—MV-014-212 Elicits Th1 Skewed Cellular Immune Response in hACE2-Mice

Mice models of vaccine-associated enhanced respiratory disease (VAERD) suggest that an imbalance in type 1 (Th1) and type 2 (Th2) T helper cell immunity with a skewing towards Th2 response contributes to enhanced lung pathology following challenge (Boelen 2000). To assess the balance of Th1 and Th2 immunity generated after vaccination with MV-014-212, transgenic mice expressing human ACE-2 receptor were inoculated with a single dose of V-014-212 or PBS by the intranasal route. A control group received an intramuscular prime and boost vaccination with SARS-CoV-2 spike protein formulated in alum which has been shown to skew immunity towards a Th2 response (Corbett et al., supra). On Day 28, serum was collected to measure total spike-specific IgG, IgG2a and IgG1 by ELISA. In addition, spleens were collected and the number of splenocytes expressing interferon-γ (IFNγ) and IL-5 were measured by ELISpot assay. The ratio of IgG2a/IgG1 and the ratio of cells producing IFNγ/IL-5 are indicators of Th1-biased cellular immune response (Corbet et al., supra, van der Fits et al. (2020) NPJ Vaccines 5(1):49).

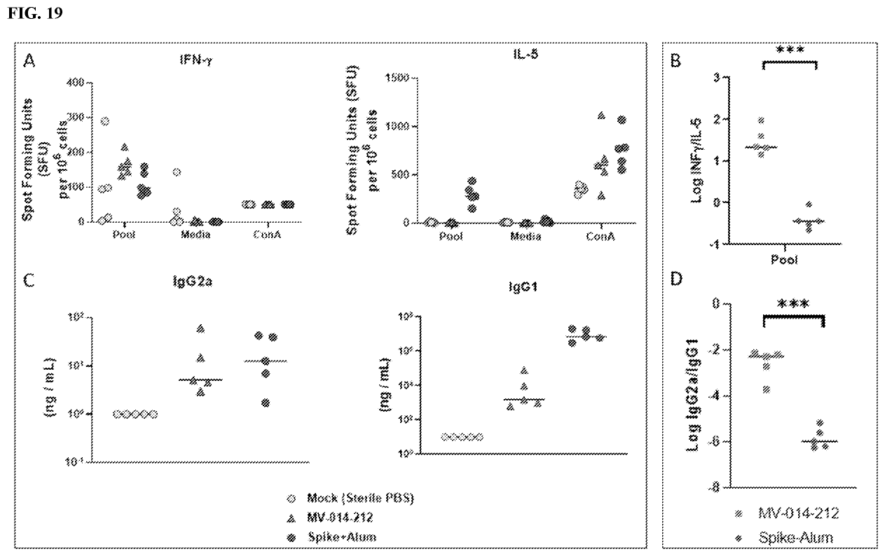

The results showed that MV-014-212 induced spike-reactive splenocytes as measured by ELISpot assay (FIG. 19A). Importantly, MV-014-212 induced higher numbers of splenocytes expressing IFNγ relative to IL-5 when cell suspensions were stimulated with a spike peptide pool suggesting that vaccination with MV-014-212 produced a Th1-biased immune response. The ratio of IFNγ producing cells to IL-5 producing cells in the MV-014-212 group was more than one order of magnitude higher than in the group vaccinated with alum-adjuvanted spike protein, (FIG. 19B). Consistent with the ELISpot data, the ratios of IgG2a/IgG1 detected in serum were higher in the animals vaccinated with MV-014-212 than the control group vaccinated with alum-adjuvanted spike (FIGS. 19C and D). These data suggest that intranasal vaccination with live attenuated, recombinant MV-014-212 induced a Th1-biased antiviral immune response.

### Discussion

In the mice model, MV-014-212 immunization elicited a Th1-biased cellular immune response. More IFNγ producing T cells were detected in splenocytes of hACE mice immunized with MV-014-212 than IL-5 secreting T cells. Furthermore, the ratio of IgG2a/IgG1 isotypes in MV-014-212 hACE2 mice was approximately 1000-fold higher compared to hACE2 mice that received alum-adjuvanted Spike protein immunization. No correlates of protection have been established for COVID-19 vaccines approved for emergency use. However, MV-014-212 vaccinated AGMs achieved a level of protection that is comparable to those observed with EUA vaccines (Corbett et al., supra, Vogel et al., supra, Mercado et al., supra, van Doremalen et al., supra).

### Materials and Methods

SARS-CoV-2 Total IgG ELISA hACE2-Mice

SARS-CoV-2 spike protein was linked to the spike protein signal sequence at the N terminus and a histidine tag was added to the C terminus of the protein. The SARS-CoV-2 spike protein was expressed in HEK293T cells and purified to homogeneity on a AKTA chromatography system using Ni-Sepharose Excel (GE) resin (Global Life Sciences Solutions, Marlborough, Mass.). MaxiSorp immuno plates (Thermo-Fisher, Waltham, Mass.) were incubated overnight at 4° C. with 100 μL of 0.5 mg/mL of SARS-CoV-2 spike prepared in PBS. The protein solution was removed and the plate was washed 3 times with 300 μL of PBS supplemented with 0.1% Tween 20 (PBST). Blocking solution (PBST containing 5% non-fat dried milk) was added at 200 μL per well and the plate was incubated for 1 h at 37° C. A SARS-CoV-2 spike-specific IgG was diluted in blocking solution and used as a standard. Positive and negative control sera was diluted 1:25 in blocking solution. Positive control serum was generated at Nexelis by immunizing mice with the SARS-CoV-2 RBD protein. Negative control serum was obtained from naïve mice. Serum samples were diluted at 1:25 followed by eight 2-fold serial dilutions in blocking solution. The blocking solution was removed from the plate and the wells washed once with 300 μL of PBST followed by addition of 100 μL of the diluted serum samples and controls. The plate was incubated for 2 h at 37° C. Following incubation plate was washed 3 times with 300 μL PBST and 100 μL of HRP-conjugated goat anti-mouse antibody (A140-201P; Bethyl Laboratories, Montgomery, Tex.) diluted in blocking solution was added to each well following the last wash step. The plate was incubated at for 1 h at 37° C. and then washed 3 times in 300 μL PBST. Developing solution containing 3, 3′, 5, 5′-Tetramethylbenzidine (TMB) substrate (BioRad, Hercules, Calif.) was added to each well and the plate was incubated at 37° C. for 30 min to allow the color to develop. The colorimetric reaction was terminated by the addition of 100 μL of 0.36 N sulfuric acid stop solution. The absorbance at 450 nm and 650 nm was read by spectrophotometry using a SpectraMax iD3 microplate reader (Molecular Devices, San Jose, Calif.).

### Spike Specific IgG1 and IgG2a ELISA

Serum samples from mice were collected on Day −21 and on Day 28 post vaccination to quantify the levels of SARS-CoV-2 spike-specific IgG1 and IgG2a antibodies by ELISA. Purified perfusion-stabilized SARS-CoV-2 spike protein (SARS-CoV-2/human/USA/WA1/2020, from LakePharma) was diluted to 1 μg/mL in PBS and 100 μL was added to each well of a Maxisorp immuno plates (Thermo-Fisher) and incubated overnight at 4° C. The plate was washed 4 times in PBST (PBS+0.05% Tween 20) and 100 μL of blocking solution (PBST+2% BSA) was added to each well and the plate was incubated for 1 hour at room temperature. Serum dilutions were prepared in blocking solution with the first dilution at 1:25 for the IgG1 assay or 1:10-1:100 for the IgG2a assay. SARS-CoV-2 spike IgG1 (Sino Biological) or anti-spike-RBD-mIgG2a (InvivoGen) were diluted in blocking solution and used as standards for the assay.

The blocking solution was removed and 100 μL of diluted antibody were added to each well. The plate was incubated at room temperature for 1 h and then washed 4 times in PBST using the plate washer. Then, 100 μL of HRP-conjugated goat-anti-mouse IgG1 (Thermo Fisher) or HRP-conjugated goat-anti-mouse IgG2a (Thermo Fisher) secondary antibodies diluted 1:32,000 and 1:1000, respectively, were added to each well and the plate was incubated at room temperature for 1 h. The plate was washed 4 times in PBST. 100 μL of 1-step ultra TMB-ELISA substrate solution (Thermo Fisher) was added to each well and the plate incubated for 30 min with constant rocking on an orbital shaker. After the incubation period, 100 μL of stop solution (Invitrogen) was added to each well and the plate read on a Spectramax id3 plate reader (Molecular Devices) at 450 nm and 620 nm.

ELISPOT of Splenocytes from MV-014-212 Vaccinated hACE2-Mice

Spleens from vaccinated ACE-2 mice were collected on Day 28 post-inoculation and stored in DMEM containing 10% FBS on ice until processed. The spleens were homogenized on a sterile petri dish containing medium. The homogenate was filtered through a 100 μm cell strainer and the cell suspension transferred to a sterile tube on ice. The cells were collected by centrifugation at 200×g for 8 min at 4° C. The supernatant was removed and residual liquid on the edge of the tube blotted with a clean paper towel. Red blood cells were lysed by resuspending the cell pellet in 2 mL of ACK lysis buffer (155 mM ammonium chloride, 10 mM potassium bicarbonate, 0.1 mM EDTA) and incubating the samples at room temperature for approximately 5 min. PBS was added at 2× to 3× of the volume of cell suspension and cells were collected by centrifugation at 200×g for 8 min at 4° C. The cell pellet was washed twice in PBS and the cells collected by centrifugation at 200×g for 8 min at 4° C. The supernatant was removed and the pellet resuspended in 2 mM L-Glutamine CTL-Test Media (Cell Technology Limited, OH, USA). The suspension was filtered through a 100 μm cell strainer into a new 15 mL conical tube and the cells counted using a hemocytometer and resuspended at the appropriate cell concentration. Cells were maintained at 37° C. in a humidified incubator with 5% CO₂ until used in the ELISpot assay.

The ELISpot assay was performed using a mouse IFNγ/IL-5 Double-Color ELISPOT assay kit (Cell Technology Limited, OH, USA). Murine IFNγ/IL-5 Capture Solution and 70% ethanol was prepared according to the manufacturer's protocol (Cell Technology Limited, OH, USA). The membrane on the plate was activated by addition of 15 μL of 70% ethanol to each well. The plate was incubated for less than one minute at room temperature followed by addition of 150 μL PBS. The underdrain was removed to drain the solution in the wells and each well was washed twice with PBS. Murine IFNγ/IL-5 Capture Solution (80 μL) was added to each well and the plate was sealed with parafilm and incubated at 4° C. overnight. The Capture Solution was removed and the plate washed one time with 150 μL PBS. A peptide pool containing peptides of 15 amino acids in length that span the SARS-CoV-2 spike protein (PepMix™ SARS-CoV-2 Spike Glycoprotein, JPT Peptide Technologies, Berlin Del.) were prepared at 10 mg/mL and 100 μL was added to each well. A positive control containing Concanavalin A (Con A) mitogen (10 μg/mL) was added to a separate reaction mixture. The splenocytes were mixed with CTL-Test™ Medium (Cell Technology Limited, OH, USA) to yield a final cell density of 3,000,000 cells/mL and 100 μL/well were added to the plate using large orifice tips. The plate was incubated at 37° C. in a humidified incubator containing 9% CO2 for 24 hours. The plates were washed twice with PBS and then twice with 0.05% Tween-PBS at a volume of 200 μL/well for each wash followed by addition of 80 μL/well anti-murine IFNγ/IL-5 Detection Solution (Cell Technology Limited, OH, USA). The plates were incubated at room temperature for two hours. The plate was washed three times with PBST at 200 μL/well for each wash followed by the addition of 80 μL/well of Tertiary Solution (Cell Technology Limited, OH, USA). The plates were incubated at room temperature for one hour. The plate was washed twice with PBST, and then twice with 200 μL/well of distilled water. Blue Developer Solution (Cell Technology Limited, OH, USA) was added at 80 μL/well and the plate will be incubated at room temperature for 15 min. The plate was rinsed three times in tap water to stop the developing reaction. After the final wash, Red Developer Solution (Cell Technology Limited, OH, USA) was added at 80 μL/well and the plate was incubated at room temperature for 5-10 min. The plate was rinsed three times to stop the developing reaction. The plate was air-dried for 24 hours face down on paper towels on the bench top. The spots on the plate representing splenocytes expressing IFNγ (red) or IL-5 (blue) were quantified using the CTL-Immunospot plate reader (ImmunoSpot 7.0.23.2 Analyzer Professional DC\ImmunoSpot 7, Cellular Technology Limited) and software (CTL Switchboard 2.7.2).

### Example 5—Phase I Clinical Trial

In this example, a vaccine to SARS-CoV-2, the novel coronavirus causing COVID-19 disease, is evaluated. The vaccine is administered as drops or a spray in the nose. Specifically, the study analyzes the safety of, and the immune response to, the vaccine when administered to healthy adults between the ages of 18 and 69 years. who are seronegative to SARS-CoV-2.

Cohort A (18-55 years) will enroll first. The first 10 participants (Group 1) will receive Dosage 1 of vaccine. After review of Group 1 safety data through Day 3, the next 20 participants (Group 2) receive Dosage 2 of vaccine. After review of Group 2 safety data through Day 3, the final group of 50 participants (Group 3) in Cohort A receive Dosage 3 of vaccine. A subgroup in Group 3 receive Dosage 3 of vaccine via nasal spray, whereas the remainder of participants receive administration by nasal drops. A 2nd subgroup in Cohort A receive a 2^{nd}, identical vaccine dose at Day 36, whereas the remainder of participants receive a single dose of vaccine (at Day 1).

After review of Cohort A safety data through Day 15, Cohort B (56-69 years) enroll. The first 10 participants (Group 4) receive Dosage 1 of vaccine. After review of Group 4 safety data through Day 3, the next 20 participants (Group 5) receive Dosage 2 of vaccine. After review of Group 5 safety data through Day 3, the final group of 20 participants (Group 6) in Cohort A receive Dosage 3 of vaccine. All participants in Cohort B receive a single dose of vaccine and are administered the dose by nasal drops. Within each group of Cohorts A and B, a sentinel dosing approach will be implemented as an additional safety measure.

TABLE 7

| Arm | Intervention |
|---|---|
| Experimental: Cohort A/Dosage Group 1 | Biological/Vaccine: vaccine against SARS- |
| (intranasal drops)/Single Dose | CoV-2 [MV-014-212] Dosage 1, Single Dose, |
| Participants in this arm (18-55 years) receive | Intranasal Drops |
| a single intranasal dose of the SARS-CoV-2 | Single intranasal dose on Day 1, by intranasal |
| vaccine at Dosage 1 in the form of intranasal | drops |
| drops on Day 1. | |
| Experimental: Cohort A/Dosage Group 2 | Biological/Vaccine: vaccine against SARS- |
| (intranasal drops)/Single Dose | CoV-2 [MV-014-212] Dosage 2, Single Dose, |
| Participants in this arm (18-55 years) receive | Intranasal Drops |
| a single intranasal dose of the SARS-CoV-2 | Single intranasal dose on Day 1, by intranasal |
| vaccine at Dosage 2 in the form of intranasal | drops |
| drops on Day 1. | |
| Experimental: Cohort A/Dosage Group 3a | Biological/Vaccine: vaccine against SARS- |
| (intranasal drops)/Single Dose | CoV-2 [MV-014-212] Dosage 3, Single Dose, |
| Participants in this arm (18-55 years) receive | Intranasal Drops |
| a single intranasal dose of the SARS-CoV-2 | Single intranasal dose on Day 1, by intranasal |
| vaccine at Dosage 3 in the form of intranasal | drops |
| drops on Day 1. | |
| Experimental: Cohort A/Dosage Group 3a | Biological/Vaccine: vaccine against SARS- |
| (intranasal drops)/Two Doses | CoV-2 [MV-014-212] Dosage 3, Two Doses, |
| Participants in this arm (18-55 years) receive | Intranasal Drops |
| an intranasal dose of the SARS-CoV-2 | Intranasal dose on Day 1 by intranasal drops. |
| vaccine at Dosage 3 in the form of intranasal | Followed by a second, identical dose on Day |
| drops on Day 1. These participants receive a | 36 by intranasal drops |
| second, identical dose of the SARS-CoV-2 | |
| vaccine at Dosage 3 in the form of intranasal | |
| drops on | |
| Day 36. | |
| Experimental: Cohort A/Dosage Group 3b | Biological/Vaccine: vaccine against SARS- |
| (intranasal spray)/Single Dose | CoV-2 [MV-014-212] Dosage 3, Single Dose, |
| Participants in this arm (18-55 years) receive | Intranasal Spray |
| a single intranasal dose of the SARS-CoV-2 | Single intranasal dose on Day 1, by intranasal |
| vaccine at Dosage 3 in the form of a nasal | spray |
| spray on Day 1. | |
| Experimental: Cohort B/Dosage Group 4 | Biological/Vaccine: vaccine against SARS- |
| (intranasal drops)/Single Dose | CoV-2 [MV-014-212] Dosage 1, Single Dose, |
| Participants in this arm (56-69 years) receive | Intranasal Drops |
| a single intranasal dose of the SARS-CoV-2 | Single intranasal dose on Day 1, by intranasal |
| vaccine at Dosage 1 in the form of intranasal | drops |
| drops on Day 1. | |
| Experimental: Cohort B/Dosage Group 5 | Biological/Vaccine: vaccine against SARS- |
| (intranasal drops)/Single Dose | CoV-2 [MV-014-212] Dosage 2, Single Dose, |
| Participants in this arm (56-69 years) receive | Intranasal Drops |
| a single intranasal dose of the SARS-CoV-2 | Single intranasal dose on Day 1, by intranasal |
| vaccine at Dosage 2 in the form of intranasal | drops |
| drops on Day 1. | |
| Experimental: Cohort B/Dosage Group 6 | Biological/Vaccine: vaccine against SARS- |
| (intranasal drops)/Single Dose | CoV-2 [MV-014-212] Dosage 3, Single Dose, |
| Participants in this arm (56-69 years) receive | Intranasal Drops |
| a single intranasal dose of the SARS-CoV-2 | Single intranasal dose on Day 1, by intranasal |
| vaccine at Dosage 3 in the form of intranasal | drops |
| drops on Day 1. | |

### Outcome Measures

The Primary Outcome Measures that are determined include solicited adverse events (AEs), unsolicited AEs, serious adverse events (SAEs), medically attended adverse events (MAEs), and change in serum neutralizing antibody titers against the vaccine-encoded SARS-CoV-2 S protein. Solicited and unsolicited AEs are determined in the period immediately post-vaccination. SAEs and MAEs are determined throughout the full study duration (about 1 year). The change in serum titer of neutralizing antibodies against vaccine-encoded SARS-CoV-2 S protein are determined from baseline through day 29, an average of five (5) weeks.

The frequency of solicited AEs are measured, categorized by severity. Solicited AEs are predefined AEs that may occur after vaccine administration.

The frequency of unsolicited AEs are measured, categorized by severity. Unsolicited AEs are any untoward medical occurrences in a participant administered the vaccine, regardless of causal relationship to the vaccine. Unsolicited AEs can include unfavorable and unintended signs (including abnormal laboratory findings), symptoms, or diseases temporally associated with the use of the vaccine.

The frequency of SAEs are measured, categorized by vaccine-relatedness. SAEs are AEs, whether considered causally related to the vaccine or not, that threaten life or result in any of the following: death, inpatient hospitalization or prolongation of existing hospitalization, persistent or significant incapacity or substantial disruption of the ability to conduct normal life functions, or congenital anomaly/birth defect.

The frequency of MAEs are measured, categorized by vaccine-relatedness. MAEs are AEs, whether considered causally related to the vaccine or not, with unscheduled medically attended visits, such as urgent care visits, acute primary care visits, emergency department visits, or other previously unplanned visits to a medical provider. Scheduled medical visits such as routine physicals, wellness checks, ‘check-ups’, and vaccinations, are not considered MAEs.

Change in serum neutralizing antibody (nAb) titers against vaccine-encoded SARS-CoV-2 S protein are measured per participant from baseline through day 29, an average of five (5) weeks.

Secondary Outcome Measures that are determined include (1) change in serum binding antibody concentrations against vaccine-encoded SARS-CoV-2 S protein, (2) frequency, magnitude, and duration of potential vaccine virus shedding.

Change in serum binding antibody concentrations are measured per participant from baseline through day 29, an average of five (5) weeks.

The frequency of any post-vaccination shedding of vaccine virus (as detected by viral culture) are measured per dosage group and overall from baseline through Day 29, an average of four (4) weeks. If post-vaccination shedding of vaccine virus is detected by culture, peak viral titer (measured in plaque forming units, PFU) will be measured per dosage group and overall from baseline through Day 29, an average of four (4) weeks. If post-vaccination shedding of vaccine virus is detected by culture, duration of shedding (in days) are measured per dosage group and overall from baseline through Day 29, an average of four (4) weeks.

Eligibility Criteria for this Study
Ages Eligible for Study: 18 Years to 69 Years
Sexes Eligible for Study: All
Gender Based: No
Accepts Healthy Volunteers: Yes

Inclusion Criteria:
Healthy adults ≥18 and <56 years (Cohort A) and 56 years and <70 years (Cohort B) as determined at the day of signing informed consent
SARS-CoV-2 RT-PCR (nasal swab) negative at Day 1 pre-dose
Women of childbearing potential (WOCBP) or male subjects with partners who are WOCBP must agree to practice contraception during their study participation from the signing of informed consent for at least 3 months after the final MV-014-212 administration.
Written informed consent

Exclusion Criteria:
Diagnosis of chronic pulmonary disease (e.g. chronic obstructive pulmonary disease, asthma, pulmonary fibrosis, cystic fibrosis). Resolved childhood asthma is not exclusionary.
Immunocompromised state due to comorbidities or other conditions as detailed in the study protocol
Nasal obstruction (including due to anatomic/structural causes, acute or chronic rhinosinusitis, or other causes)
Healthcare worker, long-term care or nursing home facility resident or employee, member of an emergency response team, or other occupation with high risk of exposure to SARS-CoV-2, and those working outside the home in customer facing occupations (e.g. waiter, cashier or store clerk, public transportation or taxi driver)
Positive serum pregnancy test during Screening and/or positive urine pregnancy test on Day 1
Breastfeeding during any period of study participation
Occupational or household exposure to children <5 years of age or to immunocompromised persons
Any medical disease or condition that, in the opinion of the PI, precludes study participation. This includes acute, subacute, intermittent or chronic medical disease or condition that would place the subject at an unacceptable risk of injury, render the subject unable to meet the requirements of the protocol, or may interfere with the evaluation of responses or the subject's successful completion of this trial

It is expected that subjects inoculated with MV-014-212 will exhibit an increase in serum titer of neutralizing antibodies against vaccine-encoded SARS-CoV-2 S protein as well as an increase in serum binding antibody concentrations against vaccine-encoded SARS-CoV-2 S protein.

Sequences provided in the sequence listing herein are shown in TABLE 8:

TABLE 8

| SEQ ID | |
|---|---|
| NO: | Description |
| 1 | Spike protein from insert 210 |
| 2 | Spike protein from insert 211 |
| 3 | Spike protein from insert 212 |
| 4 | Spike protein from insert 220 |
| 5 | Spike protein from insert 230 |
| 6 | Spike protein from insert 240 |
| 7 | DNA encoding Spike protein from insert 210 |
| 8 | DNA encoding Spike protein from insert 211 |
| 9 | DNA encoding Spike protein from insert 212 |
| 10 | DNA encoding Spike protein from insert 220 |
| 11 | DNA encoding Spike protein from insert 230 |
| 12 | DNA encoding Spike protein from insert 240 |
| .3 | Vaccine candidate MV-014-210 antigenome (DNA) |
| 14 | Vaccine candidate MV-014-211 antigenome (DNA) |
| 15 | Vaccine candidate MV-014-212 antigenome (DNA) |
| 16 | Vaccine candidate MV-014-220 antigenome (DNA) |
| 17 | Vaccine candidate MV-014-230 antigenome (DNA) |
| 18 | Vaccine candidate MV-014-240 antigenome (DNA) |
| 19 | GGGGGG |
| 20 | GGGGT |
| 21 | GGGPPP |
| 22 | GGGAPPP |
| 23 | Wild type coronavirus spike protein |
| 24 | DNA encoding wild type spike protein |
| 25 | KARSTPVTLSKDQLSGINNIAFSN-RSV F protein cytoplasmic tail (subgroup A) |
| 26 | KARSTPITLSKDQLSGINNIAFSN-RSV F protein cytoplasmic tail (subgroup B) |
| 27 | IMITTIIIVIIVILLSLIAVGLLLYC-RSV F protein TM domain (subgroup A) |
| 28 | IMITAIIIVIIVVLLSLIAIGLLLYC-RSV F protein TM domain (subgroup B) |
| 29 | GKSTTN |
| 30 | GKSTTNIMITTIIIVIIVILLSLIAVGLLLYCKARSTPVTLSKDQLSGINNIAFSN |
| | (subgroup A) |
| 31 | GKSTTNIMITAIIIVIIVVLLSLIAIGLLLYCKARSTPITLSKDQLSGINNIAFSN |
| | (subgroup B) |
| 32 | GLLLYCKARSTPVTLSKDQLSGINNIAFSN (subgroup A) |
| 33 | YCKARSTPVTLSKDQLSGINNIAFSN (subgroup A) |
| 34 | CKARSTPVTLSKDQLSGINNIAFSN (subgroup A) |
| 35 | KARSTPVTLSKDQLSGINNIAFSN (subgroup A) |
| 36 | ARSTPVTLSKDQLSGINNIAFSN (subgroup A) |
| 37 | GLLLYCKARSTPITLSKDQLSGINNIAFSN (subgroup B) |
| 38 | YCKARSTPITLSKDQLSGINNIAFSN (subgroup B) |
| 39 | CKARSTPITLSKDQLSGINNIAFSN (subgroup B) |
| 40 | KARSTPITLSKDQLSGINNIAFSN (subgroup B) |
| 41 | ARSTPITLSKDQLSGINNIAFSN (subgroup B) |
| 42 | RQSR |
| 43 | RRRR |
| 44 | codon-deoptimized NS1 gene |
| 45 | codon-deoptimized NS2 gene |
| 46 | BAC DB1 Quad mKate |
| 47 | Insert 210 |
| 48 | Insert 211 |
| 49 | Insert 212 |
| 50 | Insert 220 |
| 51 | Insert 230 |
| 52 | Insert 240 |
| 53 | Insert 300 |
| 54 | Full vector encoding RSV plus insert 210 (BAC DB1 Quad mKate background) |
| 55 | Full vector encoding RSV plus insert 211 (BAC DB1 Quad mKate background) |
| 56 | Full vector encoding RSV plus insert 212 (BAC DB1 Quad mKate background) |
| 57 | Full vector encoding RSV plus insert 220 (BAC DB1 Quad mKate background) |
| 58 | Full vector encoding RSV plus insert 230 (BAC DB1 Quad mKate background) |
| 59 | Full vector encoding RSV plus insert 240 (BAC DB1 Quad mKate background) |
| 50 | Wild type 300 insert (mKate) in BAC backbone |
| 61 | Wild type 300 insert in RSV backbone (MV-014-300 antigenome DNA, Kateless). |
| 62 | Spike protein MV-014-212-B.1.351 |
| 63 | Spike nucleotide MV-014-212-B.1.351 |
| 64 | MVK-014-212-B.1.351 |
| 65 | MV-014-212-B.1.351 |
| 66 | BAC MVK-014-212-B.1.351 |
| 67 | BAC MV-014-212-B.1.351 |
| 68 | Spike protein MV-014-212-B.1.1.7 |
| 69 | Spike nucleotide MV-014-212-B.1.1.7 |
| 70 | MVK-014-212-B.1.1.7 |
| 71 | MV-014-212-B.1.1.7 |
| 72 | BAC MVK-014-212-B.1.1.7 |
| 73 | BAC MV-014-212-B.1.1.7 |
| 74 | Spike protein MV-014-212-CAL20.C |
| 75 | Spike nucleotide MV-014-212-CAL20.C |
| 76 | MVK-014-212-CAL20.C |
| 77 | MV-014-212-CAL20.C |
| 78 | BAC MVK-014-212-CAL20.C |
| 79 | BAC MV-014-212-CAL20.C |
| 80 | Spike protein MV-014-212-P.1 |
| 81 | Spike nucleotide MV-014-212-P.1 |
| 82 | MVK-014-212-P.1 |
| 83 | MV-014-212-P.1 |
| 84 | BAC MVK-014-212-P.1 |
| 85 | BAC MV-014-212-P.1 |
| 86 | Spike protein MV-014-212-Del-Fur |
| 87 | Spike nucleotide MV-014-212-Del-Fur |
| 88 | MVK-014-212-Del-Fur |
| 89 | MV-014-212-Del-Fur |
| 90 | BAC MVK-014-212-Del-Fur |
| 91 | BAC MV-014-212-Del-Fur |
| 92 | Spike protein MV-014-212 R682Q |
| 93 | Spike nucleotide MV-014-212 R682Q |
| 94 | MVK-014-212 R682Q |
| 95 | MV-014-212 R682Q |
| 96 | BAC MVK-014-212 R682Q |
| 97 | BAC MV-014-212 R682Q |
| 98 | Spike protein MV-014-213 |
| 99 | Spike nucleotide MV-014-213 |
| 100 | MVK-014-213 |
| 101 | MV-014-213 |
| 102 | BAC MVK-014-213 |
| 103 | BAC MV-014-213 |
| 104 | MVK-014-210 |
| 105 | MVK-014-211 |
| 106 | MVK-014-212 |
| 107 | MVK-014-220 |
| 108 | MVK-014-230 |
| 109 | MVK-014-240 |
| 110 | Spike protein MV-014-215 |
| 111 | Spike nucleotide MV-014-215 |
| 112 | MVK-014-215 |
| 113 | MV-014-215 |
| 114 | BAC MVK-014-215 |
| 115 | BAC MV-014-215 |
| 116 | Influenza virus HA CT: X₁GX₂X₃X₄CX₅ICI; where X₁ is N or K; X₂ is S or N; X₃ |
| | is L, T, M, or C; X4 is Q or R; X5 is R, n or T |
| 117 | Influenza virus HA CT: NGSX₁X₂CX₃ICI; where X₁ is L, C or M. X₂ is Q or R; |
| | X₃ is R or N |
| 118 | Influenza virus HA CT: X₁GNX₂RCX₃ICI; where X₁ is K, N or R, X₂ is I or M, |
| | X₃ is N, T or Q |
| 119 | Parainfluenza virus F and/or HN protein CT: KLLTIVVANRNRMENFVYHK |
| 120 | Parainfluenza virus F and/or HN protein CT: MVAEDAPVRATCRVLFRTT |
| 121 | Measles virus F and/or HN protein CT: |
| | CCRGRCNKKGEQVGMSRPGLKPDLTGTSKSYVRSL |
| 122 | Measles virus F and/or HN protein CT: |
| | MSPQRDRINAFYKDNPHPKGSRIVINREHLMIDR |
| 123 | Mumps virus F and/or HN protein CT: YVATKEIRRINFKTNHINTISSSVDDLIRY |
| 124 | Mumps virus F and/or HN protein CT: |
| | MEPSKLFIMSDNATVAPGPVVNAAGKKTFRTCFR |
| 125 | Vesicular stomatitis virus (VSV) G protein CT: |
| | RVGIHLCIKLKHTKKRQIYTDIEMNRLGK |
| 126 | Rabies virus G protein CT: |
| | MTAGAMIGLVLIFSLMTWCRRANRPESKQRSFGGTGRNVSVTS |
| 127 | SG-F: CGATCTTGTAGATCTGTTCCTCAAACGAAC |
| 128 | SG-R: ATATTGCAGCAGTACGCACACACA |
| 129 | FAM-ACACTAGCCATCCTTACTGCGCTTCG-BHQ |
| 130 | RSV F Protein-C terminal domain comprising TM and CT in FIG. 2 |
| 131 | BAC MV-014-210 |
| 132 | BAC MV-014-211 |
| 133 | BAC MV-014-212 |
| 134 | BAC MV-014-220 |
| 135 | BAC MV-014-230 |
| 136 | BAC MV-014-240 |
| 137 | Furin cleavage site PRRA |
| 138 | Furin cleavage site mutation PQRA |
| 139 | FIG. 1 sequence |

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A chimeric protein comprising an ectodomain of a SARS-CoV-2 spike protein and a cytoplasmic tail portion of an RSV fusion (F) protein.

2. The chimeric protein of claim 1, wherein the chimeric protein comprises, in an N- to C-terminal direction, the ectodomain of the SARS-CoV-2 spike protein and the cytoplasmic tail portion of the RSV fusion (F) protein.

3. The chimeric protein of claim 1, wherein the chimeric protein further comprises a transmembrane portion of the SARS-CoV-2 spike protein.

4. The chimeric protein of claim 1, wherein the chimeric protein comprises a sequence selected from the group consisting of SEQ ID NOs: 1-6, 62, 68, 74, 80, 86, 92, 98, and 110 or a variant thereof having at least about 85% (e.g., at least about 90%, at least about 95%, at least about 96%, at least about 97%, at least about 98%, or at least about 99%) sequence identity to a sequence selected from the group consisting of SEQ ID NOs: 1-6, 62, 68, 74, 80, 86, 92, 98, and 110.

5. An immunogenic composition comprising live chimeric virus comprising a nucleic acid encoding the chimeric protein of any one of claims 1-4.

6. The immunogenic composition of claim 5, wherein the nucleic acid comprises a sequence selected from the group consisting of SEQ ID NOs: 7-12, 63, 69, 75, 81, 87, 93, 99, and 111 or a variant thereof having at least about 85% (e.g., at least about 90%, at least about 90%, at least about 95%, at least about 96%, at least about 97%, at least about 98%, or at least about 99%) sequence identity to a sequence selected from the group consisting of SEQ ID NOs: 7-12, 63, 69, 75, 81, 87, 93, 99, and 111, or an RNA counterpart of any of the foregoing, or a complementary sequence of any of the foregoing.

7. The immunogenic composition of claim 5 or claim 6, further comprising an NS1 and/or an NS2 protein of RSV.

8. The immunogenic composition of any one of claims 5-7, wherein the live chimeric virus does not comprise a gene that encodes RSV G protein.

9. The immunogenic composition of any one of claims 5-8, further comprising an adjuvant and/or other pharmaceutically acceptable carrier.

10. The immunogenic composition of claim 9, wherein the adjuvant is an aluminum gel, aluminum salt, or monophosphoryl lipid A.

11. The immunogenic composition of claim 9, wherein the adjuvant is an oil-in-water emulsion optionally comprising α-tocopherol, squalene, and/or a surfactant.

12. A method for immunizing a subject against a SARS-CoV-2 virus, the method comprising administering to the subject an effective amount of an immunogenic composition of any one of claims 4-11.

13. The method of claim 12, wherein the administration is intranasal administration.

14. The method of claim 12 or 13, wherein the immunogenic composition is administered a dose of between about 10³ and about 10⁶.

15. The method of any one of claims 12-14, wherein administration of the immunogenic composition induces a SARS-CoV-2 spike-specific mucosal IgA response or generates serum neutralizing antibodies.

16. A nucleic acid encoding the chimeric protein of claim 1 or claim 2.

17. The nucleic acid of claim 16 comprising a sequence selected from the group consisting of SEQ ID NOs: 7-12, 63, 69, 75, 81, 87, 93, 99, and 111 or a variant thereof having at least about 85% (e.g., at least about 90%, at least about 95%, at least about 96%, at least about 97%, at least about 98%, or at least about 99%) sequence identity to a sequence selected from the group consisting of SEQ ID NOs: 7-12, 63, 69, 75, 81, 87, 93, 99, and 111, or an RNA counterpart of any of the foregoing, or a complementary sequence of any of the foregoing.

18. A vector comprising a nucleic acid of claim 16 or claim 17.

19. The vector of claim 18 selected from a plasmid or a bacterial artificial chromosome.

20. The vector of claim 19, wherein the vector is a bacterial artificial chromosome comprising a sequence selected from the group consisting of SEQ ID NOs: 54-59, 66, 67, 72, 73, 78, 79, 84, 85, 90, 91, 96, 97, 102, 103, 114, 115, and 131-136 or a variant thereof having at least about 85% (e.g., at least about 90%, at least about 95%, at least about 96%, at least about 97%, at least about 98%, or at least about 99%) sequence identity to a sequence selected from the group consisting of SEQ ID NOs: 54-59, 66, 67, 72, 73, 78, 79, 84, 85, 90, 91, 96, 97, 102, 103, 114, 115, and 131-136.

21. An isolated recombinant particle comprising an NS1 and/or an NS2 protein of RSV and the chimeric F protein of claim 1 or claim 2.

22. The isolated recombinant particle of claim 21, comprising a live attenuated chimeric RSV-SARS-CoV-2 genome or antigenome.

23. A live attenuated chimeric RSV-SARS-CoV-2 antigenome comprising a sequence selected from the group consisting of SEQ ID NOs: 13-18, 65, 71, 77, 83, 89, 95, 101, 104-109, and 113 or a variant thereof having at least about 85% (e.g., at least about 90%, at least about 95%, at least about 96%, at least about 97%, at least about 98%, or at least about 99%) sequence identity to a sequence selected from the group consisting of SEQ ID NOs: 13-18, 65, 71, 77, 83, 89, 95, 101, 104-109, and 113, or an RNA counterpart of any of the foregoing, or a complementary sequence of any of the foregoing.
